(19) United States
(12) Patent Application Publication
(10) Pub. No.: US 2016/0183981 A1
(43) Pub. Date: Jun. 30, 2016

(54) METHODS AND DEVICES FOR ANCHORING SPINAL RODS

(71) Applicant: DePuy Synthes Products, Inc., Raynham, MA (US)

(72) Inventors: Fridolin Schlaepfer, Oberdorf (CH); Markus Hunziker, Oberdorf (CH)

(21) Appl. No.: 14/975,428

(22) Filed: Dec. 18, 2015

Related U.S. Application Data
(63) Continuation of application No. 62/097,217, filed on Dec. 29, 2014.

(51) Int. Cl. A61B 17/70

(52) U.S. Cl. CPC A61B 17/707; A61B 17/7056

(57) ABSTRACT

Methods and devices are provided for anchoring spinal rods. In general, devices are provided that can be configured to attach to a rib of a patient. In some embodiments, a device configured to attach to a rib of a patient to facilitate anchoring of a spinal rod coupled thereto can include first and second connectors each configured to attach to a rib of a patient. The first and second connectors can be independent elements and can be configured to be movable relative to one another. A spinal rod can be configured to couple to the device, to move relative to the first and second connectors and the rib after the first and second connectors have been connected to the rib, and to attach to a second device attached to another portion of the patient's anatomy, thereby securing the spinal rod in a fixed position to facilitate treatment.

### FIELD

The present invention relates to methods and devices for anchoring spinal rods.

### BACKGROUND

Expandable prosthetic rib (EPR) devices are designed to mechanically stabilize the thorax of a patient to correct three-dimensional thoracic deformities, and to provide improvements in volume for respirations and lung growth in infantile and juvenile patients diagnosed with thoracic insufficiency syndrome. Once the initial implantation procedure is complete and an EPR device is implanted into a patient's body, the EPR device may allow for some adjustability, such as expansion, anatomic distraction, and replacement of some components of the EPR device through subsequent surgical procedures, which are generally less invasive than the initial implantation procedure. Available EPR devices are typically elongated devices that are attached to one or more of the patient's ribs and to the patient's pelvis, such that the EPR device extends along the spine of the patient.

EPR devices are typically attached to the patient's rib via a cradle, which extends from the posterior side towards the anterior side of the patient. The cradle typically substantially encloses the patient's rib to prevent the EPR device from becoming displaced during breathing or other movements of the patient. Ribs in infants and children are typically very small, so the EPR device that attaches to such a very small rib can damage the rib and/or adjacent bodily structures due to high load distribution, can damage the rib and/or adjacent bone due to the EPR device having a size that is large enough to catch on the very small rib and/or adjacent bone, and/or can puncture the patient's pleura due to the EPR device having a size that is large enough to move into dangerous contact with the pleura during the patient's normal activities post-implantation and thereby cause the patient's lung to collapse due to the punctured pleura.

Some available EPR devices have an end that is typically attached to the patient's pelvis via a S-shaped hook, which S-shaped hook may generally extend from the posterior side of the patient to the anterior side of the patient. Available S-shaped hooks typically rest on top of the iliac crest of the patient, and due to their S-shape, tend to inherently have a limited contact area with the iliac crest.

Accordingly, there is a need for improved methods and devices for anchoring spinal rods.

### SUMMARY

The present invention generally provides methods and devices for anchoring spinal rods. In one embodiment, a surgical device is provided that includes a first connector having a first seat configured to seat a bone, and a second connector having a second seat and a third seat each configured to seat the bone. The second connector can be configured to move relative to the first connector when the first seat is seating the bone so as to cause the second and third seats to seat the bone with the first seat being positioned between the second and third seats such that the bone connector is configured to have three points of contact with the bone when the first and second connectors are connected to the bone. One of the first and second connectors can be configured to seat an elongate rod such that the seated rod is movable relative to at least one of the first and second connectors in at least one direction.

The device can have any number of variations. For example, each of the first, second, and third seats can include an arcuate extension. For another example, the first seat can include an elongate post and each of the second and third seats can include an arcuate extension. For yet another example, the first and second connectors can be configured to be secured in a fixed position relative to one another so as to secure the bone seated in the first, second, and third seats. For still another example, the movement of the second connector relative to the first connector can include rotational movement of the second connector about a pivot point defined by the first connector. For another example, the movement of the second connector relative to the first connector can include longitudinal sliding movement of the second connector along a longitudinal axis defined by the first connector. For yet another example, the first and second connectors can be movable along a longitudinal length of the seated rod. For another example, the seated rod can be angularly movable relative to the first and second connectors. For yet another example, the first connector can be configured to seat the elongate rod. For still another example, the second connector can be configured to seat the elongate rod.

In some embodiments, the device can include a second bone connector that includes a third connector having a fourth seat configured to seat a second bone, and a fourth connector having a fifth seat and a sixth seat each configured to seat the second bone. The fourth connector can be configured to move relative to the third connector when the fourth seat is seating the second bone so as to cause the fifth and sixth seats to seat the second bone with the fourth seat being positioned between the fifth and sixth seats such that the second bone connector is configured to have three points of contact with the second bone when the third and fourth connectors are connected to the second bone. One of the third and fourth connectors can be configured to seat the elongate rod.

In another embodiment, a surgical device is provided that includes a first connector including a first seat configured to seat a rib, and a second connector including a second seat configured to seat the rib. The first and second connectors can be configured to be secured in a fixed position relative to one another with the rib seated by the first and second seats. The device can also include an elongate rod configured to be seated by one of the first and second connectors and be movable relative thereto when the first and second connectors are in the fixed position relative to one another so as to allow adjustability of the elongate rod relative to the rib.

The device can vary in any number of ways. For example, the movement of the elongate rod relative to the first and second connectors can include sliding movement of the elongate rod along a longitudinal axis of the elongate rod. For another example, the movement of the elongate rod relative to the first and second connectors can include pivoting movement of the elongate rod about a pivot point defined by a connection point between one end of the elongate rod and the one of the first and second connectors that seats the elongate rod. For yet another example, the one of the first and second connectors can be configured to have the elongate rod side loaded thereto. For still another example, the one of the first and second connectors can be configured to have the elongate rod end loaded thereto. For another example, the first and second seats can each include a curved seat. For yet another example, one of the first and second seats can include a curved seat and the other of the first and second seats can include an elongate post.

In some embodiments, the first connector can include a first opening therein, and the second connector can include a second opening therein. The device can also include a set screw configured to be received in the first and second openings so as to fix the first and second connectors in the fixed position relative to one another with the rib seated by the first and second seats.

In some embodiments, the device can include a third connector including a third curved seat configured to receive a second rib therein, and a fourth connector including a fourth curved seat configured to receive the second rib therein. The elongate rod can be configured to be seated by one of the third and fourth connectors and be movable relative thereto so as to allow adjustability of the elongate rod relative to the second rib.

In another embodiment, a surgical device is provided that includes a first connector including a first curved seat configured to receive a bone therein, and a second connector that is discrete from the first connector such that each of the first and second connectors are configured to be movable independent of each other. The second connector can include a second curved seat configured to receive the bone therein. The second connector can be configured to rotate relative to the first connector, having the bone received in the first curved seat thereof, about a pivot point defined by the first connector so as to cause the second curved seat to rotate relative to the first curved seat so as to receive the bone therein.

The device can vary in any number of ways. For example, the device can include an elongate rod configured to be seated by one of the first and second connectors and configured to be adjusted in position relative to the first and second connectors having the bone received in the first and second curved seats. For another example, the second connector can include a third curved seat configured to receive the bone therein. For yet another example, the second connector can be configured to slide linearly relative to the first connector after the rotation of the second connector.

In another embodiment, a surgical device is provided that includes a first connector including a first bone securing member, and a second connector including a second bone securing member. The second connector can be configured to slide linearly with respect to the first connector so as to adjust a spacing between the second bone and the first bone securing member and thereby engage a bone between the first and second bone securing members. The first and second connectors can be configured to be secured in a fixed position relative to one another so as to fix the bone in position between the first and second bone securing members. One of the first and second bone securing members can include a curved member extending from the one of the first and second connectors, and the other of the first and second bone securing members can include an elongate post.

The device can have any number of variations. For example, the device can include an elongate rod configured to be seated by one of the first and second connectors and configured to be adjusted in position relative to the first and second connectors having the bone positioned between the first and second bone securing members. For another example, the one of the first and second bone securing members can include a second curved member extending from the one of the first and second connectors.

In another embodiment, a surgical device is provided that includes a first component having a first interface configured to grasp one side of an anatomical structure, and a second component having a second interface configured to grasp an opposite side of the anatomical structure. The first and second components can be separate entities which can be mechanically connected by a mechanical connection having at least one degree of movement. At least one of the first and second components can include a third interface configured to connect to an object at a second mechanical connection. The second mechanical connection can provide at least one degree of movement.

The device can vary in any number of ways. For example, the at least one degree of movement can be at least partially locked. For another example, the at least one degree of movement can be at least partially mobile. For yet another example, the at least one degree of movement can be at least partially mobile. For still another example, the mechanical connection can provide the at least one degree of movement only as translation. For another example, the mechanical connection can include a hinge joint. For yet another example, the mechanical connection can include a ball-and-socket joint. For another example, the mechanical connection can not allow any movement between the first and second components. For yet another example, the first interface can be geometrically complementary to the anatomical structure where the first interface grasped the anatomical structure. For another example, the second interface can be geometrically complementary to the anatomical structure where the second interface grasped the anatomical structure. For yet another example, the first interface can be malleable in the form of a foam or in the form of a gel-like substance. For another example, the second interface can be malleable in the form of a foam or in the form of a gel-like substance. For still another example, a pressure distribution at the first and second interfaces can be uniform. For yet another example, the first and second interfaces can be convex. For another example, the first interface can include a double blade. For still another example, stress provoked in the anatomical structure by the first and second components can be less than a mechanical threshold of the anatomical structure. For yet another example, stress provoked in the anatomical structure by the first and second components can be less than a threshold for provoking a negative biological reaction in a subject including the anatomical structure. For another example, the object can include a longitudinal carrier. For yet another example, the at least one degree of movement can be configured to be at least partially locked. For another example, wherein the at least one degree of movement can be configured to be at least partially mobile. For still another example, the at least one degree of movement of the second mechanical connection can be configured to allow only translation. For yet another example, the second mechanical connection can include a hinge joint. For another example, the second mechanical connection can include a ball-and-socket joint. For still another example, the second mechanical connection can be configured to prevent any relative movement between the at least one of the first and second components and the object.

For another example, the device can include a third component that is a separate entity from the first and second components. The second mechanical connection can be via the third component. For yet another example, the third component can include a rod-receiving channel. The rod-receiving channel can be open vertical to a longitudinal axis of the third connector, or the rod-receiving channel can be open along the longitudinal axis of the third connector. For another example, the third component can share a common interface with the at least one of the first and second components. In some embodiments, the common interface can includes a hinge joint. In some embodiments, the common interface can includes a ball-and-socket joint. In some embodiments, the common interface can be configured to prevent movement of the third component relative to the at least one of the first and second components. In some embodiments, the common interface can be configured to allow the at least one of the first and second components to click onto the third component to form the second mechanical connection.

In another embodiment, a surgical device is provided that includes a first section including a first surface, and a second section including a second surface that is diametrically opposed to the first surface such that the first and second surfaces are configured to engulf an anatomical structure from two opposed sides of the anatomical structure and by fitting around a complementary part of the anatomical structure. At least one of the first and second sections can include an interface configured to connect to an object at a mechanical connection. The mechanical connection can provide at least one degree of movement.

The device can have any number of variations. For example, the first surface can be geometrically complementary to the complementary part of the anatomical structure. For another example, the second surface can be geometrically complementary to the complementary part of the anatomical structure. For yet another example, the first surface can be malleable and can be in the form of a foam or in the form of a gel-like substance. For still another example, the second surface can be malleable and can be in the form of a foam or in the form of a gel-like substance. For another example, a pressure distribution at the first and second interfaces can be uniform. For still another example, the first and second surfaces can be convex. For yet another example, the first surface can include a double blade. For still another example, stress provoked in the anatomical structure by the first and second sections can be less than a mechanical threshold of the anatomical structure. For another example, stress provoked in the anatomical structure by the first and second sections can be less than a threshold for provoking a negative biological reaction in a subject including the anatomical structure. For yet another example, the object can include a longitudinal carrier. For another example, the at least one degree of movement can be configured to be at least partially locked. For yet another example, the at least one degree of movement can be configured to be at least partially mobile. For still another example, the at least one degree of movement can be configured to allow only translation. For another example, the mechanical connection can include a hinge joint. For still another example, the mechanical connection can include a ball-and-socket joint. For yet another example, the mechanical connection can be configured to prevent any relative movement between the at least one of the first and second sections and the object.

For another example, the device can include a connector that is a separate entity from the first and second sections. The mechanical connection can be provided by the connector. In some embodiments, the connector can include a rod-receiving channel. The rod-receiving channel can be open vertical to a longitudinal axis of the connector, or the rod-receiving channel can be open along the longitudinal axis of the connector. In some embodiments, the connector can share a common interface with the at least one of the first and second sections. In some embodiments, the common interface can include a hinge joint. In some embodiments, the common interface can include a ball-and-socket joint. In some embodiments, the common interface can be configured to prevent movement of the connector relative to the at least one of the first and second sections. In some embodiments, the common interface can be configured to allow the at least one of the first and second sections to click onto the connector to form the mechanical connection.

In another aspect, a surgical system is provided that in one embodiment includes a first bone connector configured to connect to a first bone. The first bone connector can include a first connector having a first curved seat configured to seat the first bone, and a second connector configured to attach to the first connector. The second connector can have a second curved seat and a third curved seat each configured to seat the first bone. The second and third seats can be laterally offset from one another and can each be laterally offset from the first curved seat when the first and second curved seats seat the bone. The device can also include a second bone connector configured to connect to a second bone, and an elongate rod configured to be seated by the first bone connector and by the second bone connector so as to extend between the first and second bones.

The system can have any number of variations. For example, the elongate rod can be one of angularly adjustable and longitudinally slidable relative to the first bone connector when seated by the first bone connector. The elongate rod can be one of angularly adjustable and longitudinally slidable relative to the second bone connector when seated by the second bone connector. For another example, the first bone can be one of a first rib, a first pelvic bone, and a first vertebra, and the second bone can be one of a second rib, a second pelvic bone, and a second vertebra. The first bone can be the first rib, and the second bone can be the second rib.

In another aspect, a surgical method is provided that in one embodiment includes engaging a rib with a first seat of a first connector and subsequently rotating a second connector relative to the first connector so as to move a second seat of a second connector toward the first seat and engage the rib with the second seat. The method can also include, with the rib engaged by the first and second seats, securing the first and second connectors in a fixed position relative to one another. The method can also include adjusting a position of an elongate rod coupled to the first and second connectors relative to the first and second connectors.

The method can vary in any number of ways. For example, the elongate rod can be adjusted after the securing. For another example, the elongate rod can be adjusted before the securing. For yet another example, the adjusting can include pivoting the elongate rod about an end of the elongate rod. For still another example, the adjusting can include longitudinally sliding the elongate rod.

### BRIEF DESCRIPTION OF THE DRAWINGS

The invention will be more fully understood from the following detailed description taken in conjunction with the accompanying drawings, in which:

FIG. 1 is a perspective view of one embodiment of a surgical device configured to attach to a bone and including first and second connectors, the second connector having an elongate rod coupled thereto;

FIG. 2 is another perspective view of the device of FIG. 1;

FIG. 3 is a perspective view of the device of FIG. 1 with the second connector rotated relative to the first connector;

FIG. 4 is a perspective view of the device of FIG. 3 with the second connector rotated closer to the first connector;

FIG. 5 is a perspective, partially transparent view of another embodiment of a surgical device configured to attach to a bone and including first and second connectors, the second connector having an elongate rod coupled thereto;

FIG. 6 is a perspective, partially transparent view of yet another embodiment of a surgical device configured to attach to a bone and including first and second connectors, the second connector having an elongate rod coupled thereto;

FIG. 7 is a perspective, partially transparent view of still another embodiment of a surgical device configured to attach to a bone and including first and second connectors, the second connector having an elongate rod coupled thereto, and the second connector being rotated relative to the first connector;

FIG. 8 is a perspective view of the device of FIG. 7 with the second connector rotated closer to the first connector;

FIG. 9 is a perspective view of the device of FIG. 8 with the second connector rotated closer to the first connector;

FIG. 10 is another perspective view of the device of FIG. 9;

FIG. 11 is a perspective view of the device of FIG. 10 with the second connector slid longitudinally away from to the first connector;

FIG. 12 is a perspective view of another embodiment of a surgical device configured to attach to a bone and including first and second connectors, the first connector having an elongate rod coupled thereto;

FIG. 13 is a perspective view of yet another embodiment of a surgical device configured to attach to a bone and including first and second connectors, the first connector having an elongate rod coupled thereto;

FIG. 14 is a perspective view of another embodiment of a surgical device configured to attach to a bone and including first and second connectors, the second connector having an elongate rod coupled thereto;

FIG. 15 is a perspective view of yet another embodiment of a surgical device configured to attach to a bone and including first and second connectors, the second connector having an elongate rod coupled thereto;

FIG. 16 is a perspective view of still another embodiment of a surgical device configured to attach to a bone and including first and second connectors, the second connector having an elongate rod coupled thereto;

FIG. 17 is a perspective view of another embodiment of a surgical device configured to attach to a bone and including first and second connectors, the second connector having an elongate rod coupled thereto;

FIG. 18 is a perspective, partially transparent view of the device of FIG. 17;

FIG. 19 is a perspective view of the device of FIG. 17 with the elongate rod moved to a different angular orientation relative to the second connector;

FIG. 20 is a perspective, partially transparent view of the device of FIG. 17 with the first connector slid longitudinally closer to the second connector;

FIG. 21 is a perspective, partially transparent view of the device of FIG. 20 with the first connector slid longitudinally closer to the second connector;

FIG. 22 is a schematic view of a partial human skeleton showing possible attachment locations of bone connectors to various bones of the skeleton;

FIG. 23 is a schematic view of a human vertebra showing possible attachment locations of bone connectors to various portions of the vertebra and to ribs extending therefrom;

FIG. 24 is a perspective view of one embodiment of a surgical device configured to attach to a bone and to facilitate cross connection to another bone, the device including first and second connectors, and the second connector having first and second elongate rods coupled thereto;

FIG. 25 is another perspective view of the device of FIG. 24;

FIG. 26 is a side schematic view of one embodiment of a receiver member;

FIG. 27 is a perspective view of the receiver member of FIG. 27;

FIG. 28 is a side schematic view of another embodiment of a receiver member;

FIG. 29 is a perspective view of the receiver member of FIG. 28;

FIG. 30 is a side schematic view of yet another embodiment of a receiver member;

FIG. 31 is a perspective view of the receiver member of FIG. 30;

FIG. 32 is a perspective view of one embodiment of a surgical device configured to attach to a bone and to facilitate cross connection to another bone, the device including first, second, and third connectors, and the device being coupled to a cross connection rod and the receiver member of FIG. 26, the receiver member being coupled to a spinal rod;

FIG. 33 is another perspective view of the device of FIG. 32;

FIG. 34 is a side view of the device of FIG. 32;

FIG. 35 is a perspective view of another embodiment of a surgical device configured to attach to a bone and to facilitate cross connection to another bone, the device including first, second, and third connectors, and the device being coupled to a cross connection rod and the receiver member of FIG. 26, the receiver member being coupled to a spinal rod;

FIG. 36 is a perspective view of the device of FIG. 35 with the second connector angularly adjusted in position relative to the first and third connectors;

FIG. 37 is a perspective view of the device of FIG. 36 with the second connector angularly adjusted in position relative to the first and third connectors;

FIG. 38 is a perspective view of the device of FIG. 36 with the receiver member adjusted in position relative to the first, second, and third connectors;

FIG. 39 is a perspective view of the device of FIG. 38 with the receiver member adjusted in position relative to the first, second, and third connectors;

FIG. 40 is a perspective view of first, second, third, and fourth surgical devices coupled together in a row from right to left, the first device being the device of FIG. 35 with the second connector angularly adjusted in position relative to the first and third connectors, the second device being another device of FIG. 35 with the second connector angularly adjusted in position relative to the first and third connectors and with the receiver member angularly adjusted in position relative to the first, second, and third connectors, the third device being another embodiment of a surgical device configured to attach to a bone and to facilitate cross connection to another bone, and the fourth device being a surgical device of FIG. 43 with a second connector of the device being angularly adjusted in position relative to the first and third connectors;

FIG. 41 is a top view of the first, second, third, and fourth surgical devices of FIG. 40;

FIG. 42 is a side view of the first, second, third, and fourth surgical devices of FIG. 40;

FIG. 43 is a perspective view of another embodiment of a surgical device configured to attach to a bone and to facilitate cross connection to another bone, the device including first, second, and third connectors, and the device being coupled to a cross connection rod and the receiver member of FIG. 30, the receiver member being coupled to a spinal rod;

FIG. 44 is a perspective view of the device of FIG. 43 with the second connector angularly adjusted in position relative to the first and third connectors;

FIG. 45 is a perspective view of the device of FIG. 44 with the receiver member rotated in position relative to the first, second, and third connectors;

FIG. 45A is a partially transparent view of the device and receiver member of FIG. 45;

FIG. 46 is a perspective view of another embodiment of a surgical device configured to attach to a bone and to facilitate cross connection to another bone, the device being coupled to a spinal rod and to a rod seating member;

FIG. 47 is a perspective view of the device of FIG. 46;

FIG. 48 is a perspective view of the rod seating member of FIG. 46;

FIG. 49 is a side, partially transparent view of the device of FIG. 46, the device seating a locking member therein locking the spinal rod and the rod seating member in a fixed position relative to the device;

FIG. 50 is another side, partially transparent view of the device of FIG. 46, the device seating a locking member therein locking the spinal rod and the rod seating member in a fixed position relative to the device;

FIG. 51 is a perspective view of the device of FIG. 46, the device seating a locking member therein locking the spinal rod and the rod seating member in a fixed position relative to the device;

FIG. 52 is a perspective view of the device of FIG. 51, the device seating a locking member therein locking the spinal rod and the rod seating member in a fixed position relative to the device;

FIG. 53 is a perspective view of the device of FIG. 52, the device seating a locking member therein locking the spinal rod and the rod seating member in a fixed position relative to the device;

FIG. 54 is a side view of the device of FIG. 51 and a side view of a traditional surgical device configured to attach to a bone and to facilitate cross connection to another bone, the traditional device being in a foreground in front of the device of FIG. 51;

FIG. 55 is a perspective view of another embodiment of a surgical device configured to attach to a bone and to facilitate cross connection to another bone, the device being coupled to a spinal rod, to another embodiment of a rod seating member, and to a locking member locking the spinal rod and the rod seating member in position relative to the device;

FIG. 56 is a perspective, partially transparent view of the device of FIG. 55;

FIG. 57 is a perspective view of the device of FIG. 55;

FIG. 58 is a perspective view of the rod seating member of FIG. 55;

FIG. 59 is a side view of the device of FIG. 55 with the rod seating member and the spinal rod adjusted in position relative to the device;

FIG. 60 is a side view of the device of FIG. 59 with the rod seating member and the spinal rod adjusted in position relative to the device;

FIG. 61 is a side view of the device of FIG. 60 with the rod seating member and the spinal rod adjusted in position relative to the device;

FIG. 62 is a side view of the device of FIG. 61 with the rod seating member and the spinal rod adjusted in position relative to the device;

FIG. 63 is a side view of the device of FIG. 55 coupled to a cross connection rod, and a side view of the traditional device of FIG. 54 coupled to the cross connection rod, a surgical tool being coupled to opposed cut-outs of the device of FIG. 55;

FIG. 64 is a top view of the device, traditional device, and tool of FIG. 63;

FIG. 65 is a perspective view of the device, traditional device, and tool of FIG. 63;

FIG. 66 is a perspective view of a distal portion of the tool of FIG. 63;

FIG. 67 is a perspective view of another embodiment of a surgical device configured to attach to a bone and to facilitate cross connection to another bone;

FIG. 68 is a side view of the device of FIG. 67 and the traditional device of FIG. 54, the device of FIG. 67 being coupled to the tool of FIG. 66 and to a cross connection rod, and the traditional device of FIG. 54 being coupled to the cross connection rod;

FIG. 69 is a side view of the device and the traditional device of FIG. 68, the tool being longitudinally adjusted in position relative to the device;

FIG. 70 is a side view of the device and the traditional device of FIG. 69, the tool being laterally and longitudinally adjusted in position relative to the device;

FIG. 71 is a side view of the device and the traditional device of FIG. 70, the tool being laterally and longitudinally adjusted in position relative to the device;

FIG. 72 is a side view of the device and the traditional device of FIG. 71, the tool being angularly adjusted in position relative to the device;

FIG. 73 is a side view of the device and the traditional device of FIG. 72, the tool being angularly adjusted in position relative to the device;

FIG. 74 is a side view of the device and the traditional device of FIG. 73, the tool being angularly adjusted in position relative to the device;

FIG. 75 is a side view of the device and the traditional device of FIG. 74, the tool being angularly, laterally, and longitudinally adjusted in position relative to the device;

FIG. 76 is a side view of the device and the traditional device of FIG. 75, the tool being laterally and longitudinally adjusted in position relative to the device;

FIG. 77 is a side view of the device and the traditional device of FIG. 76, the tool being laterally and longitudinally adjusted in position relative to the device;

FIG. 78 is a side view of the device and the traditional device of FIG. 75, the tool being angularly adjusted in position relative to the device;

FIG. 79 is a perspective view of the device of FIG. 55 coupled to another embodiment of a surgical tool;

FIG. 80 is a perspective, partially transparent view of the device and the tool of FIG. 79;

FIG. 81 is a perspective view of the device of FIG. 79 and of a distal portion of the tool of FIG. 79

FIG. 82 is a perspective, partially transparent view of the device and the distal portion of the tool of FIG. 81;

FIG. 83 is a perspective view of another embodiment of a surgical device attached to a vertebra and configured to facilitate cross connection to another bone, and a perspective view of the traditional device of FIG. 54 attached to the vertebra, the device being coupled to the receiver member of FIG. 26;

FIG. 84 is a side view of the device and the traditional device of FIG. 83, the receiver member being angularly adjusted in position relative to the device;

FIG. 85 is another perspective view of the device and the traditional device of FIG. 83;

FIG. 86 is a perspective view of another embodiment of a surgical device attached to a vertebra and configured to facilitate cross connection to another bone, and a perspective view of the traditional device of FIG. 54 attached to the vertebra, the device being coupled to a locking member;

FIG. 87 is a perspective view of another embodiment of a surgical device attached to a vertebra, and a perspective view of the traditional device of FIG. 54 attached to the vertebra;

FIG. 88 is a perspective view of the device and the traditional device of FIG. 87, the device being coupled to a spinal rod, to a first locking member locking the spinal rod in position relative to the device, and to a second locking member locking first and second connectors of the device in position relative to one another;

FIG. 89 is a perspective view of the traditional device of FIG. 87 and to a second traditional device attached to the vertebra;

FIG. 90 is a perspective view of two embodiments of surgical devices configured to anchor a spinal rod;

FIG. 91 is a side view of three embodiments of surgical devices configured to anchor a spinal rod;

FIG. 92 is a perspective view of the three devices of FIG. 91;

FIG. 93 is a perspective view of two embodiments of tandem surgical devices configured to anchor a spinal rod;

FIG. 94 is a side view of the two devices of FIG. 93;

FIG. 95 is a perspective view of another embodiment of a surgical device configured to anchor a spinal rod;

FIG. 96 is another perspective view of the device of FIG. 95;

FIG. 97 is yet another perspective view of the device of FIG. 95;

FIG. 98 is still another perspective view of the device of FIG. 95;

FIG. 99 is a perspective view of another embodiment of a surgical device configured to anchor a spinal rod;

FIG. 100 is another perspective view of the device of FIG. 99;

FIG. 101 is a perspective view of another embodiment of a surgical device configured to anchor a spinal rod;

FIG. 102 is a perspective view of the device of FIG. 101 with a first end of the device being angularly adjusted in position relative to a second, opposite end of the device;

FIG. 103 is a perspective view of the second end of the device of FIG. 102; and

FIG. 104 is a perspective view of the second end of the device of FIG. 103, a bone receiving area of the second end of the device being adjusted to be greater in size from FIG. 103.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various exemplary methods and devices are provided for anchoring spinal rods. In general, devices are provided that can be configured to attach to a rib of a patient to facilitate anchoring of an elongate rod, e.g., a spinal rod, coupled thereto. In some embodiments, a device configured to attach to a rib of a patient to facilitate anchoring of an elongate rod coupled thereto can include first and second connectors each configured to attach to a rib of a patient. The first and second connectors can be independent elements, which can facilitate secure engagement of the rib by the first and second connectors since each of the first and second connectors can be independently positioned relative to the rib. The first and second connectors can be configured to be movable relative to one another, which can help the first and second connectors each securely couple to the rib. A rod can be configured to couple to the device, thereby allowing the first and second connectors to secure the rod within the patient. The rod can be configured to move relative to the first and second connectors and the rib after the first and second connectors have been connected to the rib, thereby allowing the rod's position to be desirably adjusted in view of the patient's particular anatomy and the patient's particular treatment. The rod can be configured to attach to a second device attached to another portion of the patient's anatomy, e.g., another bone such as a rib, pelvic bone, or vertebra, thereby securing the rod in a fixed position to facilitate treatment, e.g., to facilitate correction of a thoracic deformity. The second device can be identical to the first device, or the second device can have another configuration. In embodiments in which the first and second devices are configured to attached to first and second ribs, respectively, the rod can be anchored without any device being attached to a bone of the patient, e.g., the pelvis or the vertebra, other than the first and second ribs in order to anchor the rod to, e.g., correct a spinal deformity of the patient. The first and second devices can thus cooperate to define a rib-to-rib connector configured to anchor the rod within the patient's body. In embodiments in which the first device is configured to attach to a rib and the second device is configured to attach to a pelvic bone, e.g., the ilium, the first and second devices can cooperate to define a rib-to-pelvis connector configured to anchor the rod within the patient's body. In embodiments in which the first device is configured to attach to a rib and the second device is configured to attach to a vertebra, the first and second devices can cooperate to define a rib-to-vertebra connector configured to anchor the rod within the patient's body.

In one embodiment of using the device, the first connector can be positioned to engage the rib, and with the rib engaged by the first connector, the second connector can be moved relative to the first connector so as to engage the rib between the first and second connectors. The rod coupled to the second connector can then be adjusted in position relative to the first and second connectors and the rib, which can help ensure that the rod is properly positioned for treatment.

In some embodiments, the device can be configured to have three points of contact with the rib engaged by the device's first and second connectors. The three points of contact can help distribute the load of the device on the rib to help prevent the rib from being damaged by the device. The device being configured to have three points of contact with a rib instead of one or two points of contact with the rib, the device can be configured to more evenly distribute the load on the rib, thereby reducing chances of damaging the rib. The three points of contact can be arranged axially along a longitudinal axis of the rib, which can facilitate distribution of the load along a longitudinal length of the rib rather than concentrating the load in a more localized axial position of the rib, such as with a device having two points of contact with a rib at a same axial position along the rib.

In some embodiments, at least one of the first and second connectors can include at least one arcuate extension configured to seat a rib so as to couple the rib thereto. In an exemplary embodiment, the first connector can include a seat in the form of one arcuate extension, and the second connector can include a seat in the form of two arcuate extensions. Each of the arcuate extensions can be configured to contact the rib such that the device has three points of contact with the rib. The three arcuate extensions can be configured to be arranged at different axial positions along the rib engaged thereby, which can help improve load distribution on the rib. In another exemplary embodiment, one of the first and second connectors can include a seat in the form of two arcuate extensions, and the other of the first and second connectors can include a seat in another form, e.g., an elongate post. The three seats can be configured to arranged at different axial positions along the rib engaged thereby, which can help improve load distribution on the rib.

In some embodiments, a device configured to attach to a bone of a patient to facilitate anchoring of an elongate rod coupled thereto can be configured to allow cross connection, such as cross connection to the patient's thoracic vertebra and go around the patient's transverse process. The device can be configured to couple to a first elongate rod, e.g., a spinal rod, configured to be coupled to a second device attached to a second part of the patient's anatomy (e.g., a bone other than the bone to which the first device is coupled), and can be configured to be coupled to a second elongate rod, e.g., a cross connector rod, configured to be coupled to a third device attached to a third part of the patient (e.g., a bone other than the bone to which the first device is coupled and other than the bone to which the second device is attached). The device can thus be versatile for use in a variety of different patient treatment plans. In some embodiments, a device configured for cross-connection can be configured to have three points of contact with a rib engaged by the device, e.g., by the device's first and second connectors.

In some embodiments, a device configured for cross-connection can include a first connector, a second connector configured to be movably coupled to the first connector, and a receiver element configured to be movably coupled to the first and second connectors and configured to receive a spinal rod. The receiver element can be configured to be monoaxially movable relative to the first and second connectors, or the receiver element can be configured to be polyaxially movable relative to the first and second connectors. The device can be configured to have a lower profile than traditional devices configured for cross-connection, which can help reduce chances of the device damaging the patient, e.g., damaging a bone, puncturing a pleura, etc., and/or can help make the device easier to implant since it can be more easily positioned in small spaces tam devices having larger profiles.

In some embodiments, a device configured for cross-connection can include a seating element configured to be movably coupled to the second connector and configured to movably seat the spinal rod therein. The seating element can be configured to facilitate positioning of the spinal rod relative to the second connector, which can help the spinal rod be desirably positioned relative to the patient's particular anatomy. The spinal rod can be configured to be fixed in position relative to the second connector by being secured in position between the receiver element and the seating element, such as by a set screw being screwed into the receiver element.

In some embodiments, a device configured for cross-connection can include an installation tool configured to facilitate insertion of and/or positioning of the second connector in the patient's body. The installation tool can be configured to be manipulated from outside the patient's body while the second connector is positioned within the patient's body. The second connector can include a first mating element, e.g., a groove formed therein. The installation tool can include a second mating element, e.g., a protrusion, configured to releasably mate with the first mating element. The first and second mating elements can be configured to be mated together during insertion of the second connector into the patient's body, which can allow the installation tool to be manipulated, e.g., held and moved by hand, to insert the second connector into the patient's body. The first and second mating elements can be configured to be mated together when the second connector is located within the patient's body, which can allow the installation tool to be manipulated to desirably position the second connector within the patient's body. When the second connector is in a desired position, the installation tool can be removed from the second connector by releasing the first and second mating elements from mating engagement, e.g., by sliding the protrusion out of the groove. In some embodiments, the second connector can be inserted into the patient's body without using the installation tool, and the installation tool can be mated to the second connector already located in the patient's body.

In some embodiments, a surgical device configured to anchor a spinal rod can be configured to attach to a

[symbol]

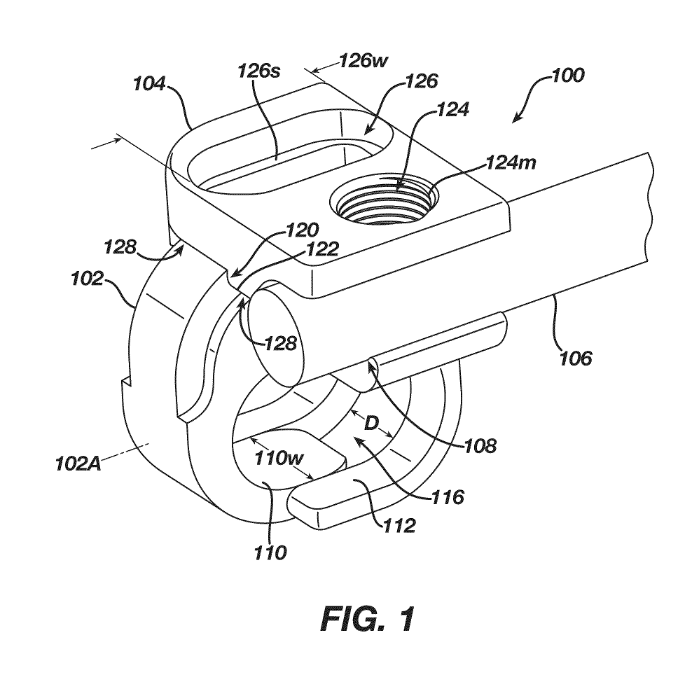

FIGS. 1-4 illustrate one embodiment of a surgical device 100, e.g., a bone connector, configured to attach to a rib. The device 100 can include a first connector 102 and a second connector 104 configured to couple to the first connector 102 and to be movable relative to the first connector 102. As in this illustrated embodiment, the first and second connectors 102, 104 can be discrete elements configured to be independently manipulated. The second connector 104 can include a rod seat 108 configured to seat an elongate rod 106, e.g., a spinal rod, therein. The rod seat 108 can be configured to movably seat the rod 106 therein, which can allow the rod 108 to be adjusted in position relative to the second connector 104 and, when the first and second connectors 102, 104 are coupled together, to the first connector 102. The first connector 102 can include a first seat 110 configured to seat a bone, e.g., a rib, therein. The second connector 104 can include a second seat 112 and a third seat 114 each configured to seat the bone therein. The first, second, and third seats 110, 112, 114 can cooperate to define a rib receiving area 116 of the device 100. As discussed further below, a size of the rib receiving area 116 can be adjusted by moving the second connector 104 relative to the first connector 102, which can allow the device 100 to accommodate ribs of various sizes and/or can allow the device 100 to be securely attached to a rib by adjusting the size of the rib receiving area 116 to correspond to the size of the rib to be seated in the first, second, and third seats 110, 112, 114.

The elongate rod 106 can have a variety of sizes, shapes, and configurations, as will be appreciated by a person skilled in the art. As in this illustrated embodiment, the elongate rod 106 can include a spinal rod and can have an elongate cylindrical shape.

The first connector 102 can have a variety of sizes, shapes, and configurations. As in this illustrated embodiment, the first connector 102 can include a C-shaped member having a first, top end region including a first bore 118 formed therethrough and a portion of a first engagement surface (obscured in FIGS. 1-4) formed thereon, a second, bottom end region including the first seat 110, and an intermediate region between the first and second end regions including a first mating surface 120 and including another portion of the first engagement surface. The first bore 118 can include a mating element (e.g., a thread, a snap fit member, a magnet, etc.) on an internal surface thereof, which can facilitate engagement of the first bore 118 with a surgical tool. The first bore 118 in this illustrated embodiment includes a mating element in the form of a thread in a lower region of the first bore 118, such that the thread is obscured from view in FIGS. 1-4.

The first bore 118 can be configured to selectively receive a surgical tool therein. The first bore's mating element can be configured to matingly engage the surgical tool. Unmating the surgical tool from the first bore 118 can disengage the surgical tool and the first connector 102 so as to allow release of the surgical tool from the first connector 102. For example, the surgical tool can include an installation tool (not shown) configured to engage the first mating element, e.g., threadably engage the first bore's thread, such as with a corresponding thread on a distal end of an elongate shaft of the installation tool. The installation tool engaged with the first connector 102 via the first bore 118 can be configured to be manipulated from outside a patient's body to insert and position the first connector 102 within the patient's body. The installation tool can be configured to be disengaged from the first bore 118, e.g., unthreaded therefrom, so as to allow removal of the installation tool from the patient's body and allow implantation of the first connector 102 within a patient's body without the installation tool connected thereto. For another example, the surgical tool can include a locking member (not shown), e.g., a set screw, a magnetic plug, a snap fit member, etc. The locking member can be configured to engage the second connector 104, e.g., abut against a surface thereof, when the first and second connectors 102, 104 are coupled together so as to lock the first and second connectors 102, 104 in a fixed position relative to one another. The locking member can be configured to be disengaged from the mating element, e.g., unscrewed from the first bore's thread, which can allow for the relative positions of the first and second connectors 102, 104 to be adjusted if the position in which the first and second connectors 102, 104 were locked together using the locking element is desired to be changed. In an exemplary embodiment, the first bore 118 can be configured to selectively receive an installation tool therein and a locking member therein, which can allow the installation tool to install the first connector 102 and then the locking member to lock the first and second connectors 102, 104 together.

The first engagement surface can include an external surface of the first connector 102. The first engagement surface can include a single surface or, as in this illustrated embodiment, can include a plurality of surfaces. The plurality of surfaces forming the first engagement surface in this illustrated embodiment include three surfaces that together define at least a portion of an outer surface of the first connector's top end region and an outer surface of the first connector's intermediate region.

The first seat 110 can include an arcuate member, as in this illustrated embodiment. The arcuate member can have a curved shape generally corresponding to a curved shape of a typical rib's exterior surface. The first seat 110 can thus be configured to securely engage a rib against an interior surface of the first seat 110.

The first mating surface 120 in the intermediate region of the first connector 102 can be configured to movably seat the second connector 104. The first mating surface 120 can have a curved shape configured to movably seat thereon a second mating surface 122 of the second connector 104. The first and second mating surfaces 120, 122 can be configured to be engaged with one another by being in contact with one another so as to mate the first and second connectors 102, 104 together. The first and second connectors 102, 104 do not need any other connection in order to be mated to one another for proper use. The first and second connectors 102, 104 can thus be easily connected together by placing the first and second mating surfaces 120, 122 in contact with one another. The first mating surface 120 can define a pivot point about which the second connector 104 having its second mating surface 122 engaged with the first mating surface 120 can pivot relative to the first connector 102, as discussed further below.

The second connector 104 can have a variety of sizes, shapes, and configurations. As in this illustrated embodiment, the second connector 104 can include a first, top end region including a second engagement surface 128, a second, bottom end region including the second and third seats 112, 114, and an intermediate region between the second connector's first and second end regions including the second mating surface 122. The first, top end region can include a second bore 124 formed therethrough, an opening 126 formed therethrough, the rod seat 108 formed therein that can be configured to seat the rod 106 therein, and a second engagement surface 128 configured to engage the first connector's first engagement surface.

The second bore 124 can include a second mating element 124m (e.g., a thread, a snap fit member, a magnet, etc.) on an internal surface thereof, which can facilitate engagement of the second bore 124 with a surgical tool. The second mating element 124m in this illustrated embodiment is in the form of a thread. The second bore 124 can be positioned adjacent to and be in communication with the rod seat 108, as in this illustrated embodiment, which can allow a surgical tool received in the second bore 124 to engage the rod 106 seated in the rod seat 108. Unmating the surgical tool from the second bore 124, e.g., unthreading the surgical tool from the second mating element 124m, can disengage the surgical tool and the second connector 104 so as to allow release of the surgical tool from the second connector 104. Examples of the surgical tool that can be configured to mate with the second bore 124 include an installation tool (not shown) and a locking member (not shown), similar to that discussed above regarding the first bore 118 of the first connector 102. Namely, an installation tool can be configured to engage the second mating element 124m to facilitate installation of the second connector 104, and a locking member can be configured to engage the second mating element 124m to facilitate a locking action. In the case of a locking member seated in the second bore 124, the locking member can be configured to engage the rod 106 seated in the rod seat 108 so as to lock the rod 106 in a fixed position relative to the second connector 104. When the first and second connectors 102, 104 are fixed in position relative to one another, e.g., by the locking member locking the first and second connectors 102, 104 in a fixed position relative to one another, the locking member seated in the second bore 124 can lock the rod 106 in a fixed position relative to both the first and second connectors 102, 104.

The rod seat 108 can have a C-shape so as to include a curved surface generally corresponding to a curved shape of a typical rod's exterior surface. The C-shape of the rod seat 108 can allow a longitudinal length of the rod 106 to be loaded therein. The opening of the C-shape can be on a side of the second connector 104, as in this illustrated embodiment, so as to allow the rod 106 to be side-loaded into the rod seat 108.

The rod seat 108 can be configured to movably seat the rod 106 therein. As in this illustrated embodiment, the rod 106 seated in the rod seat 108 can be configured to move longitudinally relative to the second connector 104, e.g., slide longitudinally within the rod seat 108, and can be configured to rotate about a longitudinal axis 106A of the rod 106 when seated within the rod seat 108. To effect such movement, the rod 106 can be moved while the second connector 104 is held in a stable position, or the second connector 104 can be moved while the rod 106 is held in a stable position. The longitudinal sliding movement of the rod 106 seated in the rod seat 108 can be in both directions along its longitudinal axis 106A, e.g., in a direction toward the first connector 102 and in an opposite direction away from the first connector 102. The locking member being seated in the second bore 124 and engaging the rod 106 seated in the rod seat 108 can be configured to prevent movement of the rod 106 relative to the second connector 104 so as to lock the rod 106 in position within the rod seat 108.

The opening 126 formed through the second connector 104 can be configured to receive a surgical tool therein. The opening 126 can be configured to align with the first bore 118 formed through the first connector 102 when the first and second connectors 102, 104 are mated together, e.g., when the first and second mating surfaces 120, 122 are mated to one another. In this way, the same surgical tool can be simultaneously received in the opening 126 and the first bore 118. As in the illustrated embodiment, the opening 126 can have an elongate shape that has a width 126w greater than a width 118w (e.g., a diameter) of the first bore 118, which can allow the first bore 118 to be aligned with the opening 126 at a variety of positions along the opening's width 126w.

As in this illustrated embodiment, the opening 126 can include a recessed shelf 126s configured to seat thereon a surgical tool inserted into the opening 126. The recessed shelf 126s can extend around a full perimeter of the opening 126, as in this illustrated embodiment. Alternatively, the recessed shelf 126s can extend around a partial perimeter of the opening 126 and/or can include a plurality of recessed shelves each extending around a partial perimeter of the opening 126. The recessed shelf 126s can be configured to prevent an enlarged portion of the surgical tool from passing entirely through the opening 126, which can help properly position the surgical tool within the opening 126. For example, the recessed shelf 126s can be configured to seat thereon a head of a set screw (not shown) that has a diameter greater than a diameter of a shank extending distally from the head and extending through the opening 126 into the first bore 118 aligned with the opening 126.

The second and third seats 112, 114 can each include an arcuate member, as in this illustrated embodiment. The arcuate members can, similar to the first seat 110, have a curved shape generally corresponding to a curved shape of a typical rib's exterior surface. The second and third seats 112, 114 can thus be configured to securely engage a rib against interior surfaces thereof.

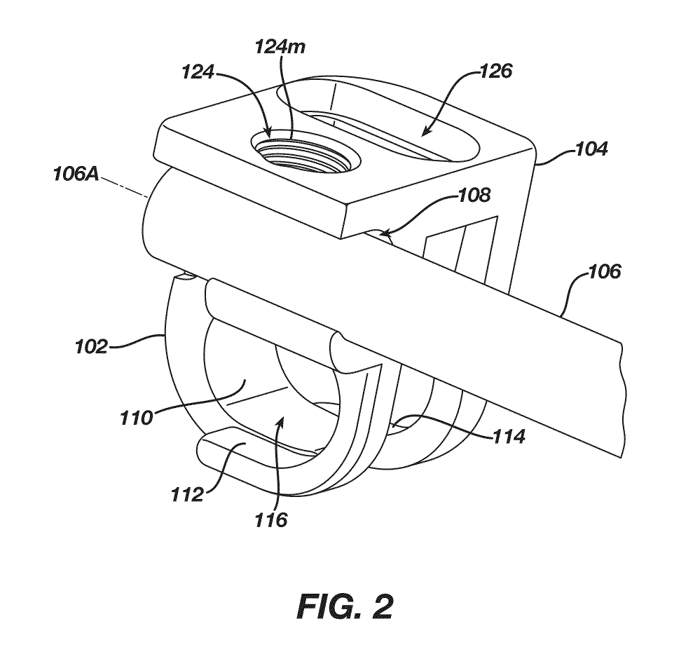

When the first and second connectors 102, 104 are mated together, the first, second, and third seats 110, 112, 114 can be configured to be axially arranged along a bone (e.g., a rib) seated in the first, second, and third seats 110, 112, 114, as shown for example in FIGS. 1 and 2. The device 100 can thus be configured to have three points of contact with the bone, one at each of first, second, and third seats 110, 112, 114. Being located at different axial positions along the rib can help distribute a load on the bone, thereby helping to prevent damage to the bone and/or to the device 100. The second and third seats 112, 114 can be separated from one another by a distance D that is large enough to allow the first seat 110 to be positioned between the second and third seats 112, 114, as shown for example in FIGS. 1 and 2, when the first and second connectors 102, 104 are mated together. In other words, the first seat 110 can have a width 110w that is equal to or less than the distance D between the second and third seats 112, 114. The first seat 110 being positioned between the second and third seats 112, 114 can help the device 100 have three points of contact with a bone engaged thereby and/or can keep the first connector 102 in a stable position relative to the second connector 104 before and after the first and second connectors 102, 104 are locked in position relative to one another, e.g., with a locking members seated in the opening 126 and the first bore 118.

The second mating surface 122 in the intermediate region of the second connector 104 can be configured to movably mate to the first mating surface 120 of the first connector 102. The second mating surface 122 can thus have a curved shape complementing the curved shape of the first mating surface 120 to facilitate the mating of the surfaces 120, 122.

As mentioned above, when the first and second connectors 102, 104 are coupled together, the second connector 104 can be configured to be movable relative to the first connector 102 to facilitate positioning of the second connector 104 relative to the first connector 102. This movement can include rotational movement and/or longitudinal sliding movement. In an exemplary embodiment, the second connector 104, when coupled to the first connector 102, can be configured to rotate relative to the first connector 102 and to longitudinally slide relative to the first connector 102 along a longitudinal axis 102A defined by the first connector 102.

The second connector 104 can be configured to rotate relative to the first connector 102 to adjust the second connector's position relative to the first connector 102 when the first and second mating surfaces 120, 122 are mated together by sliding the second mating surface 122 along the first mating surface 120. In other words, the second connector 104 can be rotated about the pivot point defined by the first mating surface 120 when the first and second mating surfaces 120, 122 are mated together to cause rotation of the second connector 104 relative to the first connector 102. The complementary curved shapes of the first and second mating surfaces 120, 122 can facilitate this rotational movement. The rotational movement of the second connector 104 can be in a first direction (e.g., clockwise) toward the first connector 102 so as to move the second and third seats 112, 114 toward the first seat 110, and can be in a second direction (e.g., counterclockwise) opposite to the first direction so as to move the second and third seats 112, 114 away from the first seat 110. The second connector 104 may only need to be rotated in the first direction to seat the bone in the second and third seats 112, 114. However, the second connector 104 can be configured to be alternately rotated in the first and second directions as many times as needed to seat a bone in a desired position in the second and third seats 112, 114. In some embodiments, the rod 106 can be seated and locked in the rod seat 108 prior to the rotation of the second connector 104 relative to the first connector 102 such that the rod 106 also rotates relative to the first connector 102. In other embodiments, the rod seat 108 can be empty of the rod 106 during the second connector's rotation relative to the first connector.

The second connector 104 can be configured to longitudinally slide relative to the first connector 102 to adjust the second connector's position relative to the first connector 102 by sliding the second connector's second engagement surface 128, e.g., an inner surface of the second connector 104, along the first connector's first engagement surface, e.g., an outer surface of the first connector 102.

Optionally, the device 100 can be provided as a kit including a plurality of first connectors and plurality of second connectors. Each of the first connectors can be configured to couple to at least one of the second connectors. Each of the first connectors can have a different size from one another, and each of the second connectors can have a different size from one another. The kit including differently sizes of first and second connectors can facilitate use of the first and second connectors with a variety of different patients having differently sized bones and/or can facilitate use of the first and second connectors with fused ribs that have a larger size than a typical single rib. A medical professional, e.g., a surgeon, can thus use the kit during performance of a surgical procedure on a patient and can choose the most appropriately sized first and second connectors for the patient as observed during the surgical procedure.

In some embodiments, the kit can include a plurality of elongate rods each configured to couple to at least one of second connectors provided in the kit. The elongate rods can each have a different size from one another, e.g., a different longitudinal length and/or a different diameter, which can facilitate selection of an elongate rod during performance of a surgical procedure on a patient based on the patient's observed anatomy and/or on the positioning of devices to which the elongate rod is to be coupled within the patient's body.

As mentioned above, an elongate rod can be anchored using a first device, e.g., the device 100 of FIGS. 1-4, configured to attach to a first rib of a patient and using a second device configured to attach to a second rib of the patient such that the spinal rod can be anchored to the patient via the first and second ribs. In some embodiments, the second device can be identical to the first device. For example, the second device (not shown) can include a first connector and a second connector configured to couple to the first connector and to be movable relative to the first connector. One of the second device's first and second connectors can be configured to seat the elongate rod seated by the first device such that the elongate rod can span between the first bone to which the first device is attached and the second bone to which the second device is attached. In some embodiments, the second device can be different from the first device, such as by the first device being configured like the device 100 of FIGS. 1-4 and the second device being configured like another of the surgical devices described herein.

If the device 100 is provided as part of a kit, the kit optionally can include a plurality of first connectors and a plurality of second connectors for the first device, and can include a plurality of first connectors and a plurality of second connectors for the second device.

The surgical devices disclosed herein can be used to perform a surgical procedure in which an elongate rod, such as a spinal rod, is anchored within a patient's body. The surgical procedure can be a minimally invasive procedure or an open surgical procedure. The surgical devices disclosed herein can be used in robotic-assisted minimally invasive or open surgical procedures.

For example, a minimally invasive surgical procedure can begin by preparing the patient for surgery and making one or more appropriately sized incisions at a desired location. This general example is described with respect to the surgical device 100 of FIG. 1, but other embodiments of devices described herein can be similarly used. In a minimally invasive procedure, one or more access devices (not shown) can be positioned in the incision(s) to provide access to the surgical site. One or more viewing devices, e.g., scopes, can be placed in one of the incisions to allow medical personnel to view the surgical site from outside the body.

Once the patient is prepared for surgery, the surgical device 100 can be inserted through an incision and/or through an access device to the surgical site. The first and second connectors 102, 104 can be inserted sequentially into the body or can be inserted simultaneously into the body. In an exemplary embodiment, if the first and second connectors 102, 104 are inserted sequentially into the body, the first connector 102 can be inserted into the body before the second connector 104. As mentioned above, an installation tool can be mated to the first connector's first bore 118 to facilitate insertion of the first connector 102 into the body, and the same or a different installation tool can be mated to the second connector's second bore 124 to facilitate insertion of the second connector 104 into the body. In some embodiments, the rod 106 can be mated to the second connector 104 (e.g., locked thereto using a set screw seated in the second bore 124) and used as the installation tool for the second connector 104. Inserting the first and second connectors 102, 104 sequentially can allow the device 100 to be inserted into the body through a smaller incision than if the first and second connectors 102, 104 are inserted simultaneously into the body. In an exemplary embodiment, if the first and second connectors 102, 104 are inserted simultaneously into the body, an installation tool can be used to introduce both of the connectors 102, 104 into the body. For example, the installation tool can extend through the second connector's opening 126 and be mated to the first connector's first bore 118 aligned with the opening 126. For another example, the installation tool can include the rod 106 locked to the second connector 104 by, e.g., a set screw seated in the second bore 124. Inserting the first and second connectors 102, 104 simultaneously can allow the device 100 to be inserted with the first and second connectors 102, 104 coupled together, which can expedite positioning of the first and second connectors 102, 104 relative to a target bone (not shown) in the body. Inserting the first and second connectors 102, 104 simultaneously can provide more space at a surgical site to manipulate the first connector 102 relative to a target bone at the surgical site.

With the first connector 102 in the body, whether or not the second connector 104 is already in the body (e.g., because the second connector 104 was inserted simultaneously with the first connector 104), the first connector 102 can be manipulated to seat a target bone (e.g., a first rib) in the first connector's first seat 110. In some embodiments, seating the target bone in the first seat 110 can include rotating the first connector 102 such that the bottom end region of the first connector rotates around a top part of the target bone to hook the first seat 110 around the target bone. In some embodiments, seating the target bone in the first seat 110 can include moving the first connector 102 laterally relative to the bone so as to side-load the bone into the first seat 110. If the second connector 104 is not already in the body when the first seat 110 is seating the target bone, the second connector 104 can then be inserted into the body.

In an exemplary embodiment, the first and second connectors 102, 104 can be coupled together (e.g., by mating the first and second mating surfaces 120, 122) after the first seat 110 seats the target bone, which can provide more space for manipulating the first connector 102 to seat the target bone and/or can allow for sequential insertion of the first and second connectors 102, 104.

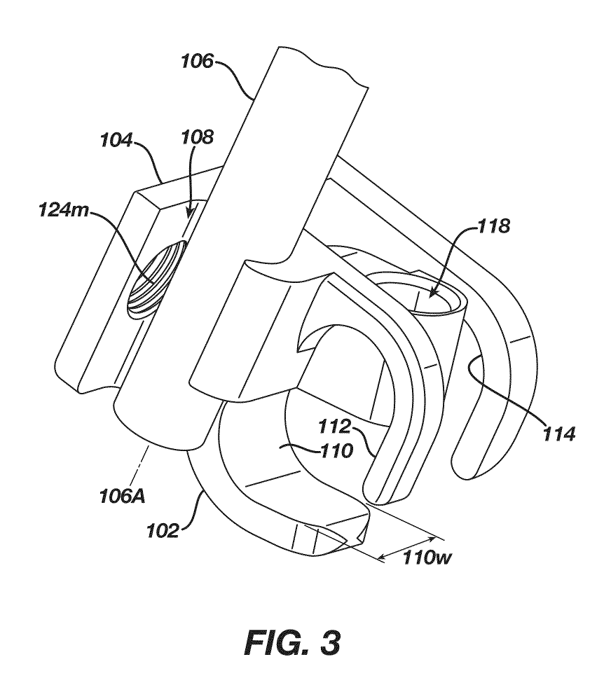

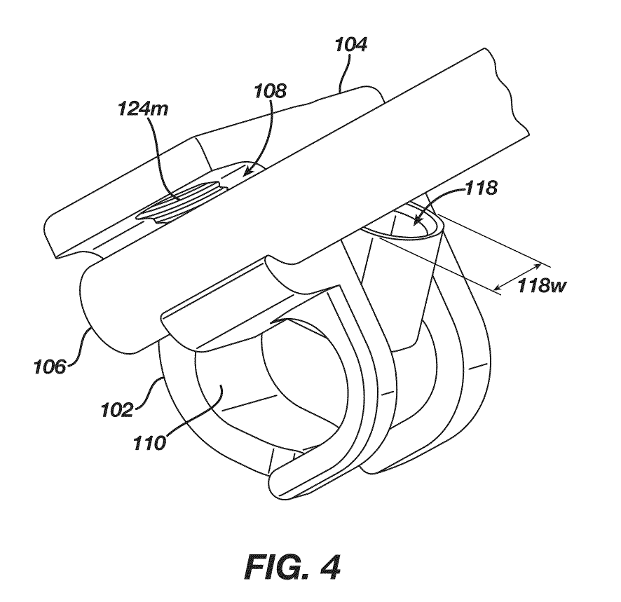

With the first seat 110 seating the target bone and the first and second mating surfaces 120, 122 mated together, as shown in FIG. 3, the second connector 104 can be rotated relative to the first connector 102 in the first direction such that the second and third seats 112, 114 move in a direction toward the first seat 110 and hence toward the target bone seated in the first seat 110, as shown in FIG. 4. The second connector 104 can be alternately rotated any number of times toward and away from the target bone in order to help desirably seat the target bone in the first, second, and third seats 110, 112, 114, thereby capturing the target bone in the rib receiving area 116.

The second connector 104 can be moved longitudinally relative to the first connector 102 in addition to being rotated relative to the first connector 102 in order to help desirably seat the target bone in the first, second, and third seats 110, 112, 114. In some embodiments, longitudinal movement may be unnecessary because, e.g., the size of the target bone does not require longitudinal sliding movement in order to desirably seat the target bone in the first, second, and third seats 110, 112, 114.

FIGS. 1 and 2 illustrate an example of a final position of the first and second connectors 102, 104 relative to one another in which the target bone can be seated in the first, second, and third seats 110, 112, 114. In an exemplary embodiment, the first, second, and third seats 110, 112, 114 each contact the target bone when the first and second connectors 102, 104 are in the final position. However, in some embodiments, the target bone may not contact all of the first, second, and third seats 110, 112, 114 when the first and second connectors 102, 104 are in the final position because of one or more irregularities, such as an inconsistency in the target bone's diameter, the target bone including multiple ribs fused together so as to form an irregularly size target bone, etc.

With the first and second connectors 102, 104 in a desired position relative to the target bone, e.g., in the final position of FIGS. 1 and 2, the first and second connectors 102, 104 can be locked in position relative to one another. As mentioned above, a locking member can be inserted into the opening 126 of the second connector 104 and the first bore 118 of the first connector 102 to lock the first and second connectors 102, 104 together. The elongate shape of the opening 126 can compensate for the first bore 118 being aligned therewith at different points along the width 126w of the opening 126 depending on how the first and second connectors 102, 104 are positioned relative to one another for the patient's specific anatomy. For example, a larger diameter target bone can cause the first bore 118 to be aligned with the opening 126 toward one end thereof, while a smaller diameter target bone can cause the first bore 118 to be aligned with the opening 126 toward an opposite end thereof.

If the rod 106 has not already been inserted into and locked within the rod seat 108, the rod 106 can be inserted into the rod seat 108 and locked therein by, e.g., inserting a locking member into the second bore 124 until the locking member engages the rod 106 so as to press on and lock the rod 106 in place.

As mentioned above, a second device can be connected to a second target bone (e.g., a second rib), and the rod 106 coupled to the device 100 can be coupled to the second device so as to span between the target bone and the second target bone.

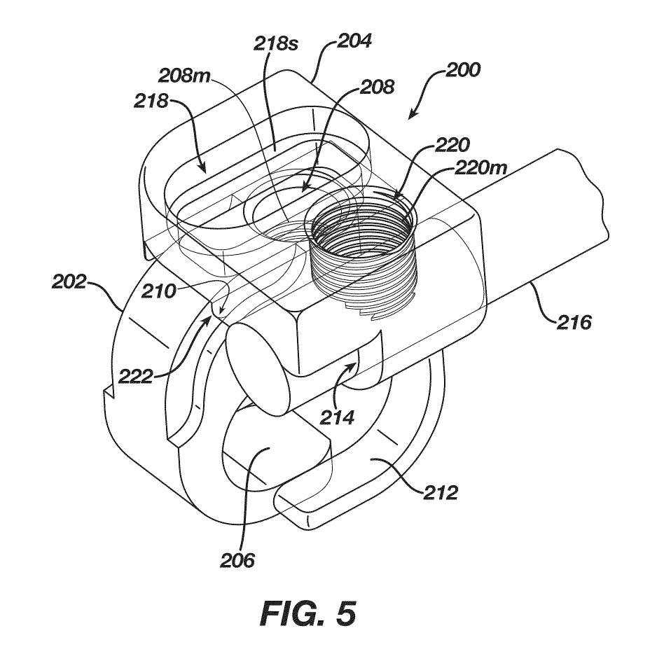

FIG. 5 illustrates another embodiment of a surgical device 200, e.g., a bone connector, configured to attach to a rib. The device 200 can generally be configured and used similar to the device 100 of FIGS. 1-4. The device 200 in this illustrated embodiment includes a first connector 202 that includes a first seat 206 configured to seat a bone (e.g., a rib) therein, that has a first bore 208 formed therethrough and including a mating element 208m, and that includes a first mating surface 210. The first bore's mating element 208m in this illustrated embodiment includes a thread. The device 200 in this illustrated embodiment also includes a second connector 204 configured to couple to the first connector 202 and to be movable relative to the first connector 202. The second connector 204 can include a second seat 212 and a third seat (obscured in FIG. 5) each configured to seat the bone therein, a rod seat 214 configured to seat an elongate rod 216, an opening 218 formed therethrough and including a recessed shelf 218s, a second bore 220 formed therethrough and including a second mating element 220m, and a second mating surface 222 configured to engage the first connector's first mating surface 210. The second bore's mating element 220m in this illustrated embodiment includes a thread.

Unlike the C-shaped rod seat 108 of the device 100 of FIG. 1, the rod seat 214 of the device 200 of the embodiment of FIG. 5 can have an enclosed 0-shape so as to include an enclosed cylindrical surface generally corresponding to a cylindrical shape of a typical rod's exterior surface. The rod seat 214 can thus be configured to have the rod 216 end-loaded therein and slide within the rod seat 214.

In the embodiments of FIGS. 1-5, the movement of the elongate rod mated to the surgical device includes sliding movement of the rod along respective longitudinal axes of the elongate rod, and includes rotational movement of the rod about its longitudinal axes. In other embodiments, an elongate rod mated to a surgical device can be configured to pivot relative to the device about a pivot point defined by a connection point between the rod and the device. This pivoting movement can be monoaxial, e.g., limited to one axis of motion about the pivot point, or this pivoting movement can be polyaxial, e.g., unlimited range of motion about the pivot point. The rod being pivotable relative to the device to which it is mated can facilitate positioning of the rod relative to a target treatment site.

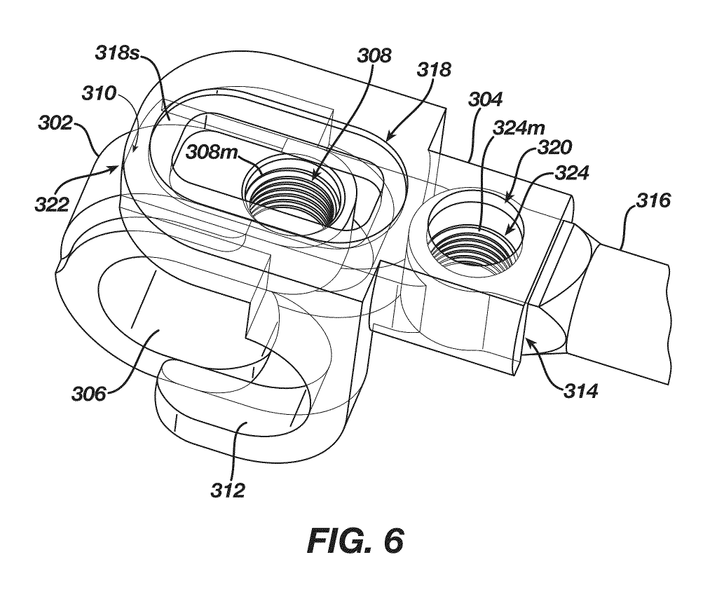

FIG. 6 illustrates another embodiment of a surgical device 300, e.g., a bone connector, configured to attach to a rib. The device 300 in this illustrated embodiment can be configured to allow an elongate rod 316 mated thereto to be polyaxially pivotable relative thereto.

The device 300 can generally be configured and used similar to the device 100 of FIGS. 1-4. The device 300 in this illustrated embodiment includes a first connector 302 that includes a first seat 306 configured to seat a bone (e.g., a rib) therein, that has a first bore 308 formed therethrough and including a mating element 308m, and that includes a first mating surface 310. The first bore's mating element 308m in this illustrated embodiment includes a thread. The device 300 in this illustrated embodiment also includes a second connector 304 configured to couple to the first connector 302 and to be movable relative to the first connector 302. The second connector 304 can include a second seat 312 and a third seat (obscured in FIG. 5) each configured to seat the bone therein, a rod seat 314 configured to seat an elongate rod 316, an opening 318 formed therethrough and including a recessed shelf 318s, a second bore 320 formed therethrough, and a second mating surface 322 configured to engage the first connector's first mating surface 310. The second bore 320 in this illustrated embodiment is unthreaded.

The second bore 320 can be positioned adjacent to and be in communication with the rod seat 314, as in this illustrated embodiment. The rod 316 can include a bore 324 formed therein that can be configured to be positioned in the rod seat 314 and aligned with the second bore 320 when the rod 316 is seated in the rod seat 314, as illustrated in FIG. 6. One or both terminal ends of the rod 316 can have a bore 324 formed therethrough. In an exemplary embodiment, both terminal ends of the rod 316 can have a bore 324 formed therethrough, which can allow the rod 316 to be polyaxially movable relative to the device 300 attached to one terminal end of the rod 316 and to be polyaxially movable relative to a second device (not shown) attached to the other terminal end of the rod 316.

The rod 316 can be pre-loaded into the rod seat 314. Pre-loading of the rod 316 into the rod seat 314 can help speed use of the device 300 and/or can help ensure that a correctly sized rod is inserted into the rod seat 314. Alternatively, the rod 316 can be inserted into the rod seat 314 during performance of a surgical procedure, which can provide medical personnel with flexibility in choosing a particular rod for a particular patient.

The rod's bore 324 can include a mating element 324m (e.g., a thread, a snap fit member, a magnet, etc.) on an internal surface thereof, which can facilitate engagement of the rod's bore 324 with a surgical tool (e.g., an installation tool, a set screw, etc.). In this illustrated embodiment, the rod's mating element 324m includes a thread. In this way, a surgical tool can be mated to the rod's mating element 324m to secure the tool to the rod 316 and to the second connector 304. For example, when the rod 316 is in a desired position relative to the second connector 304, a set screw (not shown) can be inserted into the second connector's second bore 320 and into the rod's bore 324 to mate with the mating element 324m and thereby be secured within the bores 320, 324 so as to lock the rod 316 in position relative to the second connector 304. For another example, an installation tool (not shown) can be inserted into the second connector's second bore 320 and into the rod's bore 324 to mate with the mating element 324m and thereby be secured within the bores 320, 324 so as to allow the installation tool to be manipulated to move the second connector 304 and the rod 316 as a unit. For yet another example, when the rod 316 is not seated in the rod seat 314, an installation tool (not shown) can be inserted into the rod's bore 324 to mate with the mating element 324m and thereby be secured within the bore 324 so as to allow the installation tool to be manipulated to move the rod 316.

When the rod 316 is seated in the rod seat 314 of the device 300, the rod 316 can be configured to be polyaxially pivotable relative to the second connector 304. The rod 316 can be configured to be polyaxially movable about a pivot point defined by a connection point between the rod 316 and the second connector 304, e.g., about a point defined by a coaxial longitudinal axis of the bores 320, 324.

In the embodiments of FIGS. 1-6, the second connector that includes two seats configured to seat a bone also includes a rod seat configured to seat an elongate rod. In other embodiments, a first connector that includes a single seat configured to seat a bone can include a rod seat configured to seat an elongate rod.

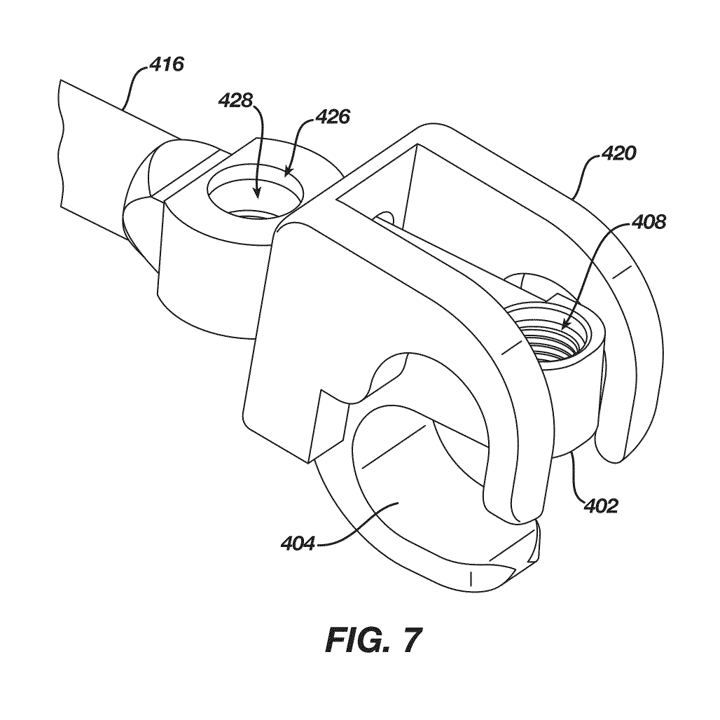

FIGS. 7-11 illustrate an embodiment of a surgical device 400, e.g., a bone connector, configured to attach to a rib. The device 400 in this illustrated embodiment includes a first connector 402 that includes a single seat 404 configured to seat a bone (e.g., a rib) and that includes a rod seat 406 configured to seat an elongate rod 416.

The device 400 can generally be configured and used similar to the device 100 of FIGS. 1-4. The device 400 in this illustrated embodiment includes the first connector 402 that includes the first seat 404, that has a first bore 408 formed therethrough and including a first mating element 408m, that includes a first mating surface 410, and that includes a second bore 426 formed therethrough. The first bore's mating element 408m in this illustrated embodiment includes a thread. The device 400 in this illustrated embodiment also includes a second connector 420 configured to couple to the first connector 402 and to be movable relative to the first connector 402. The second connector 420 can include a second seat 412 and a third seat 424 each configured to seat the bone therein, an opening 418 formed therethrough and including a recessed shelf 418s, and a second mating surface (obscured in FIGS. 7-11) configured to engage the first connector's first mating surface 410.

In general, the second bore 426 formed in the first connector 302 can be configured and used similar to the second bore 320 formed in the second connector 304 of the embodiment in FIG. 6, and the rod 416 can be configured and used similar to the rod 316 of the embodiment in FIG. 6. When the rod 416 is seated in the rod seat 406 of the device 400, the rod 416 can be configured to be polyaxially pivotable relative to the first connector 402. The rod 416 can be configured to be polyaxially movable about a pivot point defined by a connection point between the rod 416 and the first connector 402, e.g., about a point defined by a coaxial longitudinal axis of the second bore 426 and a bore 428 formed through an end of the rod 416. The rod's bore 428 can include a mating element 428m (e.g., a thread, a snap fit member, a magnet, etc.) on an internal surface thereof, which can facilitate engagement of the rod's bore 428 with a surgical tool (e.g., an installation tool, a set screw, etc.). In this illustrated embodiment, the rod's mating element 428m includes a thread.

The opening 418 formed through the second connector 420 in this illustrated embodiment is an open shape, unlike the openings 126, 218, 318 in the embodiments of FIGS. 1, 5, and 6, respectively, that have closed shapes. In other words, the openings 126, 218, 318 have closed perimeters, while the opening 418 has an open perimeter. The open shape of the opening 418 can be configured to facilitate rotation of the second connector 420 relative to the first connector 402, as discussed further below.

One embodiment of movement of the device 400 to seat a bone (e.g., a rib) in the first, second, and third seats 404, 412, 424 is illustrated in FIGS. 7-11. The movement of the device 400 can generally be similar to that discussed above regarding the movement of the device 100 of FIGS. 1-4.

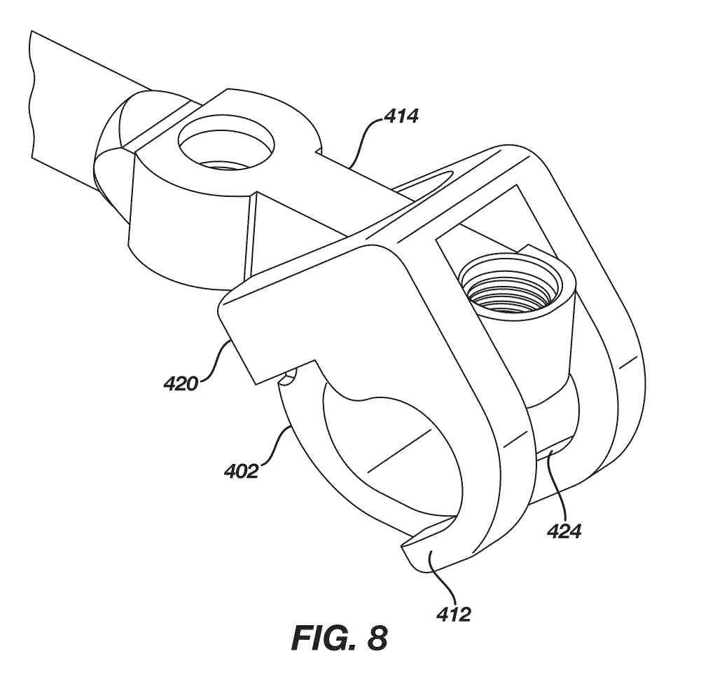

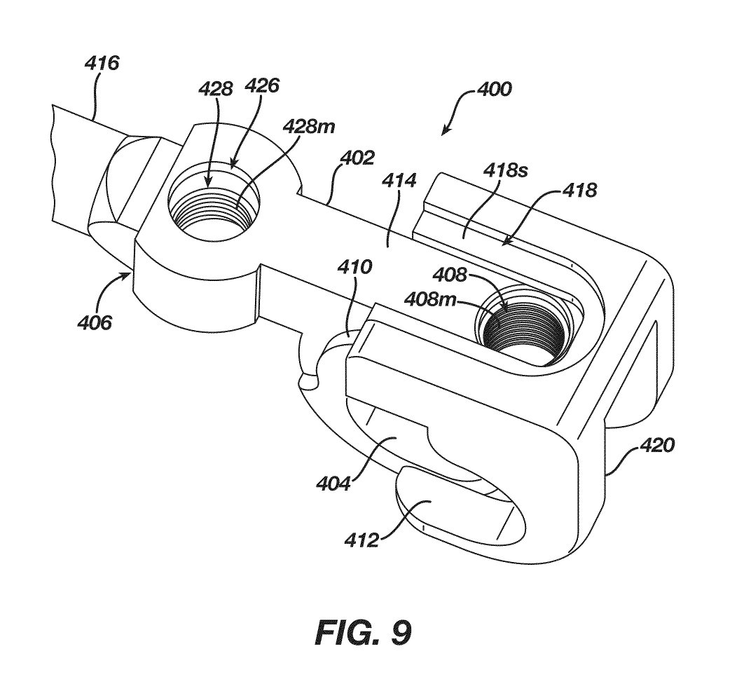

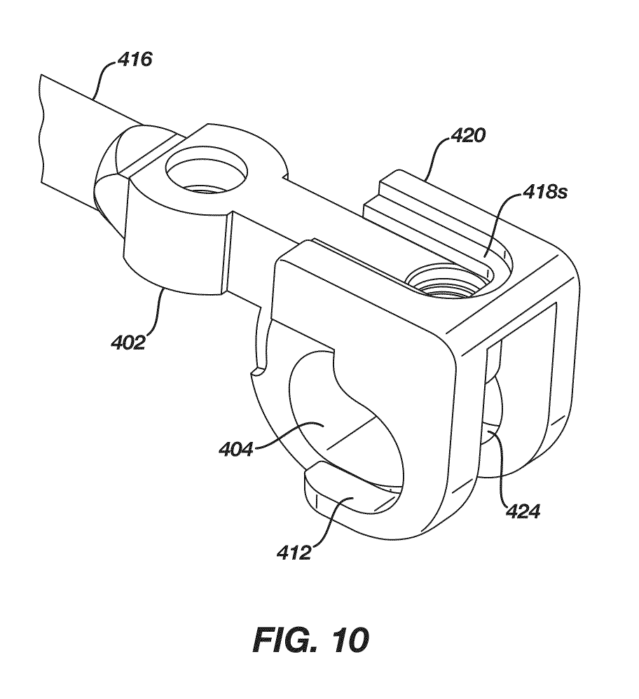

With the first connector 402 in the body, whether or not the second connector 420 is already in the body, the first connector 402 can be manipulated to seat a target bone (e.g., a first rib) in the first connector's first seat 412. With the first seat 404 seating the target bone and the first mating surface 410 mated with the second mating surface, as shown in FIG. 7, the second connector 420 can be rotated relative to the first connector 402 in the first direction such that the second and third seats 412, 424 move in a direction toward the first seat 404 and hence toward the target bone seated in the first seat 410, as shown in FIG. 8. As shown in FIGS. 7 and 8, the second connector's opening 418 can be configured to facilitate the second connector's rotation by allowing an elongate connector portion 414 of the first connector 402 to be seated within the opening 418 during the second connector's rotation, thereby increasing a range of the second connector's possible rotation and/or helping to reduce an amount of space needed for the second connector 420 to rotate. As in this illustrated embodiment, the second connector 420 can be configured to have a range of rotation of about 90°, as shown from FIG. 7 to FIG. 9. FIG. 10 illustrates the device 400 of FIG. 9 from another perspective. A person skilled in the art will appreciate that the range of rotation may not be precisely 90° due to, e.g., manufacturing tolerances, but be nevertheless considered to be about 90°. The second connector 420 can be alternately rotated any number of times toward and away from the target bone in order to help desirably seat the target bone in the first, second, and third seats 404, 412, 424.

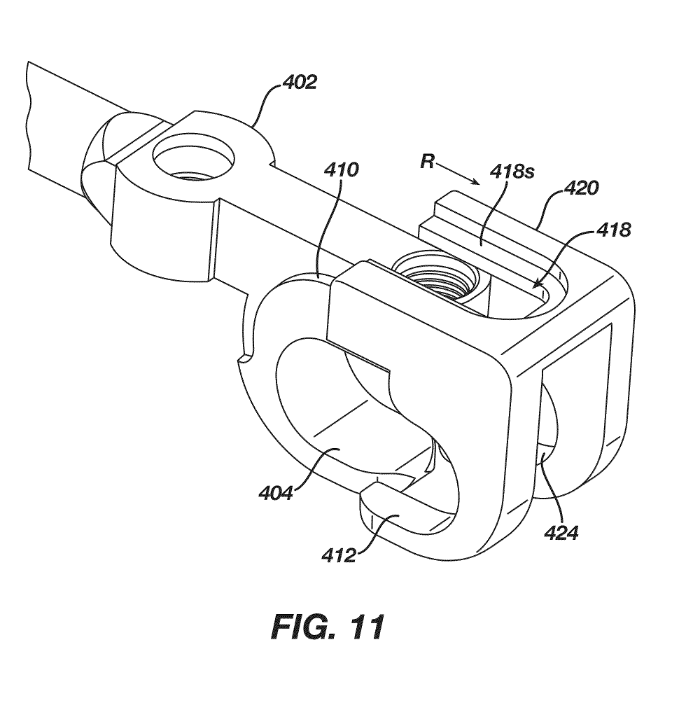

The second connector 420 can be moved longitudinally relative to the first connector 402 in addition to being rotated relative to the first connector 402 in order to help desirably seat the target bone in the first, second, and third seats 404, 412, 424. FIG. 11 shows the second connector 420 longitudinally slid from its position in FIGS. 9 and 10 in a direction R away from the first connector 402. As mentioned above, this longitudinal sliding can help the device 400 accommodate different sizes of bone in the seats 404, 412, 424. The second connector 420 can also be configured to longitudinally slide relative to the first connector 402 in a direction opposite to the direction R to facilitate seating of the bone.

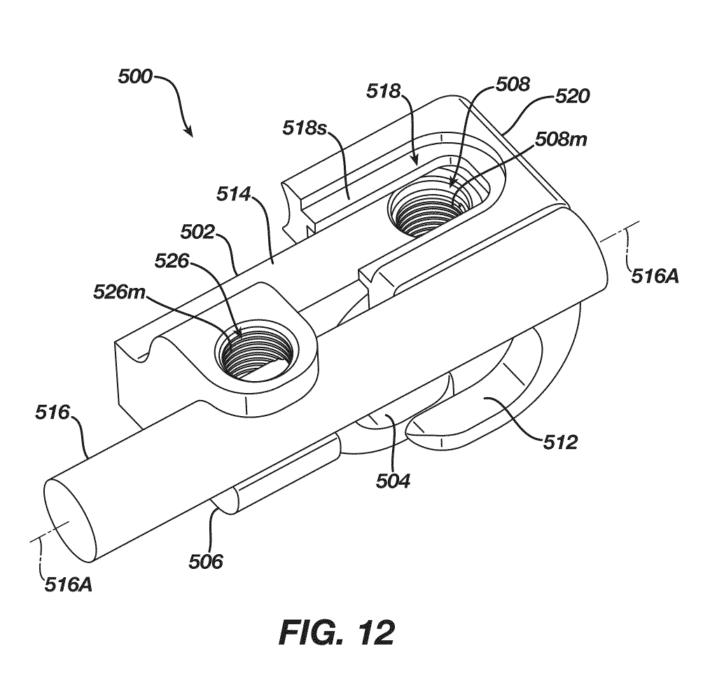

FIG. 12 illustrates another embodiment of a surgical device 500 including a first connector 502 that includes a single seat 504 configured to seat a bone (e.g., a rib) and that includes a rod seat 506 configured to seat an elongate rod 516. The device 500 can generally be configured and used similar to the device 100 of FIGS. 1-4. The device 500 in this illustrated embodiment includes the first connector 502 that includes the first seat 504, that has a first bore 508 formed therethrough and including a first mating element 508m, that includes a first mating surface 510, and that includes a second bore 526 formed therethrough and including a second mating element 526m. The first bore's first and second mating elements 508m, 526m in this illustrated embodiment each include a thread. The device 500 in this illustrated embodiment also includes a second connector 520 configured to couple to the first connector 502 and to be movable relative to the first connector 502. The second connector 520 can include a second seat 512 and a third seat (obscured in FIG. 12) each configured to seat the bone therein, an opening 518 formed therethrough and including a recessed shelf 518s, and a second mating surface (obscured in FIG. 12) configured to engage the first connector's first mating surface 510.

In general, the rod seat 506 and the second bore 526 of the first connector 502 can be configured and used similar to the rod seat 108 and the second bore 124 of the second connector 104 of FIGS. 1-4. When the rod 516 is seated in the rod seat 506, e.g., side-loaded therein, the rod 516 can be configured to move longitudinally relative to the first connector 502, e.g., slide longitudinally within the rod seat 506, and can be configured to rotate about a longitudinal axis 516A of the rod 516 when seated within the rod seat 506.

In general, the second connector 520 can be configured to move relative to the first connector 502 similar to the movement of the second connector 420 of FIGS. 7-11 relative to the first connector 402. Similar to the opening 418 of the second connector 420, the opening 518 of the second connector 520 can have an open shape, and the second connector's opening 518 can be configured to facilitate the second connector's rotation by allowing an elongate connector portion 514 of the first connector 502 to be seated within the opening 518 during the second connector's rotation.

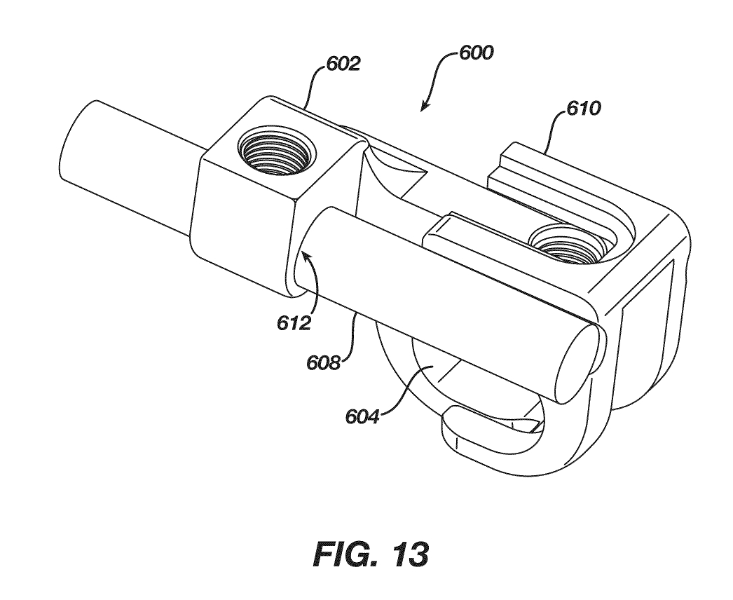

FIG. 13 illustrates another embodiment of a surgical device 600 including a first connector 602 that includes a single seat 604 configured to seat a bone (e.g., a rib) and that includes a rod seat 606 configured to seat an elongate rod 608. The device 600, which can also include a second connector 610, can generally be configured and used similar to the device 500 of the embodiment of FIG. 12. In contrast to the device 500, the device 600 in this illustrated embodiment includes a rod seat 612 having an enclosed 0-shape, similar to the rod seat 214 of the embodiment of FIG. 5. The rod seat 612 can thus be configured to have the rod 608 end-loaded therein and slide within the rod seat 612.

In the embodiments of FIGS. 1-13, a bone seated by a surgical device including first and second connectors can be configured to be seated by one arcuate extension of the first connector and two arcuate extensions of the second connector. In other embodiments, a surgical device including first and second connectors can be configured to seat a bone using a seat that is in the form of an arcuate extension and using a seat that is not in the form of an arcuate extension. Such a device can be configured to seat a bone more quickly during a surgical procedure than a device using arcuate extensions to seat a bone.

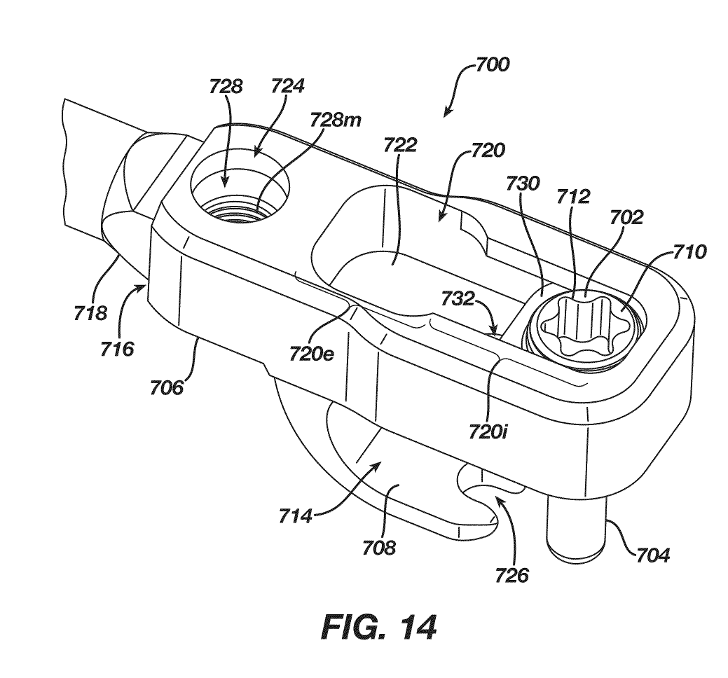

FIG. 14 illustrates another embodiment of a surgical device 700, e.g., a bone connector, configured to attach to a rib. The device 700 in this illustrated embodiment can include a first connector 702 including an elongate seat 704 configured to seat a bone thereagainst, and can include a second connector 706 configured to couple to the first connector 702 and including a second seat 708 in the form of an arcuate extension configured to seat the bone therein.

The device 700 can generally be configured and used similar to the device 100 of FIGS. 1-4. The device 700 in this illustrated embodiment includes the first connector 702 that includes the first seat 704. As in this illustrated embodiment, the first seat 704 can include an elongate post of the first connector 702 extending distally from a head 710 of the first connector 702. The head 710 can include a mating element 712 (e.g., a socket, a protrusions, etc.) configured to facilitate mating of the first connector 702 to an installation tool, e.g., a driver, and the head 710 can include a washer 730 configured to facilitate coupling of the first and second connectors 702, 706. As in this illustrated embodiment, the washer 730 can be integral with the first connector 702. The mating element 712 in this illustrated embodiment includes a socket formed in the head 710. The second connector 706 can include the second seat 708, a rod seat 716 configured to seat an elongate rod 718, an opening 720 formed therethrough and having a bottom surface 722, a second bore 724 formed therethrough, and a groove 726 formed therein and configured to receive the first connector 702, e.g., the elongate post 704, therein. As in this illustrated embodiment, the groove 726 can be formed in an end of the second seat 708 such that when the first connector 702 is seated therein, the first connector 702 faces the arcuate extension and defines a rod receiving area 714 therebetween.

The rod seat 716 and the rod 718 in this illustrated embodiment can generally be configured and used similar to the rod seat 314 and the rod 316 of the embodiment of FIG. 6. Namely, when the rod 718 is seated in the rod seat 716 of the device 700, the rod 718 can be configured to be polyaxially pivotable relative to the second connector 706. The rod 718 can be configured to be polyaxially movable about a pivot point defined by a connection point between the rod 718 the second connector 706, e.g., about a point defined by a coaxial longitudinal axis of the second bore 724 and a bore 728 formed through an end of the rod 718. The rod's bore 728 can include a mating element 728m (e.g., a thread, a snap fit member, a magnet, etc.) on an internal surface thereof, which can facilitate engagement of the rod's bore 728 with a surgical tool (e.g., an installation tool, a set screw, etc.). In this illustrated embodiment, the rod's mating element 728m includes a thread.

The washer 730 can have a diameter greater than a diameter of the first connector's elongate post 704 and less than a diameter of the head 710. The washer 730 can thus be configured to retain the first connector 702 within the opening 720. The washer 730 has a square shape in this illustrated embodiment, but the washer 730 can have other shapes. The washer 730 has a square shape in this illustrated embodiment, but the washer 730 can have other shapes. In an embodiment in which the washer 730 is not integral with the first connector 702, the washer 730 can include an opening (not shown) formed therethrough and configured to receive the first connector 702 therein. The opening formed through the washer 730 can have a diameter greater than a diameter of the first connector's elongate post 704 and less than a diameter of the head 710. The washer 730 can thus be configured to retain the first connector 702 therein, with the head 710 being unable to pass through the washer's opening. The washer's opening can include a mating element (not shown) configured to facilitate mating of the washer 730 with the first connector 702. The washer's mating element can be configured to secure the first connector 702 in a fixed position relative to the washer 730, which can facilitate simultaneous movement of the washer 730 and the first connector 702 within the opening 730 and/or can help prevent the first connector 702 from becoming unintentionally uncoupled from the second connector 706. The first connector 702 can include a corresponding mating element (not shown) configured to mate with the washer's mating element. For example, the washer's mating element can include a thread on an internal surface of the washer's opening, and the first connector's mating element can include a thread on an external surface thereof.

The second connector's opening 720 in this illustrated embodiment has a closed shape. The closed shape of the opening 720 can be configured to facilitate coupling of the first and second connectors 702, 706, as discussed further below. The opening 720 can include an enlarged diameter portion 720e having a diameter greater than a remaining portion 720r of the opening 720. The diameter of the enlarged diameter portion 720e can be greater than a diameter of the washer 730 such that the washer 730 can be configured to pass freely through the enlarged diameter portion 720e of the opening. The diameter of the remaining portion 720r of the opening 720 can be less than the diameter of the washer 730 such that the washer 730 cannot pass through the opening's remaining portion 720r. The opening 720 can thus be configured to allow passage of the washer 730 through one portion thereof, e.g., the enlarged diameter portion 720e, and to prevent passage of the washer 730 through another portion thereof, e.g., the remaining portion 720r. The washer 730 can thus be configured to be selectively retained within the opening 720, and hence the first connector 702 that includes the washer 730 can be configured to be selectively retained within the opening 720.

The washer 730, and hence the first connector 702, can be configured to be slidably movable within the opening 720. A bottom surface of the washer 730 can be configured to slidingly mate with the bottom surface 722 of the opening 720 when the washer 730 is disposed within the opening 720.

The groove 726 formed in the second connector 706 configured to receive the first connector 702 can be aligned with the remaining portion 720r of the opening, e.g., can be offset from the enlarged portion 720e. In this way, when the washer 730 is disposed in the opening 720 and the first connector 702 is seated in the groove 726, the washer 730 can be retained within the opening 720 by being located at least partially within the remaining portion 720r having a diameter too small to allow the washer 730 seated in the opening 720 to pass therethrough and hence help retain the first connector 702 within the groove 726.

The second connector 706 can include a secondary opening 732 extending therethrough. The secondary opening 732 can be in communication with the opening 720 and can be formed in the bottom surface of the opening 720. The secondary opening 732 can be configured to limit movement of the first connector 702 within the opening 720. The secondary opening 732 can be formed in an area of the second connector 706 aligned with the remaining portion 720r of the opening 720. The secondary opening 732 can thus be configured to prevent the washer 730 from being fully aligned with the opening's enlarged portion 720e, thereby preventing the washer 730 from passing out of the opening 730, and hence preventing the first connector 702 from falling out of the opening 720.

In an embodiment of using the device 700, with the second connector 706 in the body, whether or not the first connector 702 is already in the body, the second connector 706 can be manipulated to seat a target bone (e.g., a first rib) in the second connector's seat 708. In an exemplary embodiment, the first connector 702 is not coupled to the second connector 706 when the second connector 706 seats the target bone, which can facilitate side-loading of the bone into the second connector's seat 708. The first connector 702 can thus be mated to the second connector 706 after the target bone has been seated by the second connector's seat 708. In an embodiment in which the first connector 702 and the washer 730 are not integrally formed, the first connector 702 can be seated in the washer 730 before or after the washer 730 is disposed in the opening 720.

With the second connector's seat 708 seating the target bone and the first connector 702 coupled to the second connector 704, the first connector 702 can be moved relative to the second connector 708, e.g., by sliding laterally within the opening 720 and the secondary opening 732, such that the first connector's seat 704 moves in a direction toward the second connector's seat 708 and hence toward the target bone seated therein. In an exemplary embodiment, the first and second connectors 702, 706 chosen to use with the target bone have a size such that the first connector's post 704 is seated in the groove 726 when the post 704 and the second connector's seat 704 contact the target bone.

The rod 718 can be polyaxially adjusted relative to the second connector 706 and secured in position relative thereto using, e.g., a set screw, inserted into the bores 724, 728 and threadably engaged with the mating element 728m. In an exemplary embodiment, the rod 718 can be adjusted in position relative to second connector 706 after the target bone has been seated by the seats 704, 708, which can help ensure that the rod 718 is angled in a most effective direction for treatment.

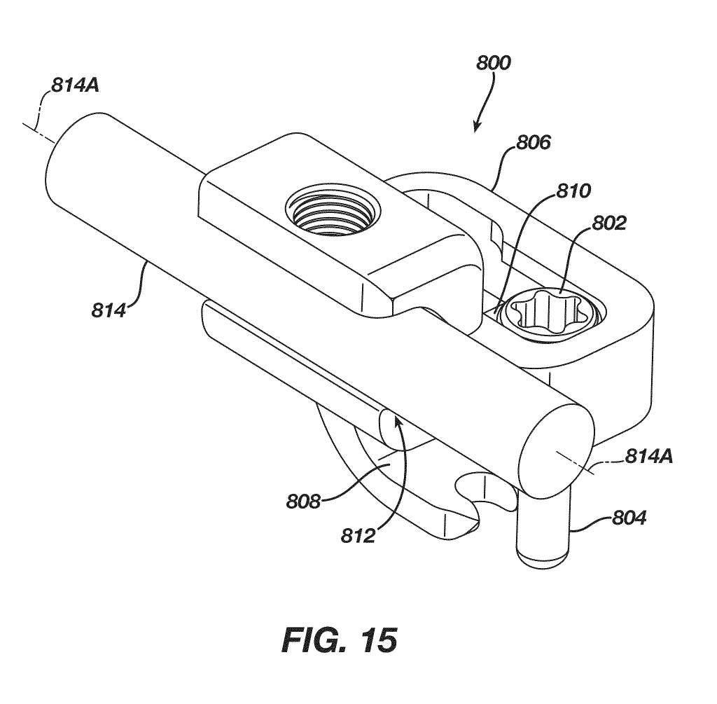

FIG. 15 illustrates another embodiment of a surgical device 800 including a first connector 802 including an elongate seat 804 configured to seat a bone (e.g., a rib) thereagainst, and a second connector 806 configured to couple to the first connector 802 and including a second seat 808 in the form of an arcuate extension configured to seat the bone therein. The device 800, which can also include a washer 810, can generally be configured and used similar to the device 700 of the embodiment of FIG. 14. In contrast to the device 700, the device 800 in this illustrated embodiment includes a rod seat 812 having a C-shape, similar to the rod seat 108 of the embodiment of FIG. 1. The rod seat 812 can thus be configured to have a rod 814 side-loaded therein, with the rod 814 seated therein being configured to move within the rod seat 812 relative to the second connector 806 by being rotated about a longitudinal axis 814A of the rod 814 and by longitudinally sliding within the rod seat 812.

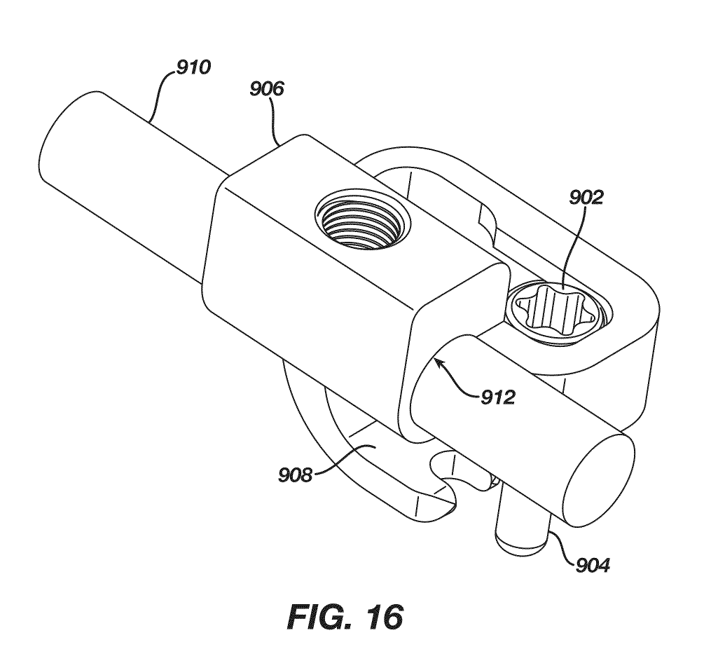

FIG. 16 illustrates another embodiment of a surgical device 900 including a first connector 902 including an elongate seat 904 configured to seat a bone (e.g., a rib) thereagainst, and a second connector 906 configured to couple to the first connector 902 and including a second seat 908 in the form of an arcuate extension configured to seat the bone therein. The device 900, which can also include a washer (obscured in FIG. 16), can generally be configured and used similar to the device 700 of the embodiment of FIG. 14. In contrast to the device 700 and to the device 800 of the embodiment of FIG. 15, the device 900 in this illustrated embodiment includes a rod seat 912 having an O-shape, similar to the rod seat 214 of the embodiment of FIG. 5. The rod seat 912 can thus be configured to have a rod 910 end-loaded therein and slide within the rod seat 912.

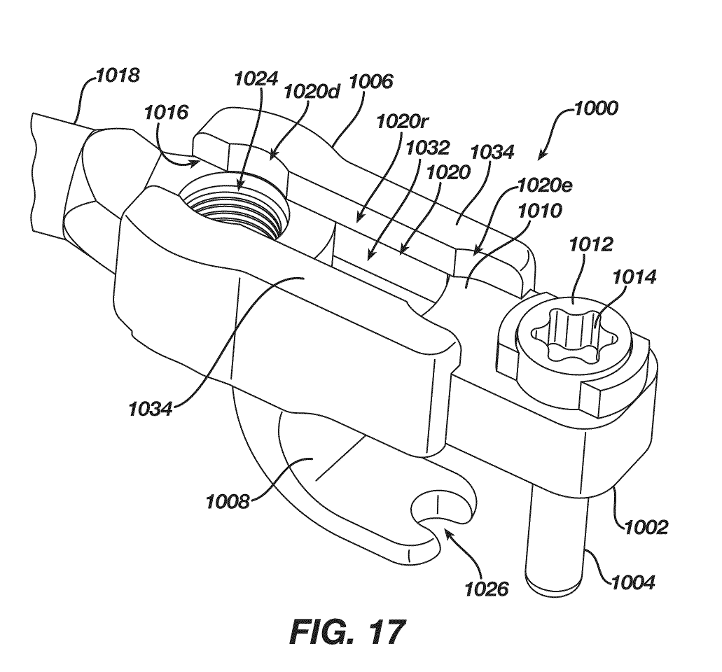

FIGS. 17-21 illustrate another embodiment of a surgical device 1000 including a first connector 1002 including an elongate seat 1004 configured to seat a bone (e.g., a rib) thereagainst, and a second connector 1006 configured to couple to the first connector 1002 and including a second seat 1008 in the form of an arcuate extension configured to seat the bone therein. The device 1000, which can also include a washer 1010, can generally be configured and used similar to the device 700 of the embodiment of FIG. 14. The elongate seat 1004 can include an elongate post of the first connector 1002 extending distally from a head 1012 of the first connector 1002. The head 1012 can include a mating element 1014 (e.g., a socket, a protrusions, etc.) configured to facilitate mating of the first connector 1002 to an installation tool, e.g., a driver, and the head 1012 can include the washer 1010. In this illustrated embodiment, the washer 1010 is integral with the first connector 1002. The second connector 1006 can include the second seat 1008, a rod seat 1016 configured to seat an elongate rod 1018, an opening 1020 formed therethrough and having a bottom surface 1030, and a groove 1026 formed therein configured to receive the first connector 1002, e.g., the elongate post 1004, therein.

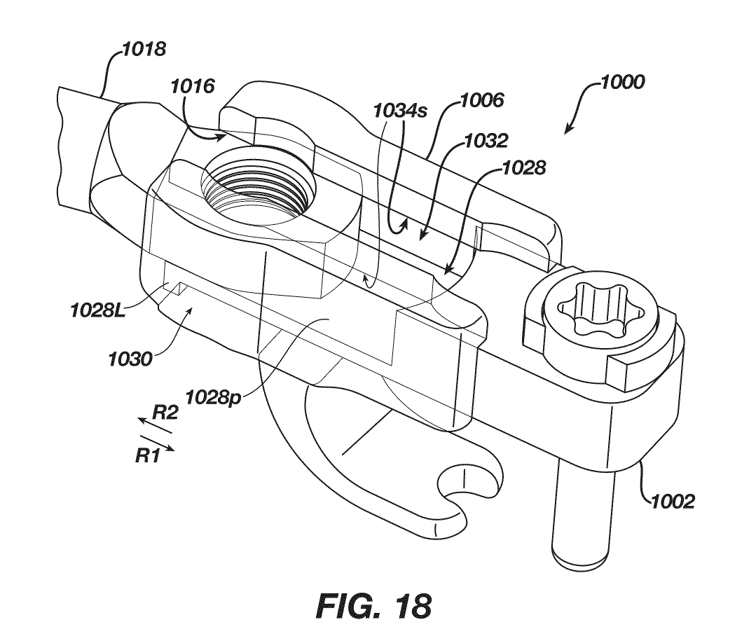

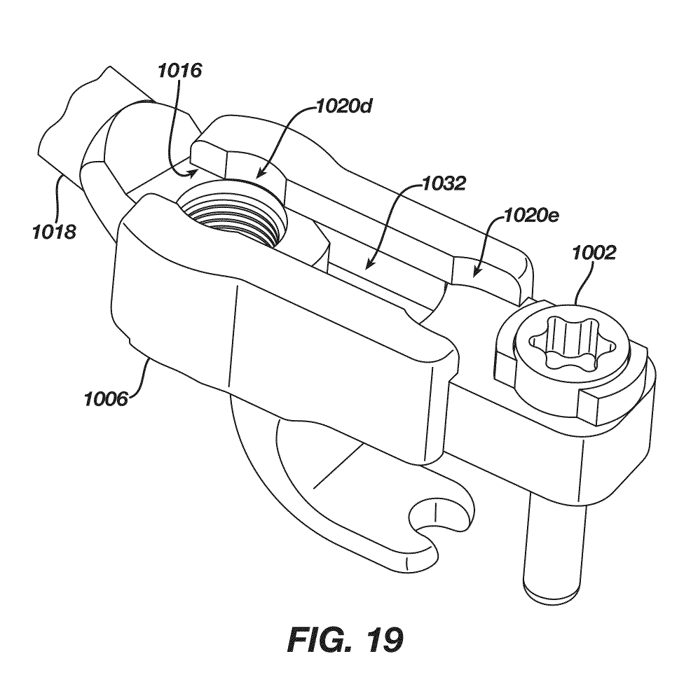

The rod seat 1016 and the rod 1018 in this illustrated embodiment can generally be configured and used similar to the rod seat 314 and the rod 316 of the embodiment of FIG. 6. FIGS. 17 and 18 illustrate the rod 1018 seated in the rod seat 1016 and at a first angular position relative to the second connector 1006. FIG. 19 illustrates the rod 1018 seated in the rod seat 1016 and at a second angular position relative to the second connector 1006 after the rod 1018 has been polyaxially adjusted relative to the second connector 1018.

The second connector's opening 1020 in this illustrated embodiment has an open shape. The opening 1020 can be configured to facilitate coupling of the first and second connectors 1002, 1006, as discussed further below. As in this illustrated embodiment, the opening 1020 can extend along a longitudinal length of the second connector 1006. Open ends of the opening 1020 can be located at opposed ends of the second connector 1006, with a first one of the open ends being configured to receive the rod 1018 therein and a second one of the open ends being configured to receive the first connector 1002 therein. The opening 1020 can thus be in communication with the rod seat 1016 and can be aligned therewith so as to be configured similar to the second bore 320 of the embodiment of FIG. 6 aligned with the rod seat 314. A surgical tool can thus be configured to be inserted into the opening 1020 and into a bore 1024 formed in the rod 1018. The first one of the open ends of the opening 1020 can include a first enlarged diameter portion 1020d, as in this illustrated embodiment, which can facilitate insertion of the surgical tool into the bore 1024 by not obscuring the rod's bore 1024. The second one of the open ends of the opening 1020 can include a second enlarged diameter portion 1020e, as in this illustrated embodiment, which can facilitate movement of the first connector 1002 relative to the second connector 1006, as discussed further below. A reduced diameter portion 1020r of the opening 1020 can extending between the first and second enlarged diameter portions 1020d, 1020e.

The first connector 1002 can be configured to be longitudinally movable relative to the second connector 1006 within the opening 1020. The first connector 1002, e.g., the washer 1010 thereof, can be configured to be advanced into the opening 1020 through the second one of the opening's open ends. In this illustrated embodiment, the first connector 1002 is configured to be manually coupled to the second connector 1002 by being advanced into the opening 1020. In an exemplary embodiment, the first connector 1002 can be inserted into the opening 1020 before the rod 1018 is coupled to the second connector 1006 in order to help provide adequate space for insertion of the first connector 1002 into the opening 1020. In other embodiments, the first connector 1002 can be pre-loaded into the opening 1020 and/or can be non-removable therefrom.

The washer 1010 can be configured to move within the opening 1020 in a channel 1032 defined by the opening's bottom surface 1030 and an interior surface 1034s of a ledge 1034 overhanging either side of the opening 1020. The first connector 1002 can thus be configured to be longitudinally movable relative to the second connector 1006 by sliding in the channel 1032 defined therein.

The first connector 1002 can include a guide portion 1028 configured to facilitate the movement of the first connector 1002 relative to the second connector 1006. The guide portion 1028 can be configured to cooperate with the bottom surface 1030 of the opening 1020 to guide the first connector 1002 within the opening 1020. The guide portion 1028 can be configured to stop longitudinal movement of the first connector 1002 in a first direction R1 relative to the second connector 1006, which can help retain the first connector 1002 within the opening 1020 and/or help prevent the first connector 1002 from moving too far and damaging tissue and/or other material adjacent a target bone.

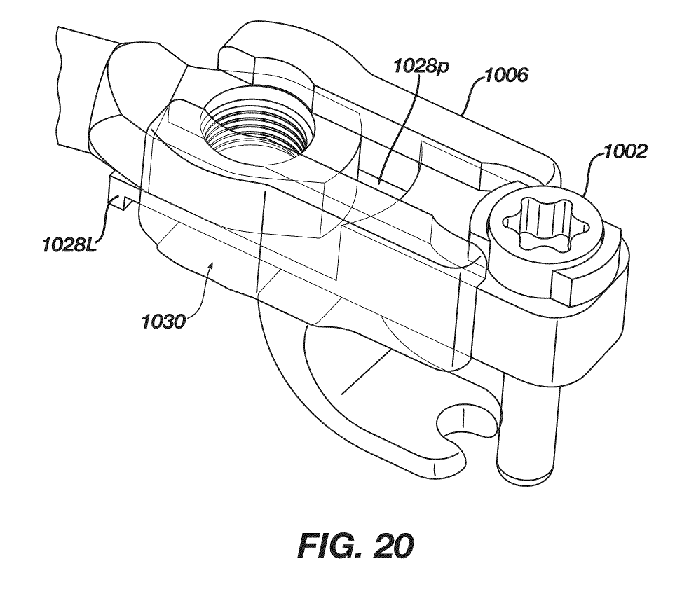

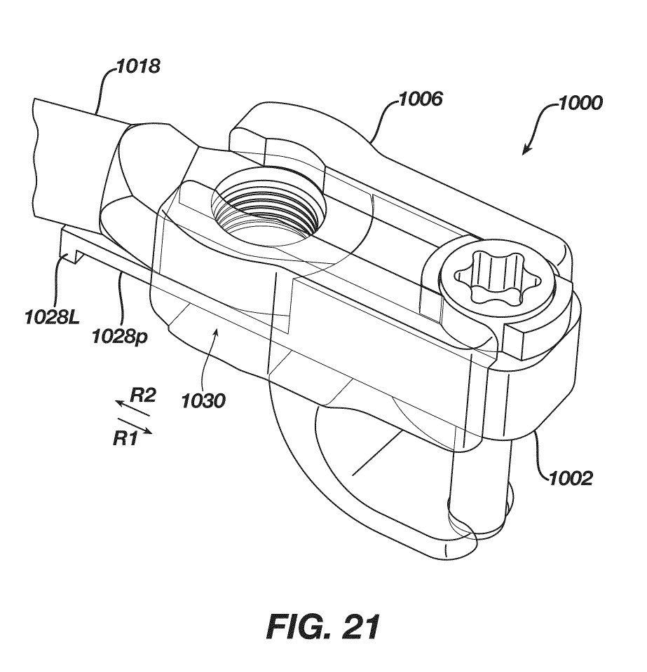

As in this illustrated embodiment, the guide portion 1028 can include an elongate plate 1028p having a lip 1028L extending therefrom. The elongate plate 1028p can extend from the washer 1010 in a direction away from the elongate post 1004, e.g., in a second direction R2 that is opposite the first direction R1. The elongate plate 1028p can be configured to slide along the bottom surface 1030 of the second connector's opening 1020. The lip 1028L can be configured to abut the bottom surface 1030 when the first connector 1002 is in a first terminal position in which the first connector 1002 is advanced as far as possible in the first direction R1, as shown in FIGS. 17-19. The lip 1028L can be configured to not abut and be spaced a distance apart from the bottom surface 1030 when the first connector 1002 has been moved in the second direction R2 from the first terminal position. FIG. 20 shows the first connector 1002 moved in the second direction R2 from the first terminal position to an intermediate position in which the lip 1028L is at a first distance from the bottom surface 1030. FIG. 21 shows the first connector 1002 moved in the second direction R2 from the intermediate position of FIG. 20 to a second terminal position in which the lip 1028L is at a second distance from the bottom surface 1030 that is greater than the first distance and in which the first connector 1002 is advanced as far as possible in the second direction R2. The intermediate position illustrated in FIG. 20 is only one of a plurality of possible intermediate positions possible for the first connector 1002 between the first and second terminal positions. The second enlarged diameter portion 1020e of the opening 1020 can be configured to seat the head 1012 of the first connector 1002 therein, as shown in FIG. 21. The reduced diameter portion 1020r of the opening 1020 can prevent the head 1012 from moving any farther in the second direction R2. In other words, the opening 1020 can be configured to cooperate with the first connector 1002, e.g., the head 1012 thereof, to stop longitudinal movement of the first connector 1002 in the second direction R2 relative to the second connector 1006, which can help prevent the first connector 1002 from moving too far and damaging tissue and/or other material adjacent a target bone.

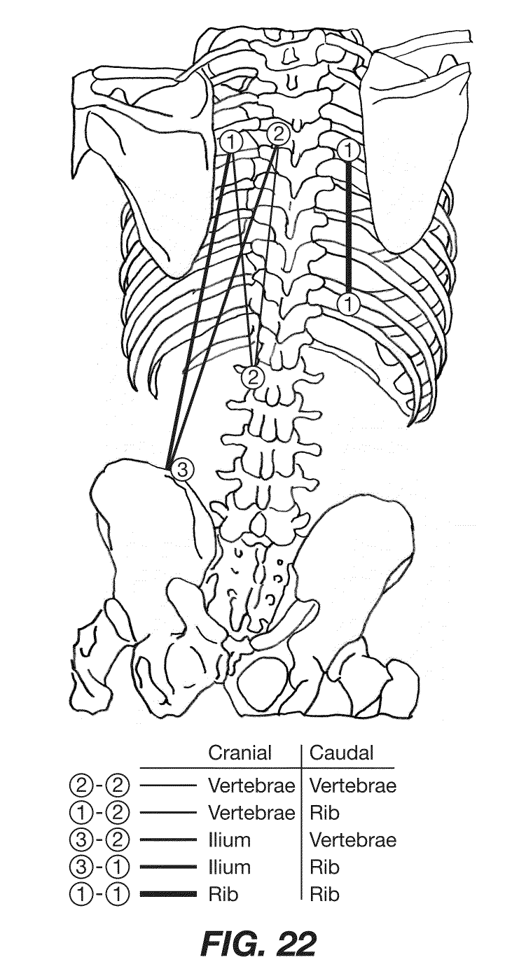

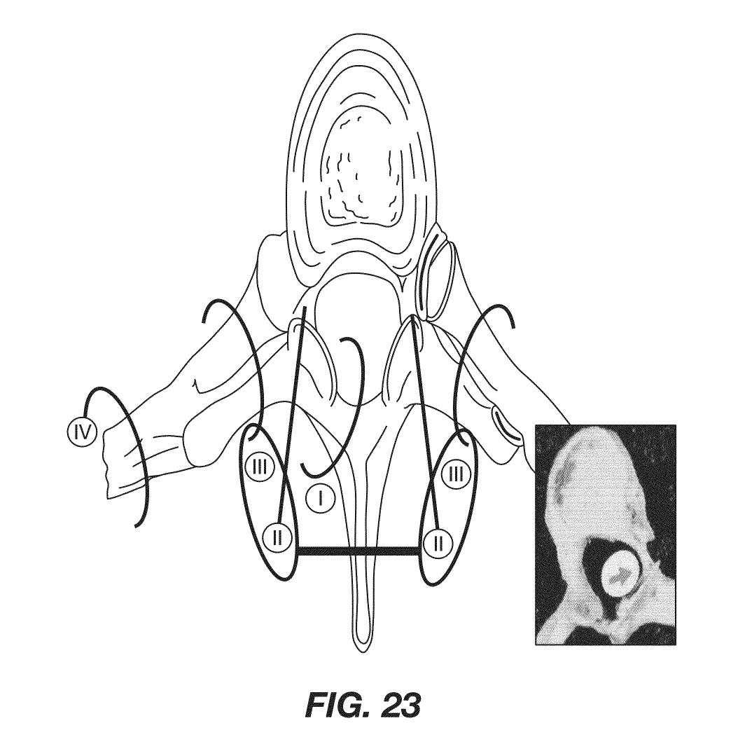

The embodiments in FIGS. 1-21 of surgical devices configured for anchoring spinal rods can each be configured to attach to a patient's rib via cooperating rib seats of the device, and can each be configured to couple to the rod. FIG. 22 illustrates examples of locations, identified by the reference \textcircled{1}, where a first device can be attached to a rib, e.g., where the rib seats of the first device can seat the rib. FIG. 23 illustrates another example location, identified by reference line IV, where the first device can be attached to a rib, e.g., where the rib seats of the first device can seat the rib. As also mentioned above, the rod attached to the first device can extend to a second device attached to another bone of the patient, such as a rib, pelvic bone, or vertebra. FIG. 22 illustrates an example of a location where a second device can be attached to a second rib with a rod 10 extending between the first and second devices, an example of a location where a second device can be attached to an ilium, identified by the reference \textcircled{3}, with a rod 12 extending between the first and second devices, and an example of a location where a second device can be attached to a vertebra, identified by the reference \textcircled{2}, with a rod 14 extending between the first and second devices. FIG. 23 illustrates examples of locations where a surgical device can be attached to a vertebra, e.g., a lamina (identified by reference line I), a pedicle (identified by reference line II), and a transverse process (identified by reference line III). In some embodiments, a spinal rod can extend between first and second devices each attached to a bone other than a rib. FIG. 22 illustrates an example of a location where a first device can be attached to a vertebra and a second device can be attached to another vertebra with a rod 16 extending between the first and second devices, and FIG. 22 illustrates an example of a location where a first device can be attached to a vertebra and a second device can be attached to the ilium with a rod 18 extending between the first and second devices.

As mentioned above, a surgical device configured to attach to a bone of a patient to facilitate anchoring of an elongate rod coupled thereto can be configured to allow cross connection. In general, the device can be configured to attach to a bone to facilitate anchoring of a first elongate rod (e.g., a spinal rod) and a second elongate rod (e.g., a cross connection rod) thereto. The first elongate rod can be configured to couple to a second surgical device attached to a second bone, e.g., using a surgical device discussed herein, so as to extend between the first and second surgical devices. The second elongate rod can be configured to couple to a third surgical device attached to a third bone, e.g., using a surgical device discussed herein, so as to extend between the first and third surgical devices. As will be appreciated by a person skilled in the art, cross connection can help provide stability to the system including the first, second, and third devices and the first and second elongate rods, which can facilitate patient treatment.

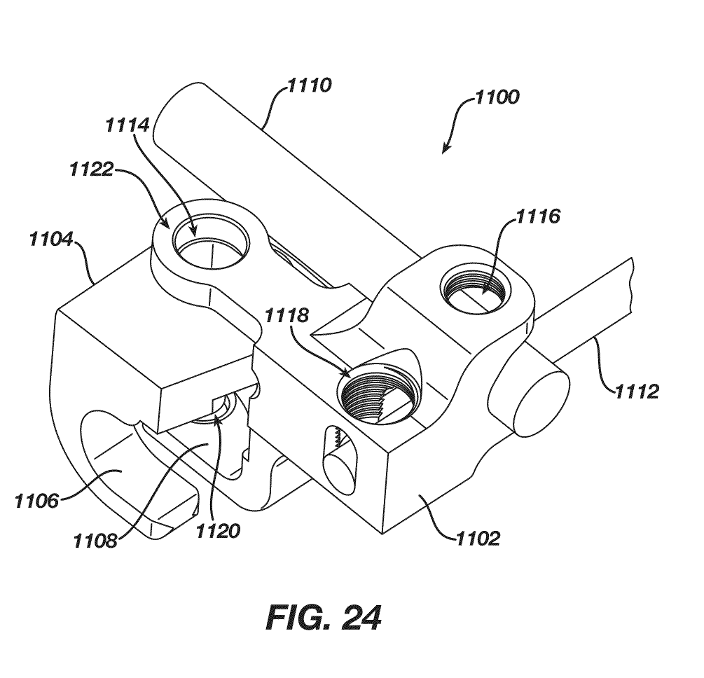

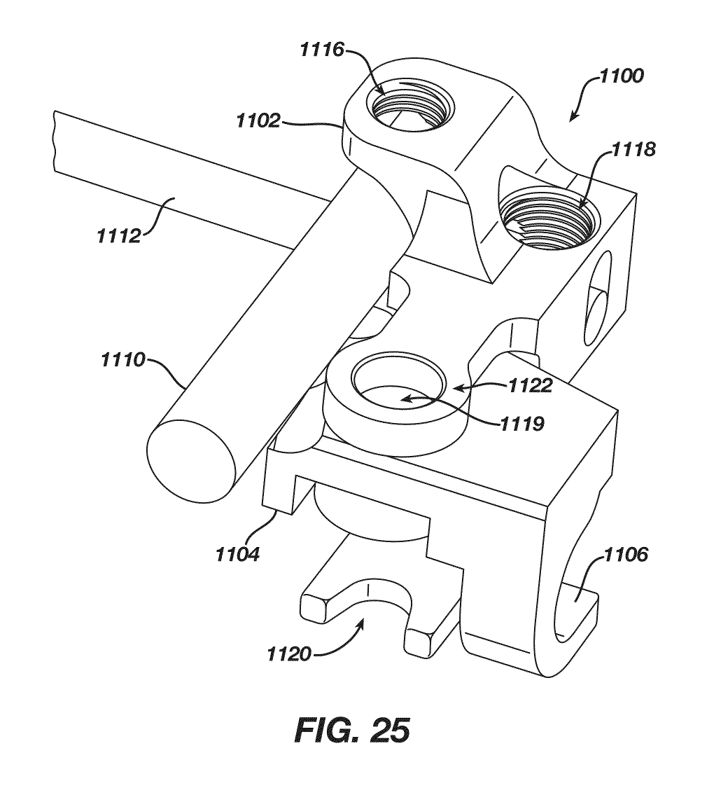

FIGS. 24 and 25 illustrate one embodiment of a surgical device 1100 configured to facilitate spinal rod anchoring and to facilitate cross connection. The device 1100 can be configured to allow cross connection to a thoracic vertebra of a patient, going around the patient's transverse process. As in this illustrated embodiment, the device 1100 can include a first connector 1102 and a second connector 1104 configured to couple to the first connector 1102. The first connector 1102 can include a first seat 1108 configured to seat a bone (e.g., a rib) therein. The second connector 1104 can include a second seat 1106 configured to seat the bone therein. The first connector 1102 can be configured to couple to a first elongate rod 1110 (e.g., a spinal rod), and can be configured to couple to a second elongate rod 1112 (e.g., a cross connector rod). The first connector 1102 can include a first bore 1114 configured to receive a first locking member therein to facilitate secure attachment of the first and second connectors 1102, 1104 together in a fixed position relative to one another, a second bore 1116 configured to receive a second locking member therein to engage the first rod 1110 to facilitate secure attachment of the first rod 1110 and the first connector 1102 in a fixed position relative to one another, and a third bore 1118 configured to receive a third locking member therein to engage the second rod 1112 to facilitate secure attachment of the second rod 1112 and the first connector 1102 in a fixed position relative to one another. The second connector 1104 can include a groove 1120 formed therein and configured to cooperate with the first bore 1114 to facilitate secure attachment of the first and second connectors 1102, 1104 together in a fixed position relative to one another. As in this illustrated embodiment, the groove 1120 can be formed in an end of the second seat 1108. The groove 1120 can be aligned with the first bore 1114 such that when the first locking member extends through the first bore 1114, and optionally has a head seated on a surface 1122 of the first connector 1102 that surrounds the first bore 1114, the first locking member can be seated in the groove 1120, which can help keep the first locking member in position relative to the first and second connectors 1102, 1104.

In some embodiments of surgical devices configured to facilitate spinal rod anchoring and to facilitate cross connection, the surgical device can include a receiver member configured to receive the spinal rod therein. The receiver member can be a discrete element configured to be independently manipulated from a bone connector of the surgical device (e.g., first and second connectors of the device) that the receiver member can be configured to be attached to, which can facilitate manipulation of the spinal rod. The receiver member can include a rod seat configured to seat the spinal rod therein. The rod seat can be side-loading or top-loading. The rod seat can be configured as a monoaxial rod seat that limits the rod seated therein to monoaxial movement relative to the bone connector. Alternatively, the rod seat can be configured as a polyaxial rod seat that allows the rod seated therein to move polyaxially relative to the receiver member.

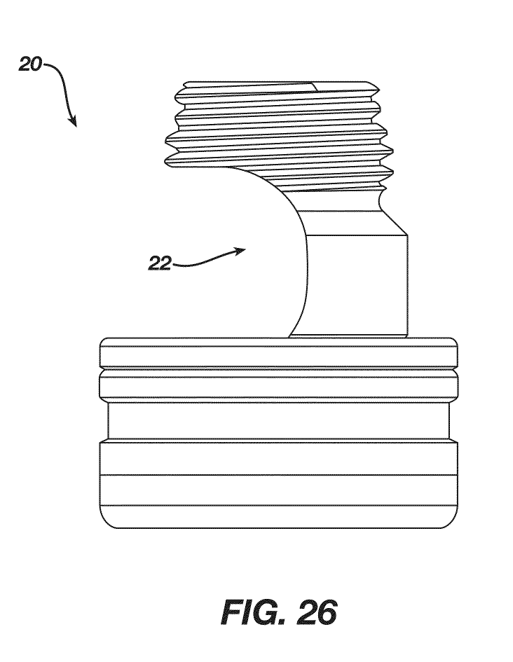

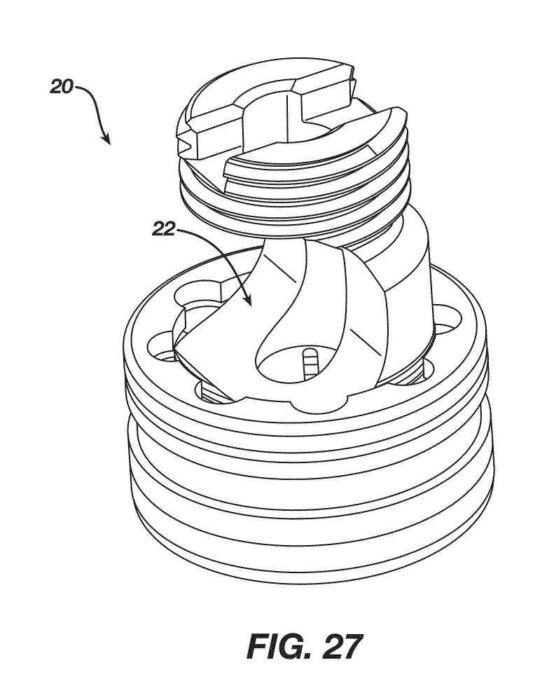

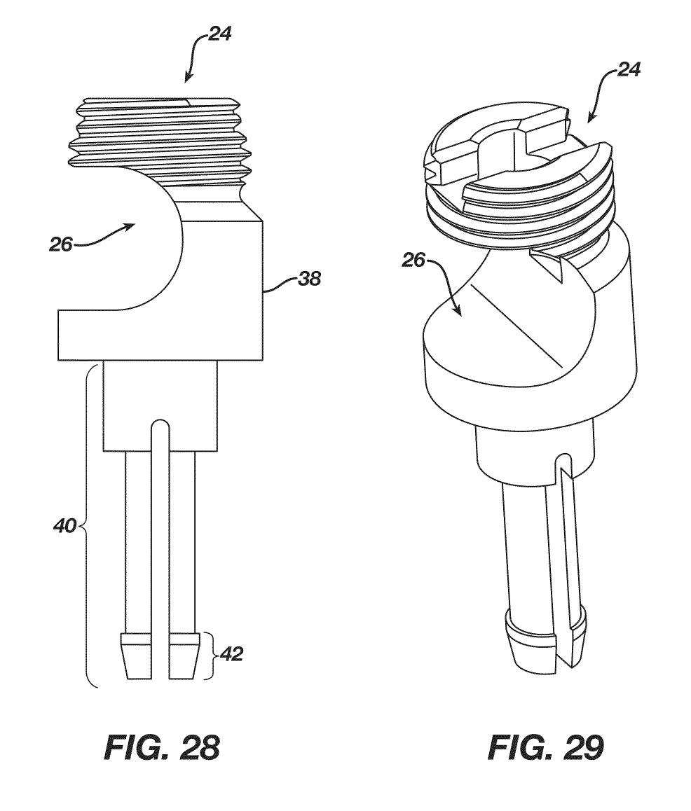

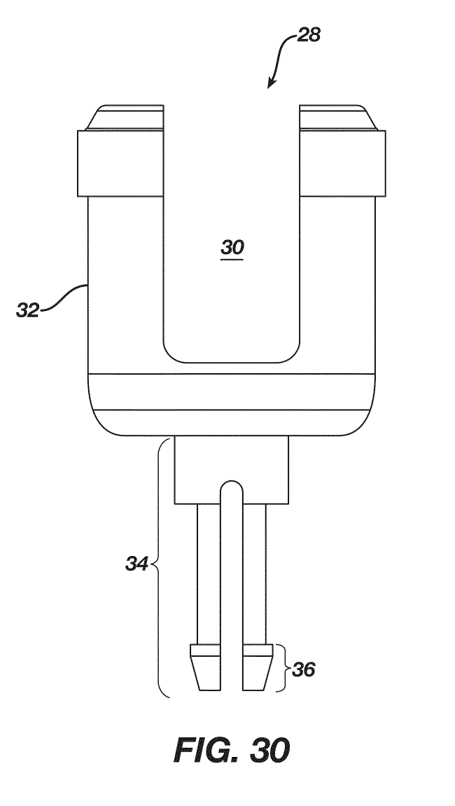

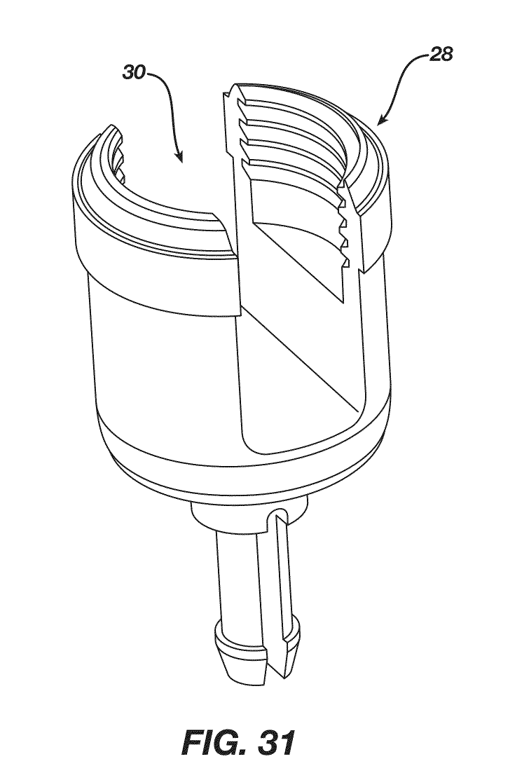

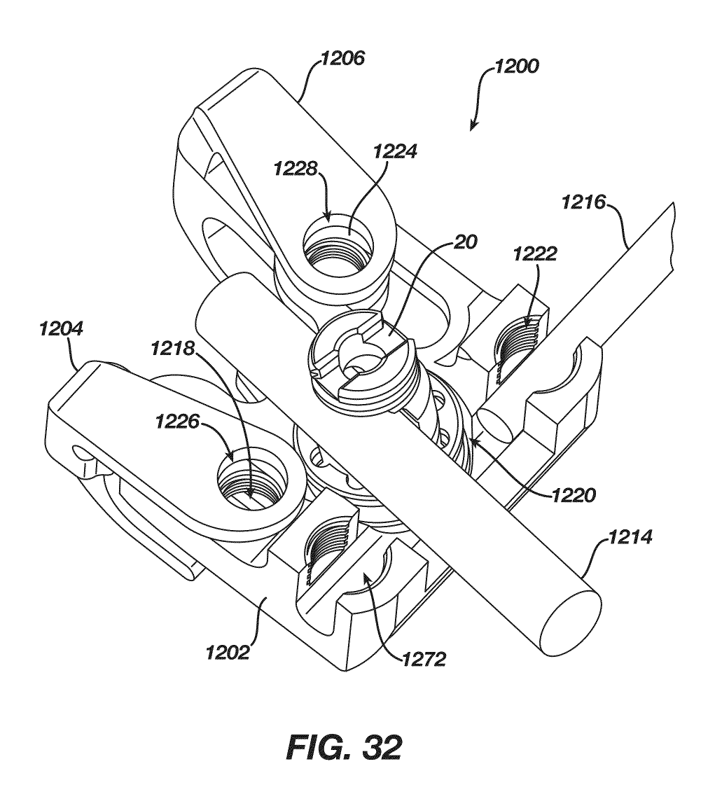

FIGS. 26 and 27 illustrate one embodiment of a receiver member 20 including a rod seat 22. The rod seat 22 in this illustrated embodiment is side-loading and is configured to allow polyaxial movement of a rod (not shown) seated therein relative to a bone connector (not shown) to which the receiver member 20 is coupled. FIGS. 28 and 29 illustrate another embodiment of a receiver member 24 including a rod seat 26. The rod seat 26 in this illustrated embodiment is side-loading and is configured to limit a rod (not shown) seated therein to monoaxial movement relative to a bone connector (not shown) to which the receiver member 24 is coupled. FIGS. 30 and 31 illustrate yet another embodiment of a receiver member 28 including a rod seat 30. The rod seat 30 in this illustrated embodiment is top-loading and is configured to limit a rod (not shown) seated therein to monoaxial movement relative to a bone connector (not shown) to which the receiver member 28 is coupled.

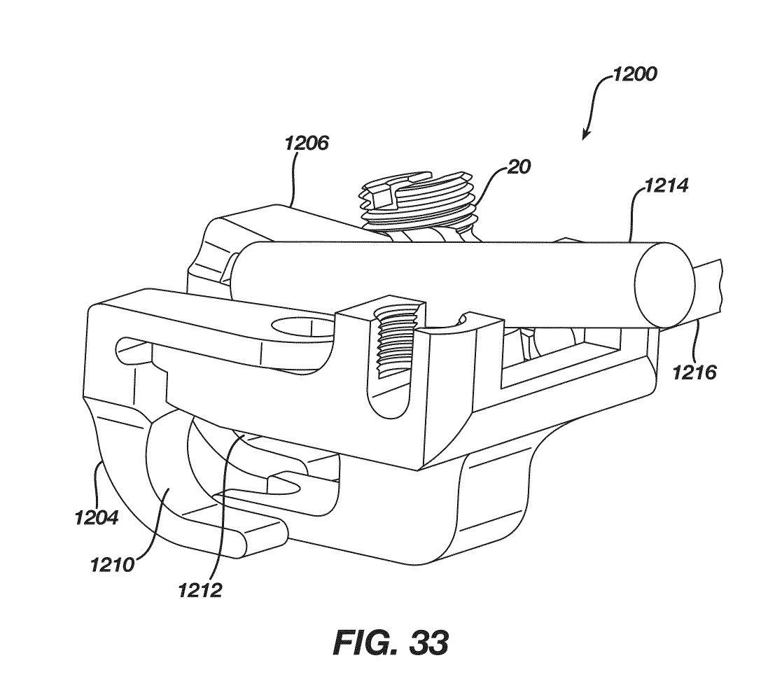

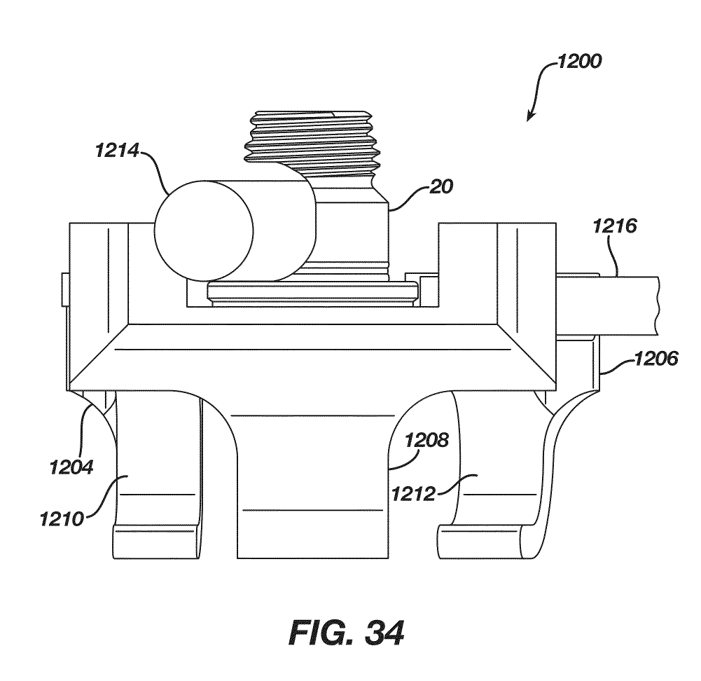

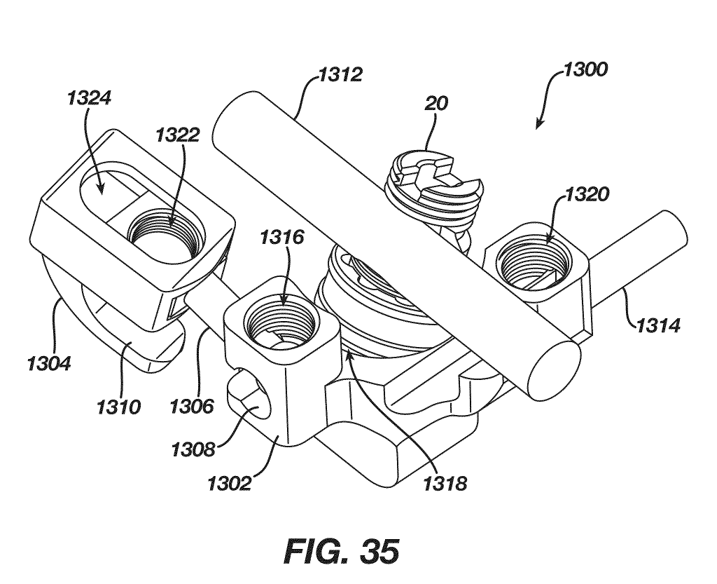

FIGS. 32-34 illustrate one embodiment of a surgical device 1200 configured to facilitate spinal rod anchoring, configured to facilitate cross connection, and configured to couple to the side-loading polyaxial receiver member 20 of FIG. 26. The device 1200 can be configured to allow cross connection to a thoracic vertebra of a patient, going around the patient's transverse process. As in this illustrated embodiment, the device 1200 can include a first connector 1202, a second connector 1204 configured to couple to the first connector 1202, and a third connector 1206 configured to couple to the first connector 1202. The first connector 1202 can include a first seat 1208 configured to seat a bone (e.g., a pedicle) therein. The second connector 1204 can include a second seat 1210 configured to seat the bone therein, and the third connector 1206 can include a third seat 1212 configured to seat the bone therein. The second and third connectors 1204, 1206 can be discrete elements from each other. The second and third seats 1210, 1212 can generally cooperate together to perform similar to the second and third seats 112, 114 of the second connector 104 of FIG. 1. The device 1200 can thus be configured to have three points of contact with a bone seated by the seats 1208, 1210, 1212.

The first connector 1202 can be configured to couple to a first elongate rod 1214 (e.g., a spinal rod), and can be configured to couple to a second elongate rod 1216 (e.g., a cross connector rod). The first connector 1202 can include a first bore 1218 configured to receive a first locking member therein to facilitate secure attachment of the first and second connectors 1202, 1204 together in a fixed position relative to one another, a second bore 1220 configured to receive the receiver member 20 therein to engage the first rod 1214 to facilitate secure attachment of the first rod 1214 and the first connector 1202 in a fixed position relative to one another, a third bore 1222 configured to receive a third locking member therein to engage the second rod 1216 to facilitate secure attachment of the second rod 1216 and the first connector 1202 in a fixed position relative to one another, and a fourth bore 1224 configured to receive a fourth locking member therein to facilitate secure attachment of the first and third connectors 1202, 1206 together in a fixed position relative to one another. The receiver member 20, and hence also the first rod 1214 seated therein, can be configured to be polyaxially movable within the second bore 1220 relative to the first connector 1202. The first connector 1202 can, as in this illustrated embodiment, include a second third bore 1222, which can allow cross connection via the second rod 1216 in a selected one of two directions and/or allow the second rod 1216 to be secured to the first connector 1202 using two locking members, one in each of the third bores 1222. The second connector 1204 can include a bore 1226 formed therethrough and configured to align with the first bore 1218 and be configured to also receive the first locking member therein to facilitate secure attachment of the first and second connectors 1202, 1204 together in a fixed position relative to one another. The third connector 1206 can include a bore 12268 formed therethrough and configured to align with the fourth bore 1224 and be configured to also receive the fourth locking member therein to facilitate secure attachment of the first and second connectors 1202, 1204 together in a fixed position relative to one another.

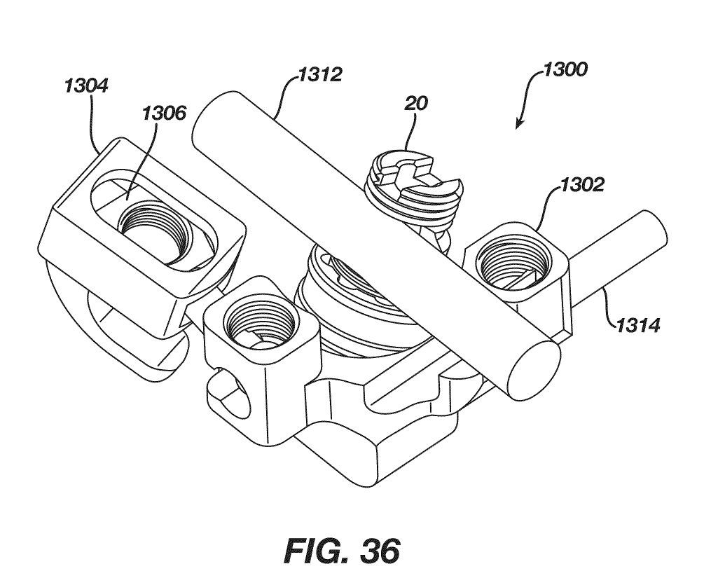

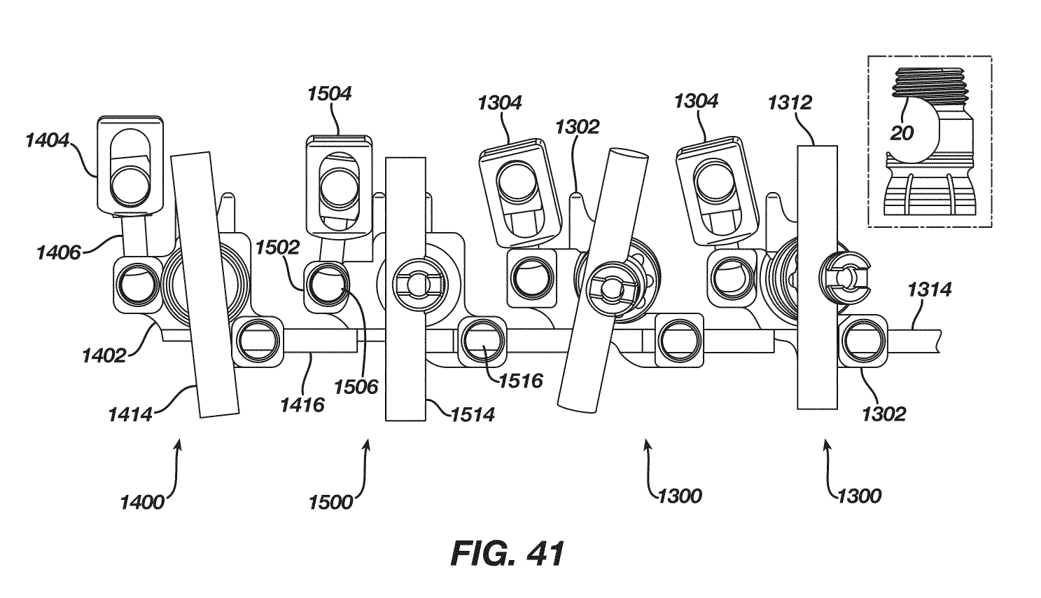

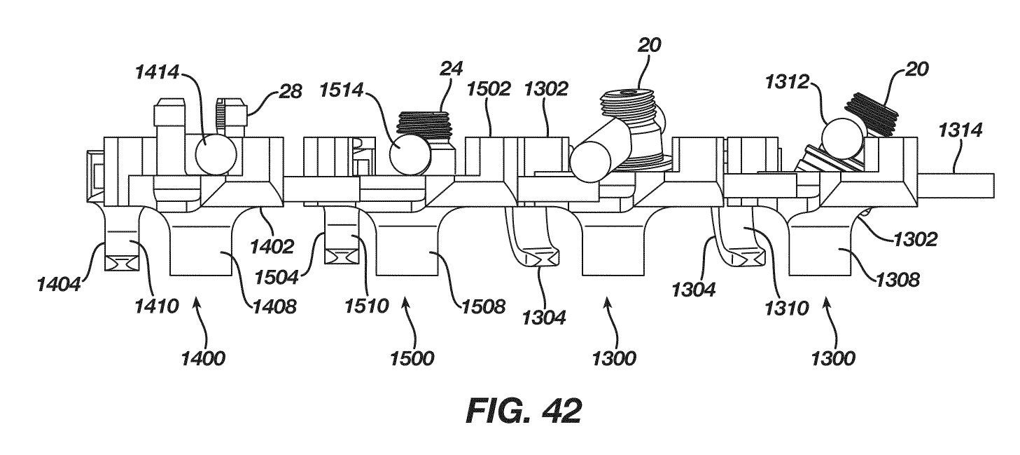

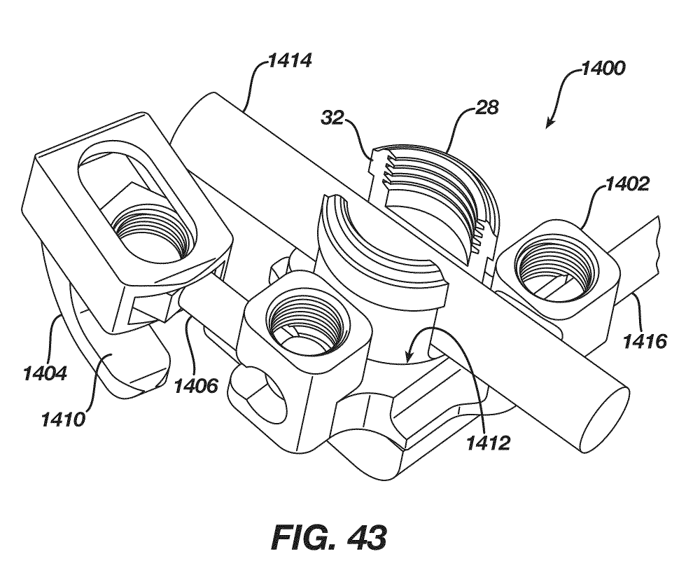

FIGS. 35-39 illustrate another embodiment of a surgical device 1300 configured to facilitate spinal rod anchoring, configured to facilitate cross connection, and configured to couple to the side-loading polyaxial receiver member 20 of FIG. 26. The device 1300 can be configured to allow cross connection to a thoracic vertebra of a patient, going around the patient's transverse process. FIGS. 40-42 also illustrate the device 1300, along with various other embodiments of surgical devices, discussed below. The surgical devices illustrated in FIGS. 40-42 would not necessarily be implanted within a patient in this configuration with respect to one another. The configuration shown in FIGS. 40-42 is merely an illustrative example. FIG. 42 shows that the polyaxial receiver member 28 embodiment can have a taller profile than the monoaxial receiver member embodiments 20, 24, which can make the monoaxial receiver member 20, 24 embodiments more desirable to use in situations with particularly limited space within a patient's body. FIG. 41 also includes an insert illustrating the side-loading polyaxial receiver member 20 of FIG. 26.

As in this illustrated embodiment, the device 1300 can include a first connector 1302, a second connector 1304 configured to couple to the first connector 1302, and a third connector 1306 configured to couple to and extend between the first and second connectors 1302, 1304. The first connector 1302 can include a first seat 1308 configured to seat a bone (e.g., a pedicle) therein. The second connector 1304 can include a second seat 1310 configured to seat the bone therein. The third connector 1306 can be configured to allow polyaxial adjustment of the second connector 1304 relative to the first connector 1302.

The first connector 1302 can be configured to couple to a first elongate rod 1312 (e.g., a spinal rod), and can be configured to couple to a second elongate rod 1314 (e.g., a cross connector rod). The first connector 1302 can include a first bore 1316 configured to receive a first locking member therein to facilitate secure attachment of the first and third connectors 1302, 1306 together in a fixed position relative to one another, a second bore 1318 configured to receive the receiver member 20 therein to engage the first rod 1312 to facilitate secure attachment of the first rod 1312 and the first connector 1302 in a fixed position relative to one another, and a third bore 1320 configured to receive a third locking member therein to engage the second rod 1314 to facilitate secure attachment of the second rod 1314 and the first connector 1302 in a fixed position relative to one another. The third connector 1306 can include a fourth bore 1322 configured to receive a fourth locking member therein to facilitate secure attachment of the first and second connectors 1302, 1304 together in a fixed position relative to one another. The first and fourth bores 1316, 1322 can be configured to cooperate together, along with the first and fourth locking members configured to be respectively received therein, to lock the first and second connectors 1302, 1304 in a fixed position relative to one another. The receiver member 20, and hence also the first rod 1312 seated therein, can be configured to be polyaxially movable within the second bore 1318 relative to the first connector 1302. The second connector 1304 can include an opening 1324 formed therethrough and configured to align with the fourth bore 1322 and be configured to also receive the fourth locking member therein to facilitate secure attachment of the first and second connectors 1302, 1304 together in a fixed position relative to one another. As in the illustrated embodiment, the opening 1324 can have an elongate shape. The elongate shape can allow the third connector 1306 to be positioned any of a plurality of positions relative to second connector 1304 while allowing the fourth bore 1322 to be aligned with the opening 1324, which can facilitate desired positioning of the first and second connectors 1302, 1304 relative to a bone seated thereby.

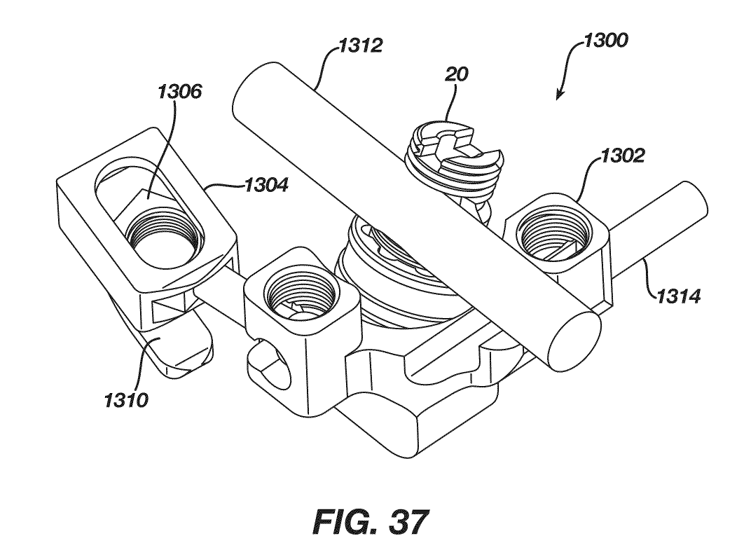

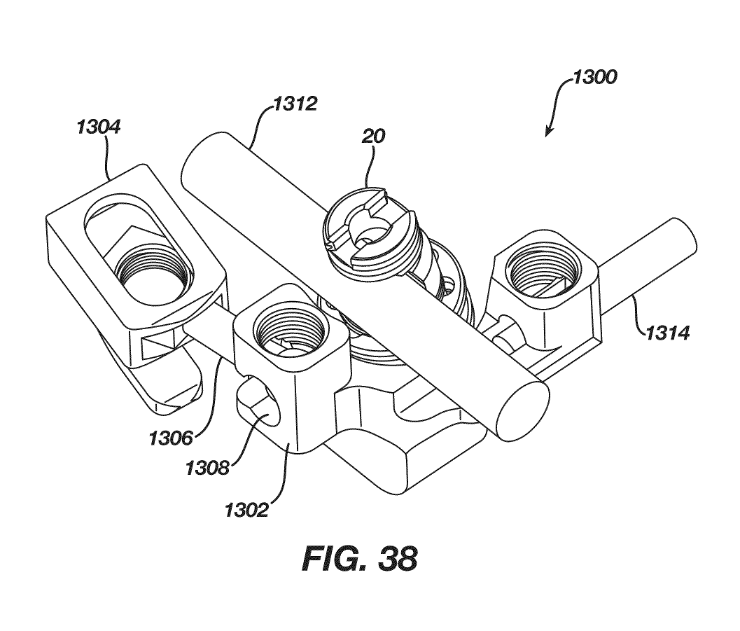

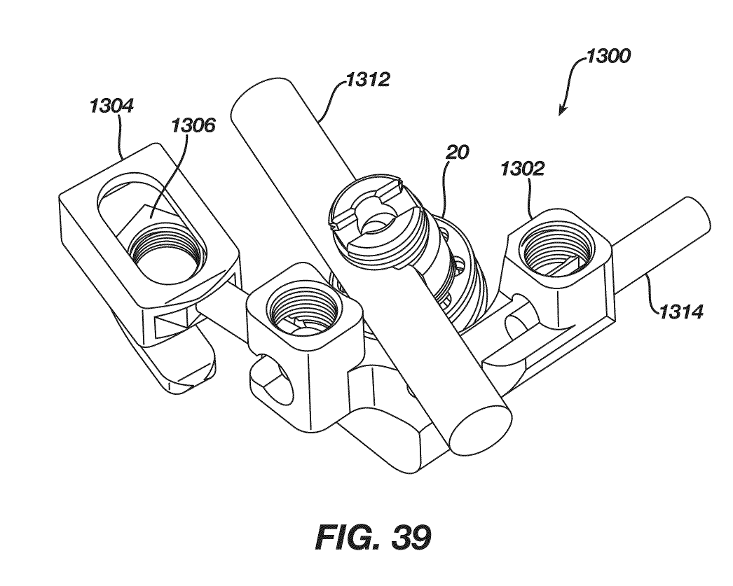

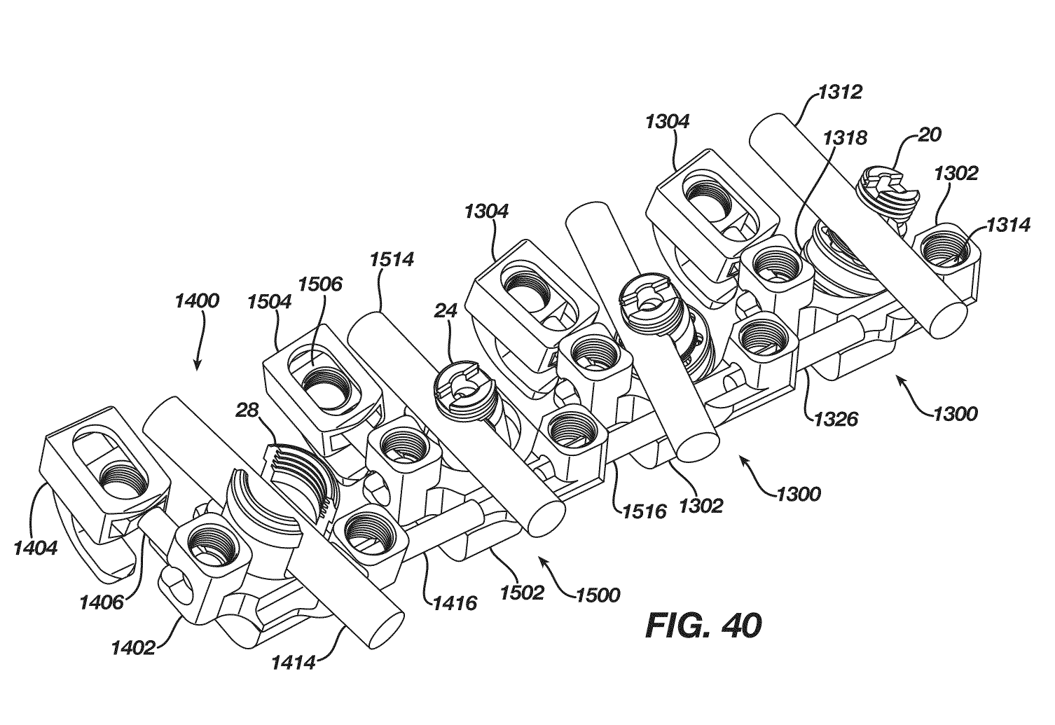

FIG. 36 illustrates the second connector 1304 adjusted in position from FIG. 35 relative to the third connector 1306 and the first connector 1302. FIG. 37 illustrates the second connector 1304 adjusted in position from FIG. 36 relative to the third connector 1306 and the first connector 1302. FIG. 38 illustrates the receiver member 20, and hence the first rod 1312 coupled thereto, adjusted in position from FIG. 37 relative to the first, second, and third connectors 1302, 1304, 1306. FIG. 39 illustrates the receiver member 20, and hence the first rod 1312 coupled thereto, adjusted in position from FIG. 38 relative to the first, second, and third connectors 1302, 1304, 1306.

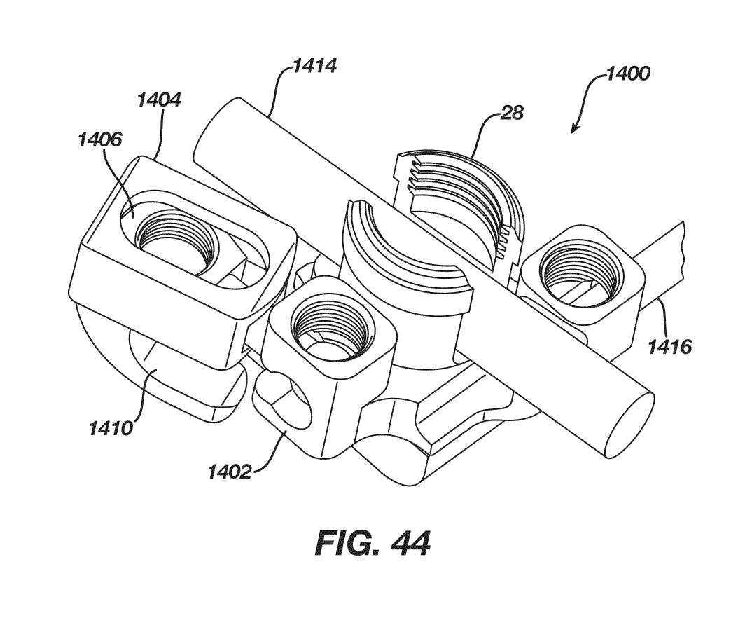

FIGS. 43-45A illustrate another embodiment of a surgical device 1400 configured to facilitate spinal rod anchoring, configured to facilitate cross connection, and configured to couple to the top-loading monoaxial receiver member 28 of FIG. 30. The device 1400 can be configured to allow cross connection to a thoracic vertebra of a patient, going around the patient's transverse process. FIGS. 40-42 also illustrate the device 1400.

As in this illustrated embodiment, the device 1400 can include a first connector 1402, a second connector 1404 configured to couple to the first connector 1402, and a third connector 1406 configured to couple to and extend between the first and second connectors 1402, 1404. The first connector 1402 can include a first seat 1408 configured to seat a bone (e.g., a pedicle) therein. The second connector 1404 can include a second seat 1410 configured to seat the bone therein. The third connector 1406 can be configured to allow polyaxial adjustment of the second connector 1404 relative to the first connector 1402. The first, second, and third connectors 1402, 1404, 1406 can generally be configured and used similar to the first, second, and third connectors 1302, 1304, 1306 of FIG. 35. Unlike in the embodiment of FIG. 35, in the embodiment of FIG. 43, the first connector 1402 can be configured to couple to the top-loading monoaxial receiver member 28, e.g., include a second bore 1412 configured to receive the receiver member 20 therein to engage a first elongate rod 1414 (e.g., a spinal rod) to facilitate secure attachment of the first rod 1414 and the first connector 1402 in a fixed position relative to one another. FIGS. 43-46 also illustrate the first connector 1402 coupled to a second elongate rod 1416 (e.g., a cross connector rod).

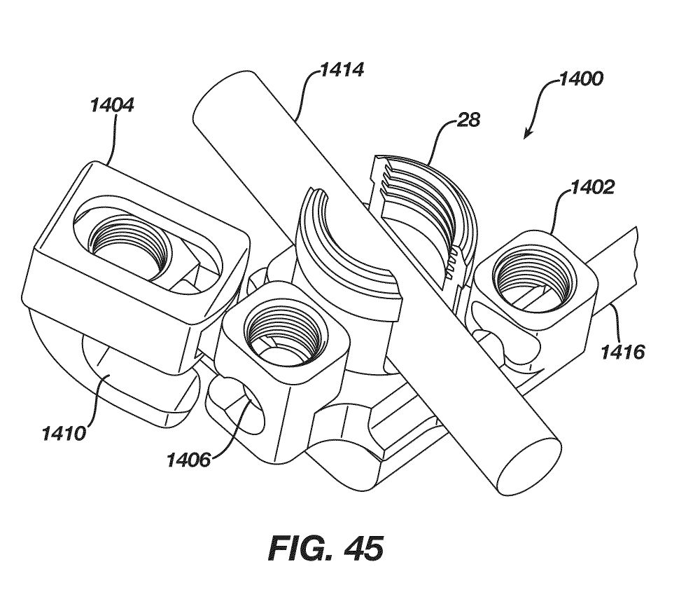

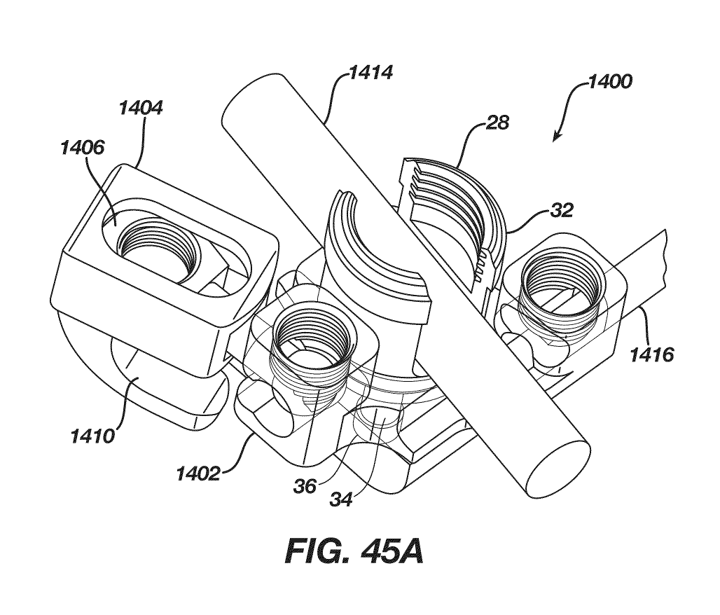

FIG. 44 illustrates the second connector 1404 adjusted in position from FIG. 43 relative to the third connector 1406 and the first connector 1402. FIGS. 45 and 45A illustrate the receiver member 28, and hence the first rod 1412 coupled thereto, adjusted in position from FIG. 44 relative to the first, second, and third connectors 1402, 1404, 1406.

As illustrated in FIGS. 30 and 45A, the receiver member 28 can include a head 32 with an elongate shaft 34 extending distally therefrom. The elongate shaft 34 can include a distal tip 36 configured to lock the receiver member 28 within the second bore 1412, e.g., by snap-fitting therein. The receiver member 28 can be non-removable from the second bore 1412 or can be releasable therefrom. In some embodiments, the receiver member 28 can be pre-loaded into the second bore 1412. In other embodiments, the receiver member 28 can be insertable into the second bore 1412 by a user, which can allow the user to select an appropriate receiver member, e.g., based on size of the receiver member's rod seat.

FIGS. 40-42 illustrate another embodiment of a surgical device 1500 configured to facilitate spinal rod anchoring, configured to facilitate cross connection, and configured to couple to the side-loading monoaxial receiver member 24 of FIG. 28. The device 1500 can be configured to allow cross connection to a thoracic vertebra of a patient, going around the patient's transverse process.

As in this illustrated embodiment, the device 1500 can include a first connector 1502, a second connector 1504 configured to couple to the first connector 1502, and a third connector 1506 configured to couple to and extend between the first and second connectors 1502, 1504. The first connector 1502 can include a first seat 1508 configured to seat a bone (e.g., a pedicle) therein. The second connector 1504 can include a second seat 1510 configured to seat the bone therein. The third connector 1506 can be configured to allow polyaxial adjustment of the second connector 1504 relative to the first connector 1502. The first, second, and third connectors 1502, 1504, 1506 can generally be configured and used similar to the first, second, and third connectors 1302, 1304, 1306 of FIG. 35. Unlike in the embodiment of FIG. 35, in the embodiment of FIG. 40, the first connector 1502 can be configured to couple to the side-loading monoaxial receiver member 24, e.g., include a second bore 1512 configured to receive the receiver member 24 therein to engage a first elongate rod 1514 (e.g., a spinal rod) to facilitate secure attachment of the first rod 1514 and the first connector 1502 in a fixed position relative to one another. FIGS. 40-42 also illustrate the first connector 1502 coupled to a second elongate rod 1516 (e.g., a cross connector rod). Similar to the receiver member 28 of FIG. 30, the receiver member 24 of FIG. 28 can include a head 38, an elongate shaft 40 extending distally from the head 38, and a distal tip 42 of the shaft 40 configured to lock the receiver member 24 within a second bore (not shown) of the first connector 1502, e.g., by snap-fitting therein.

FIGS. 40-42 illustrate two of the devices 1300 of FIG. 35. In the device 1300 on the left in FIGS. 40-42, the first connector 1302 is seating two second elongate rods, e.g., the second elongate rod 1516 seated by the device 1500 and another second elongate rod 1326. In the device 1300 on the right in FIGS. 40-42, the first connector 1302 is seating the second rods 1314, 1326.

In some embodiments of surgical devices configured to facilitate spinal rod anchoring and to facilitate cross connection, the surgical device can include a single bone connector configured to couple to a first elongate rod (e.g., a spinal rod) and to a second elongate rod (e.g., a cross connector rod). The device can be configured to movably seat a rod seating member therein. The rod seating member can be configured to seat the first elongate rod and be configured to facilitate adjustment of the first elongate rod relative to the bone connector. In some embodiments, the rod seating member can be non-removably coupled to the bone connector, e.g., pre-loaded therein. In other embodiments, the rod seating member can be removably coupled to the bone connector, e.g., selectively insertable into and releasable from the bone connector.

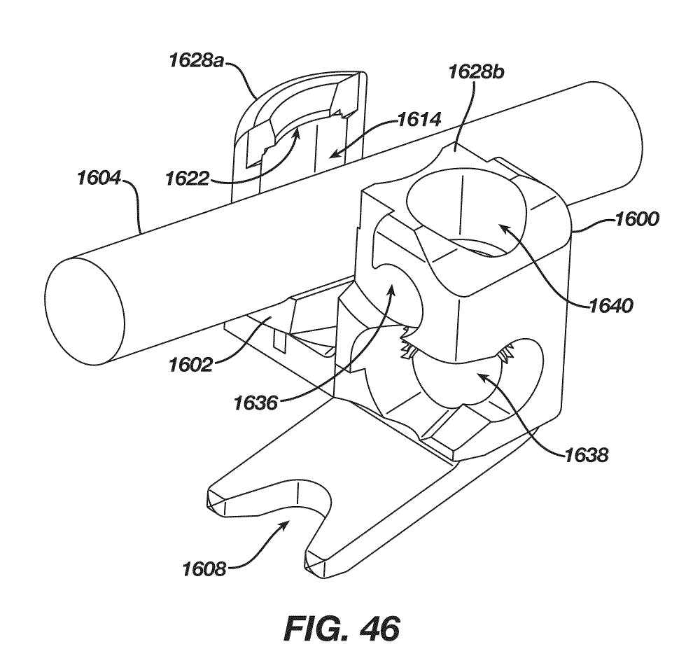

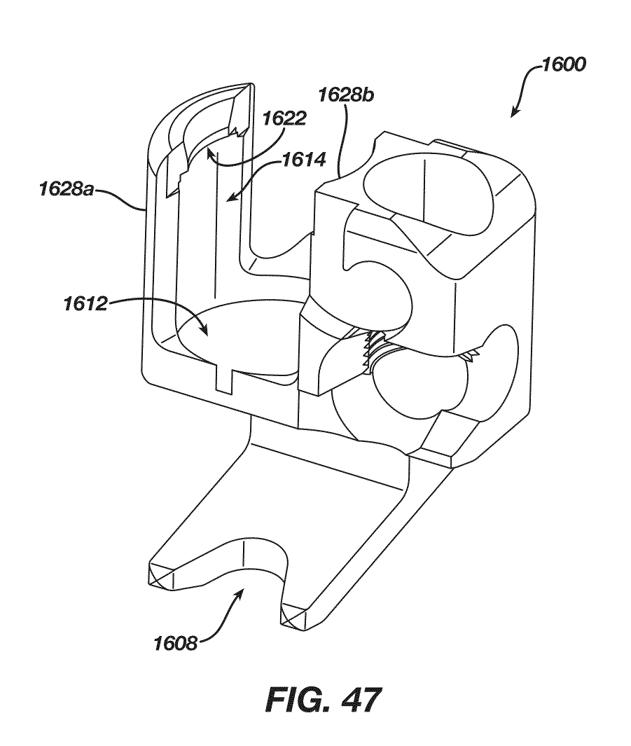

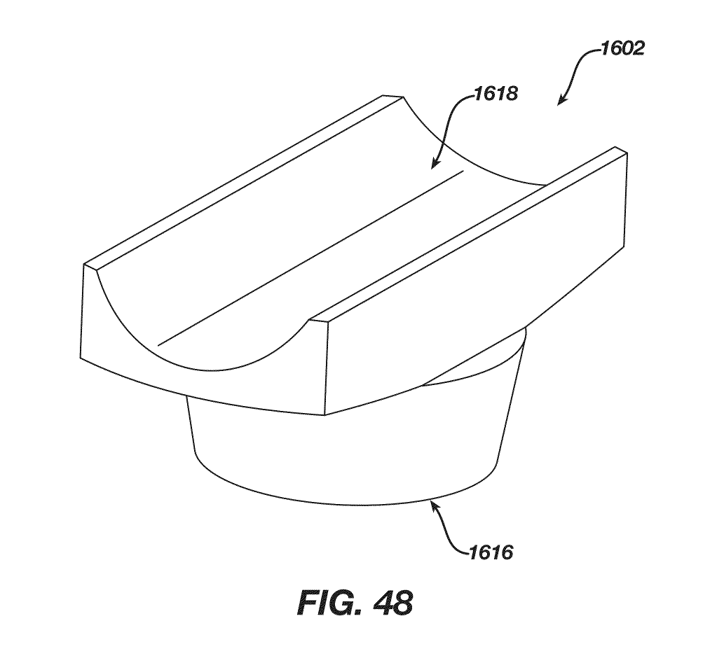

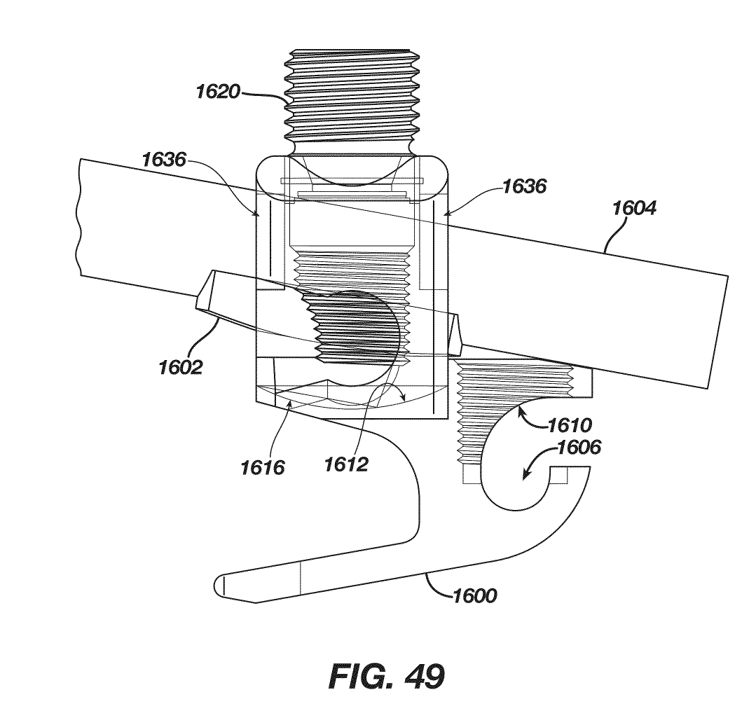

FIGS. 46 and 47 illustrate one embodiment of a surgical device 1600 including a single bone connector configured to facilitate spinal rod anchoring and to facilitate cross connection. FIGS. 49-54 also illustrate the device 1600. The device 1600 can be configured to allow cross connection to a thoracic vertebra of a patient, going around the patient's transverse process. The device 1600 can be configured to couple to a rod seating member 1602, which is also illustrated in FIGS. 48-53. The rod seating member 1602 can be configured to seat a first elongate rod 1604 (e.g., a spinal rod) and can be configured to move relative to the bone connector 1600, thereby allowing adjustment of a position of the first rod 1604 relative to the bone connector 1600, as discussed further below. In this illustrated embodiment, the rod seating member 1602 is removably coupled to the bone connector 1600.

The device 1600 can include a cross connector rod seat 1606 (obscured in FIG. 46 but illustrated in FIGS. 49-53) configured to seat a second elongate rod (not shown), e.g., cross connector rod, therein. The device 1600 can include a bore 1610 configured to receive a locking member therein to engage the second rod to facilitate secure attachment of the second rod and the bone connector 1600 in a fixed position relative to one another.

The device 1600 can include a groove 1608 formed therein that can be configured to positioned around a pedicle and around a facet joint. The groove 1608 can thus facilitate attachment of the device 1600 to the pedicle without a screw or any other attachment member having to be inserted into the pedicle. Such insertion into the pedicle can exert a force upon the pedicle that can cause damage to the pedicle.

The device 1600 can include a cavity 1638 formed therein that can be configured to couple the device 1600 to second and third connectors (not shown), such as the second connector 1304 and the third connector 1306 of FIG. 35. The device 1600 can include a bore 1640 configured to receive a locking member therein to facilitate secure attachment of the bone connector 1600 and the third connector together in a fixed position relative to one another, similar to the first bore 1316 of FIG. 35.

The device 1600 can include a rod receiving portion configured to movably seat the rod seating member 1602 therein, and hence to movably seat the first rod 1604 therein when the rod seating member 1602 is seating the first rod 1604, as shown for example in FIG. 46. The rod receiving portion can include a bottom surface 1612 configured to movably seat the rod seating member 1602 thereon. The bottom surface 1612 can define a bottom of a cavity 1614 defined by the rod receiving portion. The bottom surface 1612 can, as in this illustrated embodiment, have a concave spherical shape. The spherical shape can allow the rod seating member 1602 seated on the bottom surface 1612 to move polyaxially relative to the device 1600.

The rod seating member 1602 can include a top surface 1618 configured to seat the first rod 1604 thereon. The top surface 1618 can have a concave arcuate shape complementing a typical rod's cylindrical shape. The rod seating member 1602 can include a bottom surface 1616 configured to movably engage the bottom surface 1612 of the device 1600. The rod seating member's bottom surface 1616 can have a shape complementing the shape of the device's bottom surface 1612. Thus, as in this illustrated embodiment, the rod seating member's bottom surface 1616 can have a convex spherical shape. Optionally, the rod seating member's bottom surface 1616 and/or the device's bottom surface 1612 can include a frictional feature configured to improve the engagement between the bottom surfaces 1612, 1616 and/or make the bottom surfaces 1612, 1616 less likely to move relative to one another after locking. Examples of the frictional feature include a roughened surface, a textured surface, and a sticky surface.

The device's cavity 1614 can be configured to receive a locking member 1620 therein to engage the first rod 1604 to facilitate secure attachment of the first rod 1604, the rod seating member 1602, and the device 1600 in a fixed position relative to one another. As in this illustrated embodiment, the locking member 1620 can include a set screw. An interior surface of the device 1600 that defines at least a portion of the cavity 1614, e.g., interior surfaces of opposed arms 1628a, 1628b defining sidewalls of the cavity 1614, can include a corresponding locking element 1622 configured to engage the locking member 1620 and lock the locking member 1620 to the device 1600. As in this illustrated embodiment, the locking element 1622 can include a lip protruding radially inward that can be configured to engage the locking member 1620 in the cavity 1614 so as to prevent upward movement of the locking member 1620, e.g., in a direction away from the bottom surface 1612, when the locking member 1620 and the locking element 1622 are engaged. The opposed arms 1628a, 1628b can be configured to be moved apart from one another in response to a force applied to the device 1600. The movement of the opposed arms 1628a, 1628b apart from one another can facilitate insertion of the locking member 1620 into the cavity 1614 and/or can facilitate locked mating engagement of the locking member 1620 and the locking element 1622. The opposed arms 1628a, 1628b can be pried apart by hand and/or using a surgical tool. The device 1600 can include opposed cut-outs 1636 formed therein, e.g., in the second arm 1628b, which can be configured to engage a surgical tool that facilitates temporary expansion of the cavity 1614 by prying apart the arms 1628a, 1628b, the facilitates seating of the rod 1604 in the cavity 1614, and/or that facilitates installation of the device 1600.

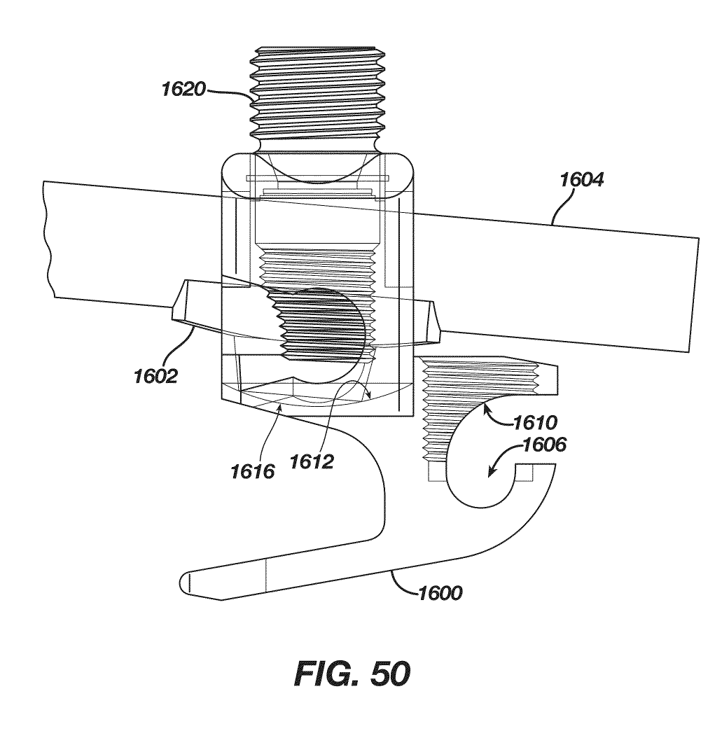

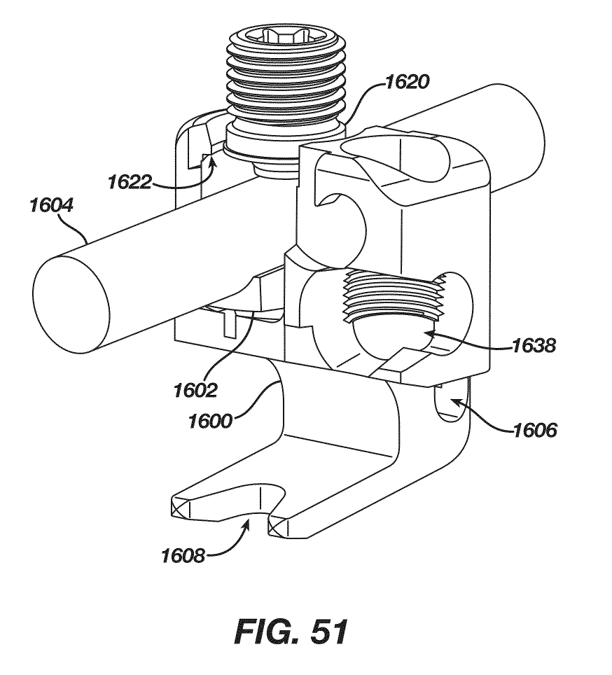

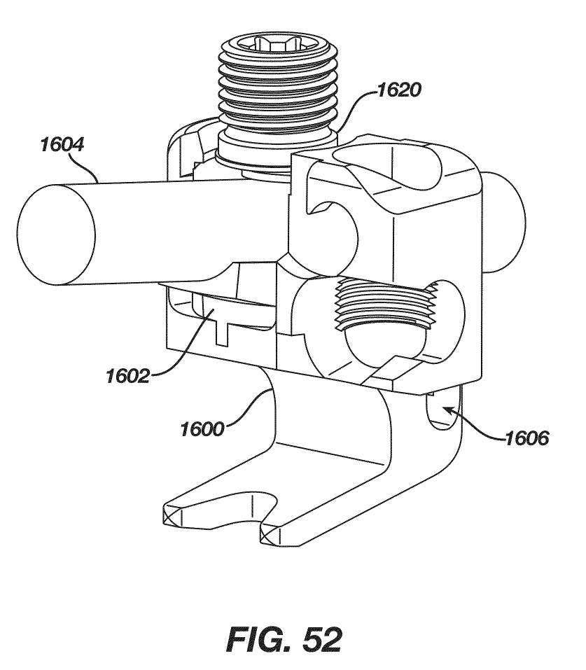

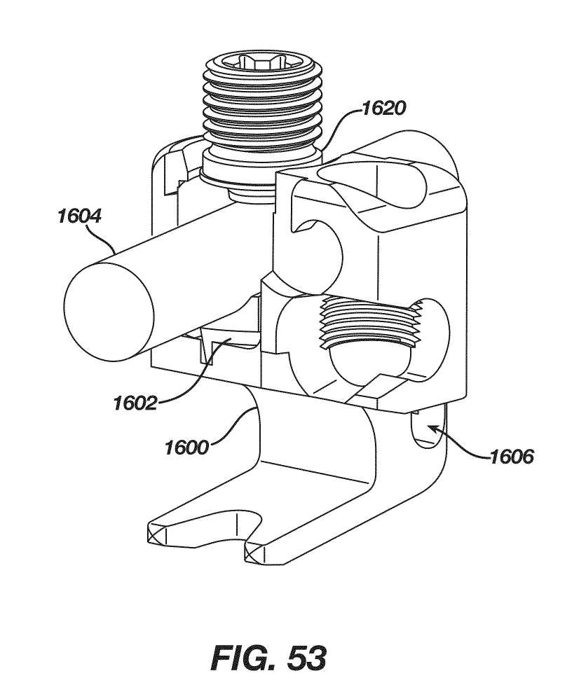

FIG. 46 illustrates the rod seating member 1602 seating the first rod 1604 in the device's rod receiving portion at a zero angle relative to the device 1600. FIG. 49 illustrates the rod seating member 1602 and the first rod 1604 adjusted in position from FIG. 46 relative to the device 1600, with the rod seating member 1602 seating the first rod 1604 at a 10.3° angle relative to the device 1600. FIG. 50 illustrates the rod seating member 1602 and the first rod 1604 adjusted in position from FIG. 46 relative to the device 1600, with the rod seating member 1602 seating the first rod 1604 at a 5° angle relative to the device 1600. FIG. 51 illustrates the rod seating member 1602 and the first rod 1604 adjusted in position from FIG. 46 relative to the device 1600, with the rod seating member 1602 seating the first rod 1604 at a negative angle relative to the device 1600, e.g., with a far end of the first rod 1604 as illustrated in FIG. 51 being higher than a near end of the first rod 1604 as illustrated in FIG. 51. FIG. 52 illustrates the rod seating member 1602 and the first rod 1604 adjusted in position from FIG. 51 relative to the device 1600, with the rod seating member 1602 seating the first rod 1604 at a positive angle relative to the device 1600, e.g., with the near end of the first rod 1604 as illustrated in FIG. 52 being higher than the far end of the first rod 1604 as illustrated in FIG. 52. FIG. 53 illustrates the rod seating member 1602 and the first rod 1604 adjusted in position from FIG. 52 relative to the device 1600, with the rod seating member 1602 seating the first rod 1604 at another negative angle relative to the device 1600, e.g., with the far end of the first rod 1604 as illustrated in FIG. 53 being higher than the near end of the first rod 1604 as illustrated in FIG. 53. FIGS. 49-53 also each illustrate the locking member 1620 locking the rod seating member 1602 seating the first rod 1604 at their respective angular positions.

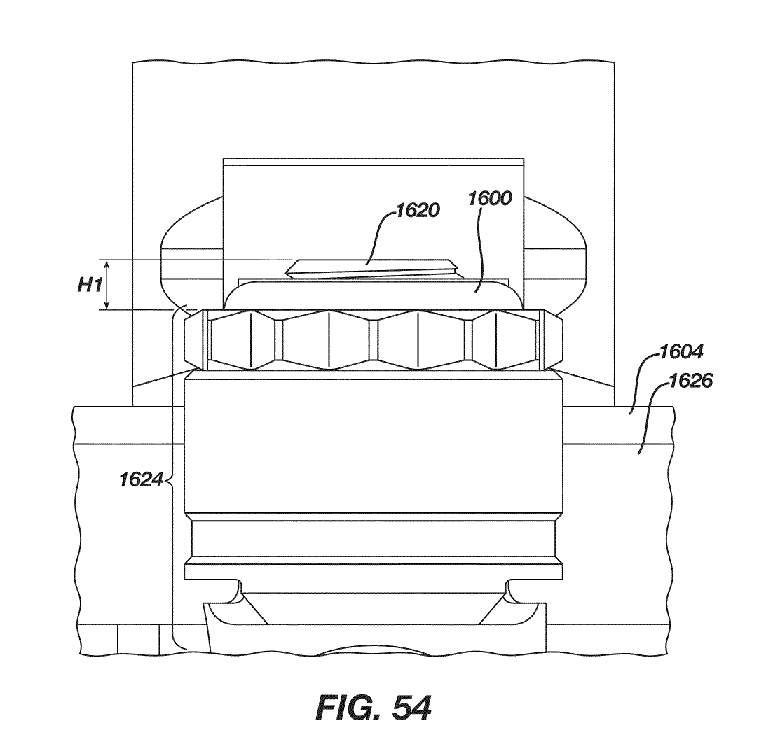

FIG. 54 illustrates the device 1600 coupled to the first rod 1604 behind a traditional device 1624 (in this illustrated embodiment, the Universal Spine System (USS) II Polyaxial available from Synthes, Inc. of West Chester, Pa.) coupled to a spinal rod 1626. The device 1600 can have a higher profile H1 than the traditional device 1624, as shown in FIG. 54.

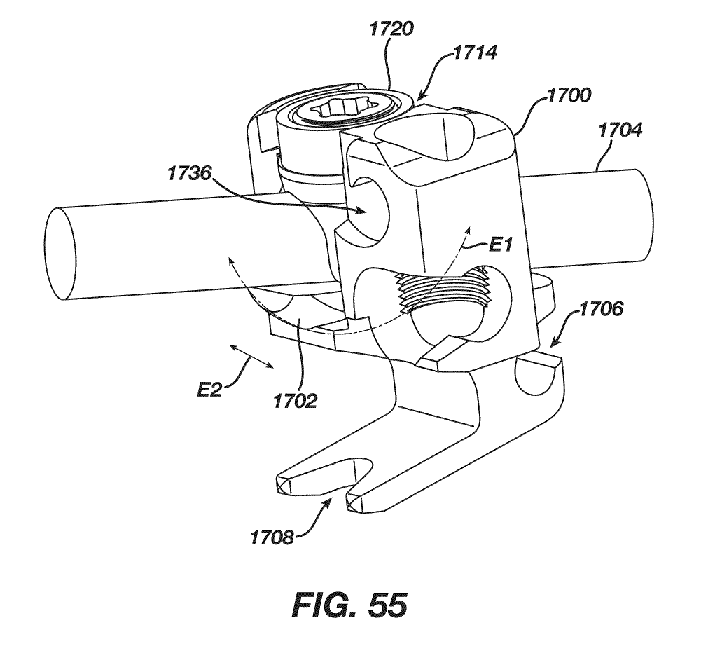

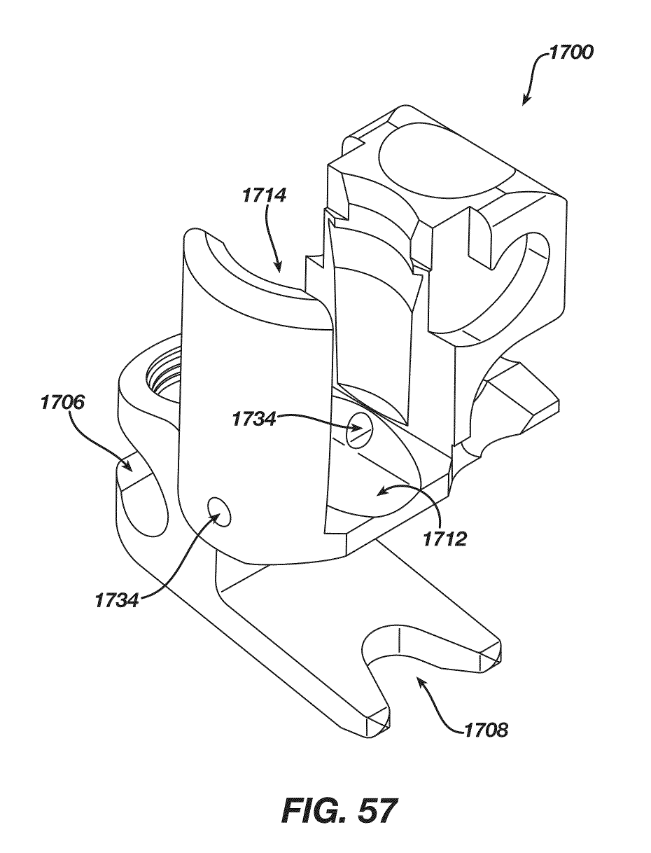

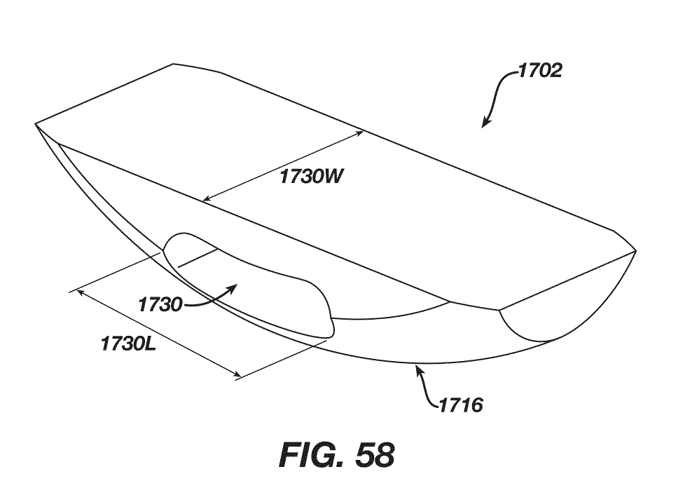

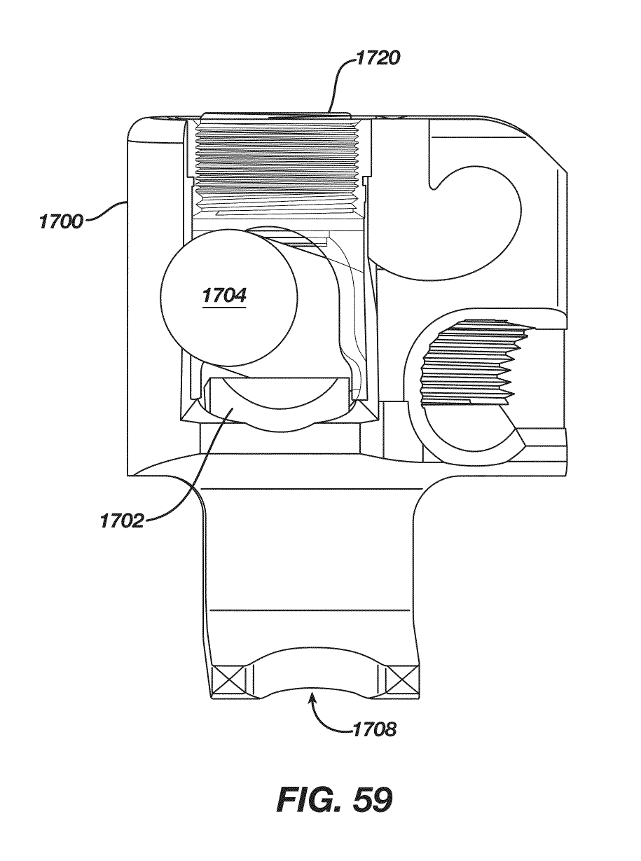

FIGS. 55-57 illustrate another embodiment of a surgical device 1700 including a single bone connector configured to facilitate spinal rod anchoring and to facilitate cross connection. FIGS. 59-63 also illustrate the device 1700. The device 1700 can be configured to allow cross connection to a thoracic vertebra of a patient, going around the patient's transverse process. The device 1700 can generally be configured and used similar to the device 1600 of FIG. 46. The device 1700 can include a cross connector rod seat 1706, can include a bore 1710 configured to receive a locking member therein to engage the second rod to facilitate secure attachment of the second rod and the bone connector 1700 in a fixed position relative to one another, can include a groove 1708 formed therein that can be configured to positioned around a pedicle and around a facet joint, can include a cavity 1738 formed therein that can be configured to couple the device 1700 to second and third connectors 1746, 1748, can include a bore 1740 configured to receive a locking member therein to facilitate secure attachment of the bone connector 1700 and the third connector 1748 together in a fixed position relative to one another, and can be configured to couple to a rod seating member 1702, which is also illustrated in FIGS. 59-63.

The rod seating member 1702 can be configured to seat a first elongate rod 1704 (e.g., a spinal rod) and can be configured to move relative to the bone connector 1700, thereby allowing adjustment of a position of the first rod 1704 relative to the bone connector 1700. In this illustrated embodiment, the rod seating member 1702 is non-removably coupled to the bone connector 1700. The rod seating member 1702 can include an opening 1730 formed therethrough. The opening 1730 can be configured to seat a pin 1732 therein. The pin 1732 and the opening 1730 can cooperate to facilitate movement of the rod seating member 1702 relative to the bone connector 1700, as discussed further below. The device 1700 can have a pin bore 1734 formed therethrough that can be configured to seat the pin 1732 therein. In an exemplary embodiment, the pin 1732 is non-removably fixed in the pin bore 1734, which can help prevent the rod seating member 1702 from being removed from the device 1700.

The device 1700 can include a rod receiving portion configured to movably seat the rod seating member 1702 therein, and hence to movably seat the first rod 1704 therein, as shown for example in FIG. 55. The rod receiving portion can include a bottom surface 1712 configured to movably seat the rod seating member 1702 thereon. The bottom surface 1712 can define a bottom of a cavity 1714 defined by the rod receiving portion. The bottom surface 1712 can, as in this illustrated embodiment, have a concave shape. The concave shape can facilitate movement of the rod seating member 1702 seated on the bottom surface 1712 in two directions E1, E2 relative to the device 1700. The shape of the bottom surface 1712, and the rod receiving member's bottom surface 1716 complementary shape thereto (e.g., a convex shape), can be configured to help limit the rod seating member 1702 to movement in these two directions E1, E2. The first direction E1 can be a back and forth direction, e.g., such that the ends of the first rod 1704 tilt up and down relative to the bone connector 1700. The second direction E2 can be in a side to side direction, e.g., in an X plane as compared to a Y plane of the back and forth movement. The opening 1730 and the pin 1732 can be configured to help limit the rod seating member 1702 to movement in the two directions E1, E2. A longitudinal length 1730L of the opening 1730 can be configured to limit an amount of the rod seating member's and the first rod's movement in the first direction E1. A width 1730w of the opening 1730 can be configured to limit an amount of the rod seating member's and the first rod's movement in the second direction E2.

The device's cavity 1714 can be configured to receive a locking member 1720 therein to engage the first rod 1704 to facilitate secure attachment of the first rod 1704, the rod seating member 1702, and the device 1700 in a fixed position relative to one another. The device 1700 can include a corresponding locking element 1722 configured to engage the locking member 1720 and lock the locking member 1720 to the device 1700. The device 1700 can include opposed cut-outs 1736 formed therein, which can be configured to engage a surgical tool that facilitates temporary expansion of the cavity 1714.

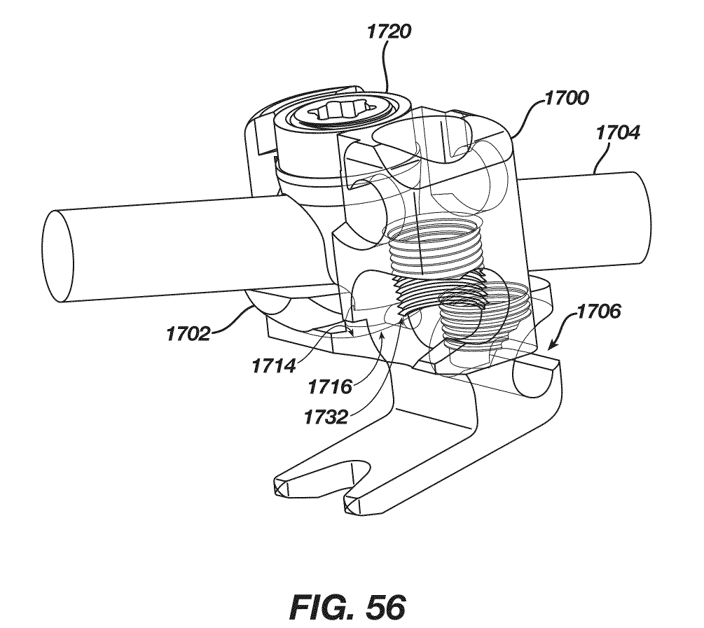

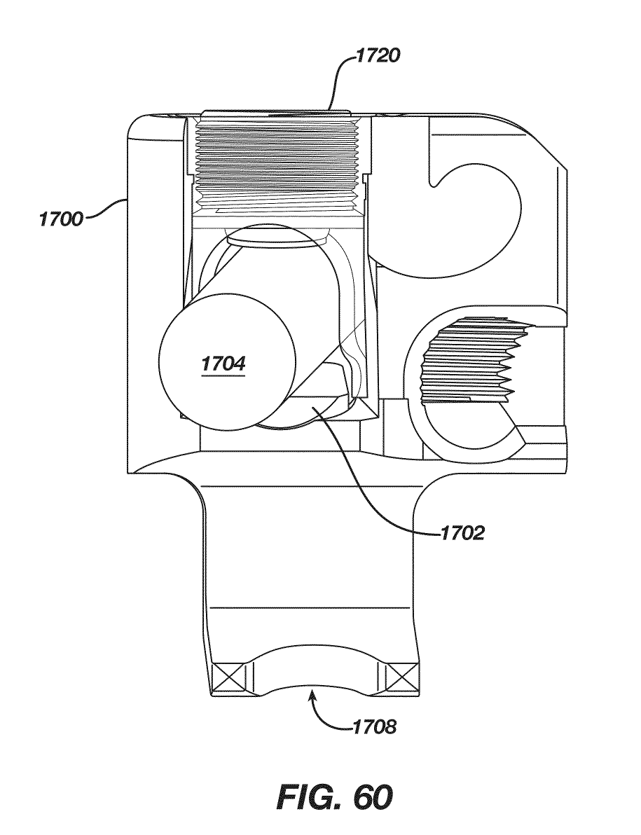

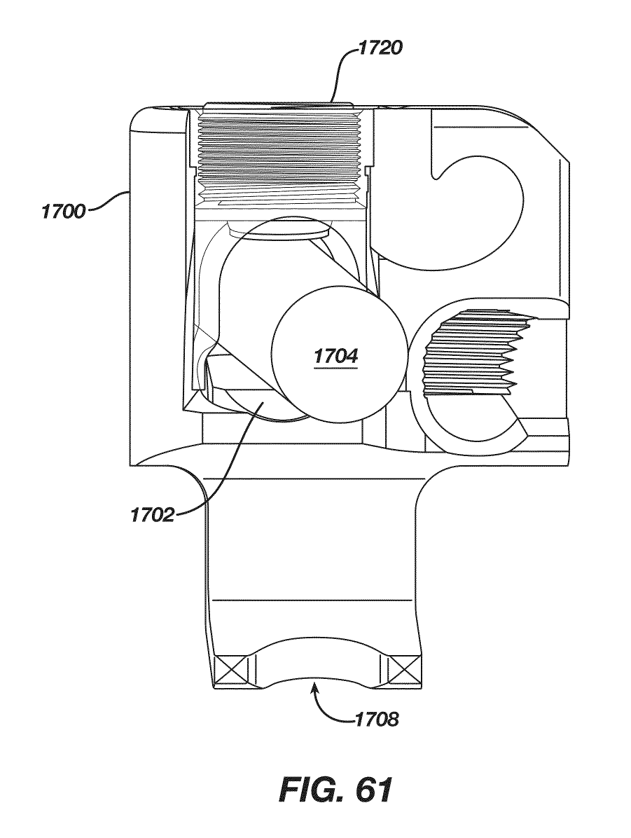

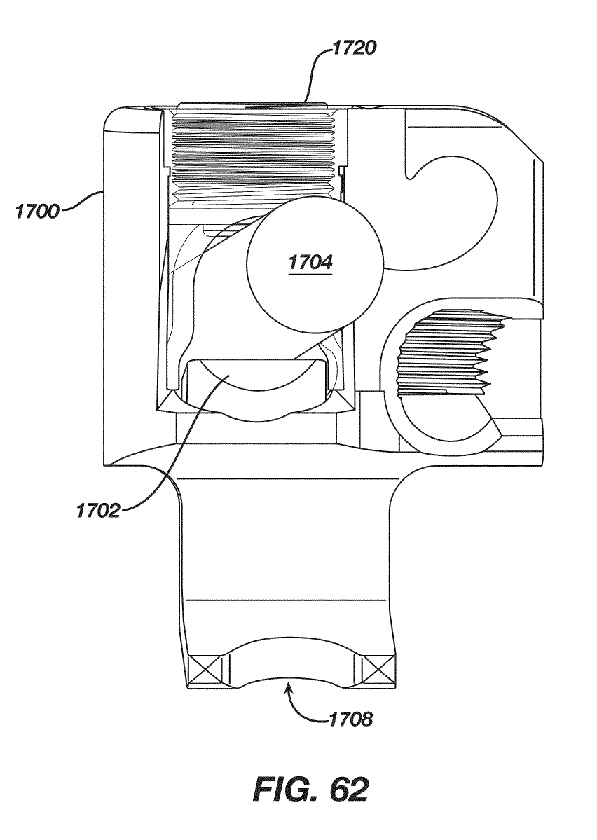

FIGS. 55 and 56 illustrates the rod seating member 1702 seating the first rod 1704 in the device's rod receiving portion at a zero angle relative to the device 1700. FIG. 59 illustrates the rod seating member 1702 and the first rod 1704 adjusted in position from FIGS. 55 and 56 relative to the device 1700, with the rod seating member 1602 seating the first rod 1604 tilted upward in the first direction E1 relative to the device 1700. FIG. 60 illustrates the rod seating member 1702 and the first rod 1704 adjusted in position from FIG. 59 relative to the device 1700, with the rod seating member 1702 seating the first rod 1704 tilted downward in the first direction E1 relative to the device 1700. FIG. 61 illustrates the rod seating member 1702 and the first rod 1704 adjusted in position from FIG. 60 relative to the device 1700, with the rod seating member 1702 seating the first rod 1704 tilted sideways in the second direction E2 relative to the device 1700. FIG. 62 illustrates the rod seating member 1702 and the first rod 1704 adjusted in position from FIG. 61 relative to the device 1700, with the rod seating member 1702 seating the first rod 1704 tilted upward in the first direction E1 relative to the device 1700.

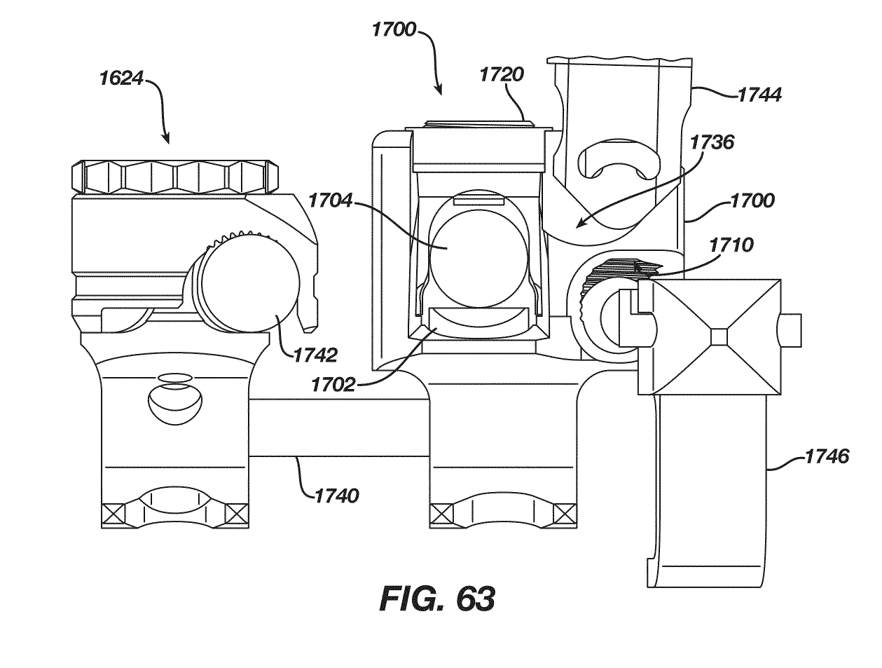

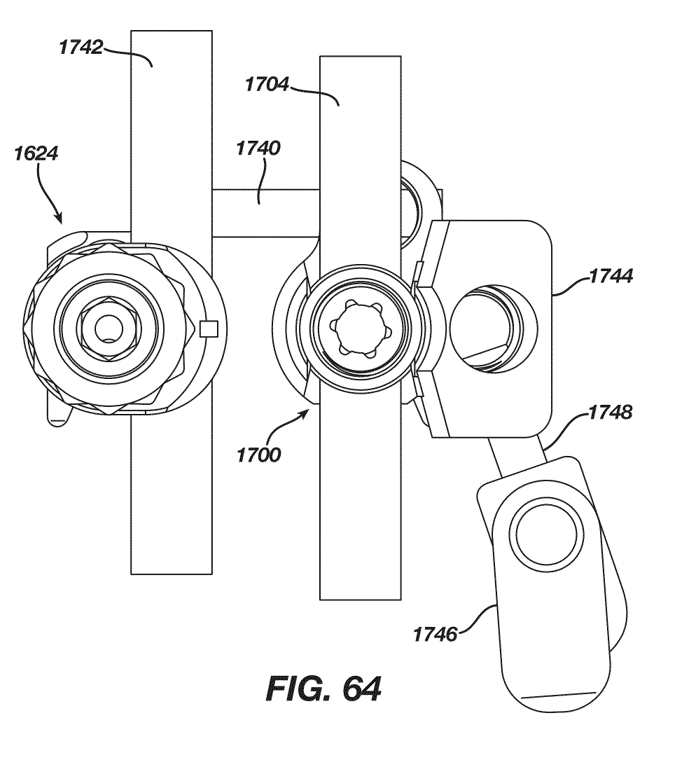

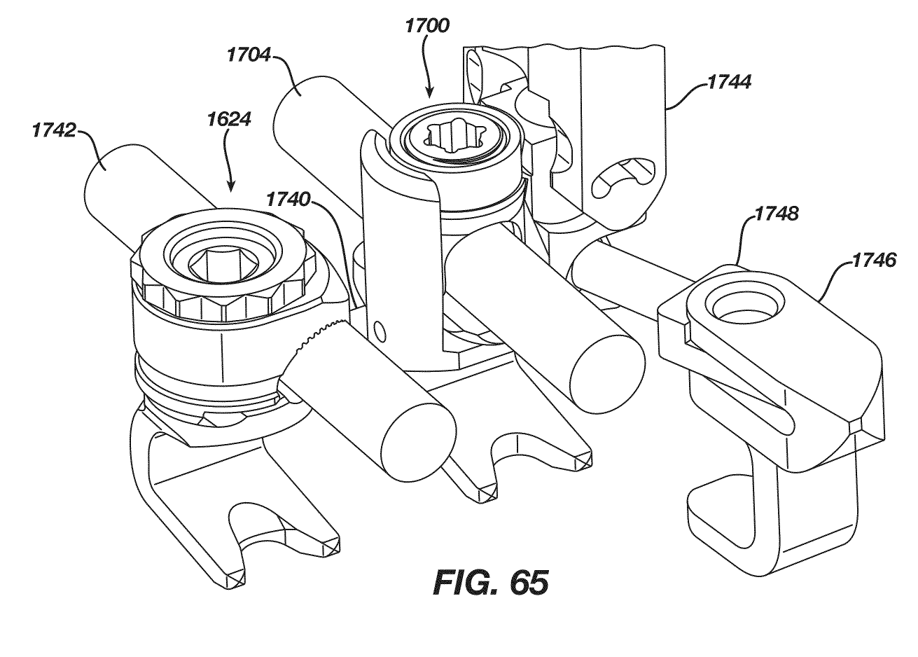

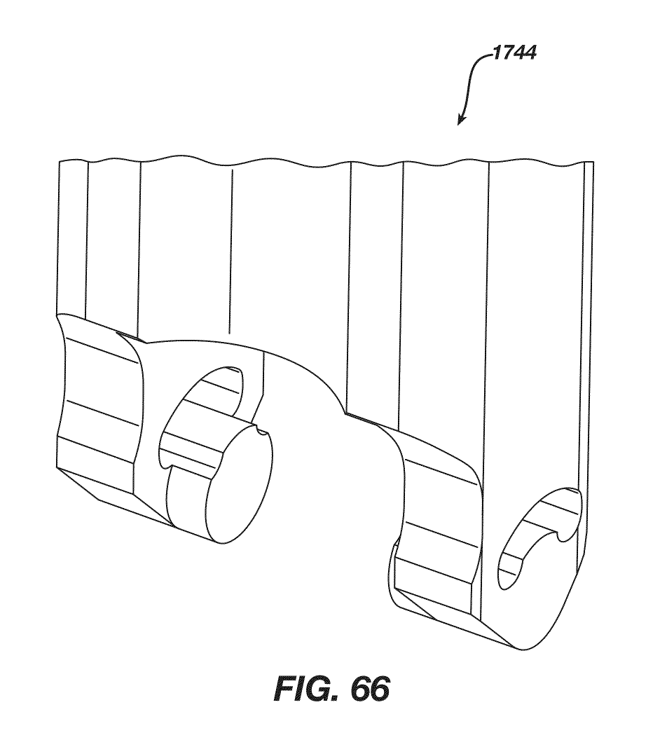

FIGS. 63-65 illustrate the device 1700 coupled to the first rod 1704 and to a second elongate rod 1740 (e.g., a cross connector rod) to the right of the traditional device 1624 of FIG. 54 coupled to the second rod 1740 and coupled to a second spinal rod 1742. The device 1700 can have a higher profile H2 (1.9 mm without the locking member 1720 locked in the device 1700, and 2.5 mm with the locking member 1720 locked in the device 1700) than the traditional device 1624, as shown in FIGS. 63 and 65. FIGS. 63-65 also illustrate an embodiment of an surgical tool 1744 mated to the device's opposed cut-outs 1736 and the second and third connectors 1746, 1748 attached to the bone connector 1700. FIG. 66 also illustrates the surgical tool 1744. The second and third connectors 1746, 1748 in this illustrated embodiment are the same as the second and third connectors 1304, 1306, but other second and third connectors can be used.

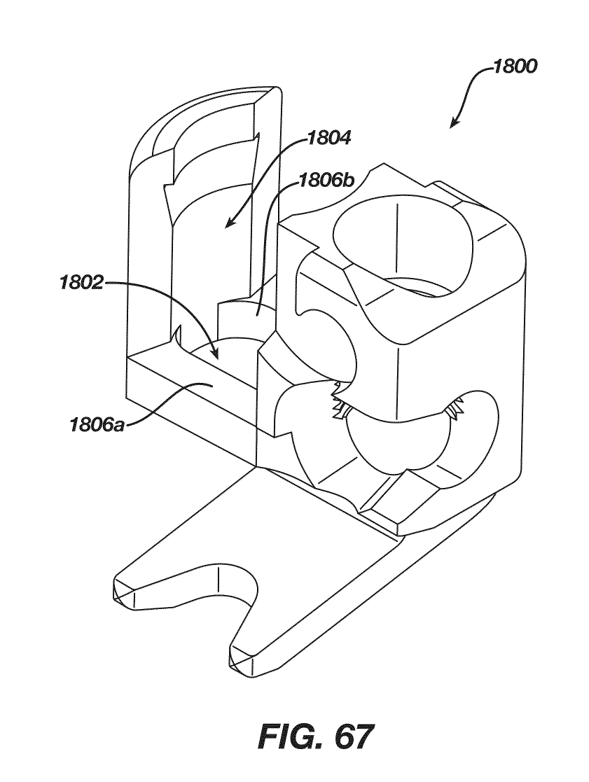

FIG. 67 illustrates another embodiment of a surgical device 1800 including a single bone connector configured to facilitate spinal rod anchoring and to facilitate cross connection. The device 1800 can be configured to allow cross connection to a thoracic vertebra of a patient, going around the patient's transverse process. The device 1800 can generally be configured and used similar to the device 1700 of FIG. 55. However, in this illustrated embodiment, a bottom surface 1802 of a cavity 1804 of the device's rod receiving portion has a different configuration than the bottom surface 1712 of the device 1700, and the device 1800 does not include a pin bore formed therein. The bottom surface 1802 of the cavity 1804 in this illustrated embodiment can extend side to side and be bounded by front and back sidewalls 1806a, 1806b so as to be configured to retain a rod seating member therein and allow movement thereof relative to the device 1800.

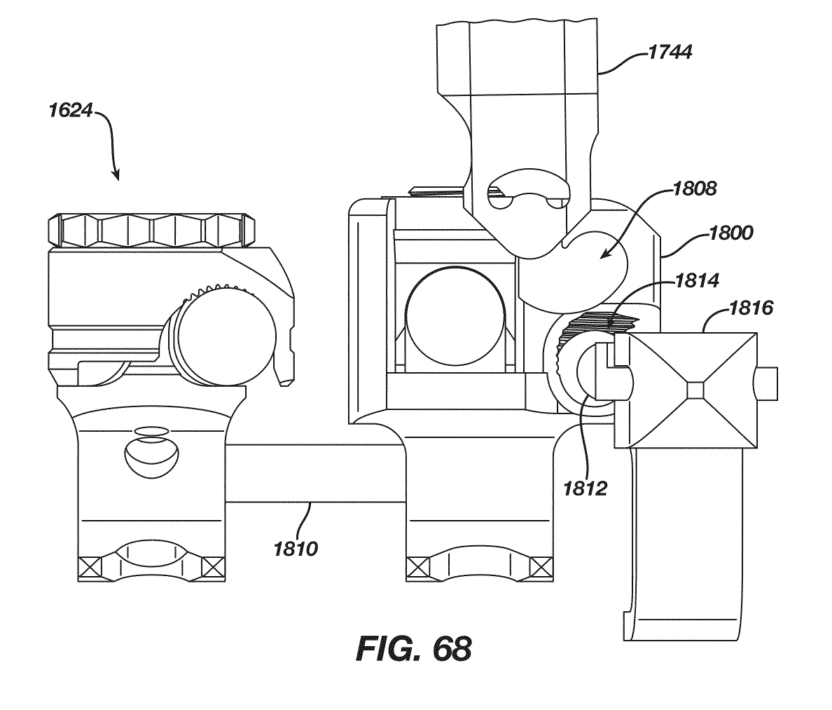

FIGS. 68-78 illustrate an embodiment of a range of movement of the surgical tool 1744 of FIG. 66 relative to the device 1800 of FIG. 67 with the tool 1744 mated to opposed cut-outs 1808 formed in the device 1800 (one of the opposed cut-outs is obscured in FIGS. 68-78). The tool 1744 is only an illustrative example; another surgical tool can be used similar to the surgical tool 1744. FIGS. 68-78 illustrate the device 1800 with a second elongate rod 1810 (e.g., a cross connector rod) coupled thereto by being seated in a cross connector seat (obscured in FIGS. 68-78), with a third connector 1812 coupled to the device 1800 with one end of the third connector 1812 being polyaxially seated in a cavity 1814 of the device 1800, and the other end of the third connector 1812 coupled to a second connector 1816. FIGS. 68-78 also illustrate the second rod 1810 coupled to the traditional device 1624 of FIG. 54.

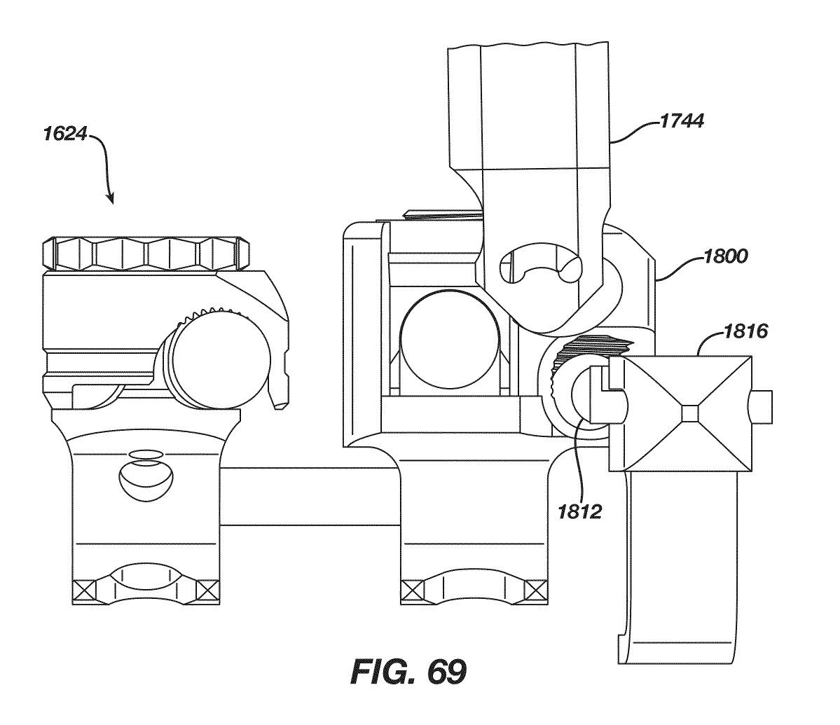

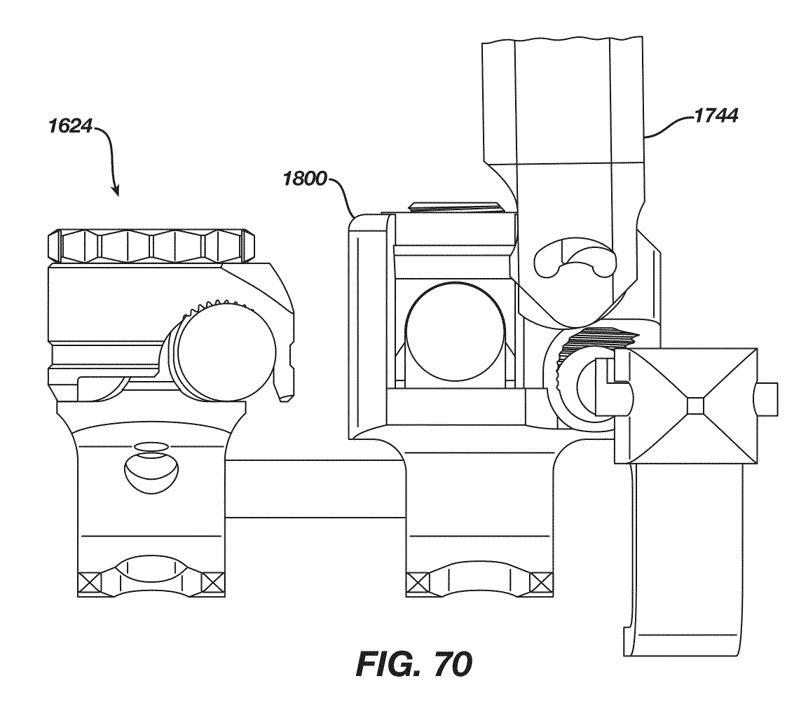

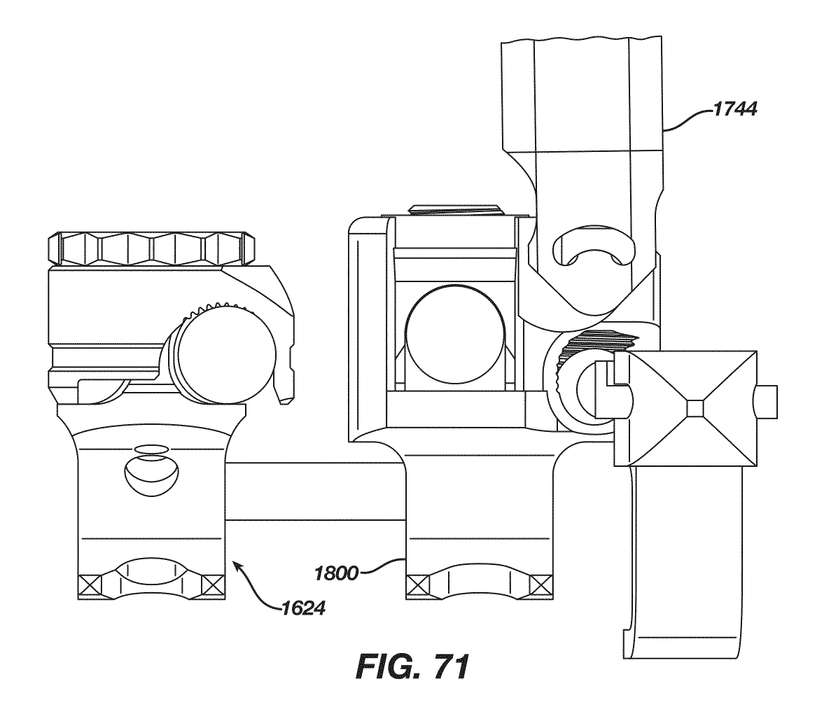

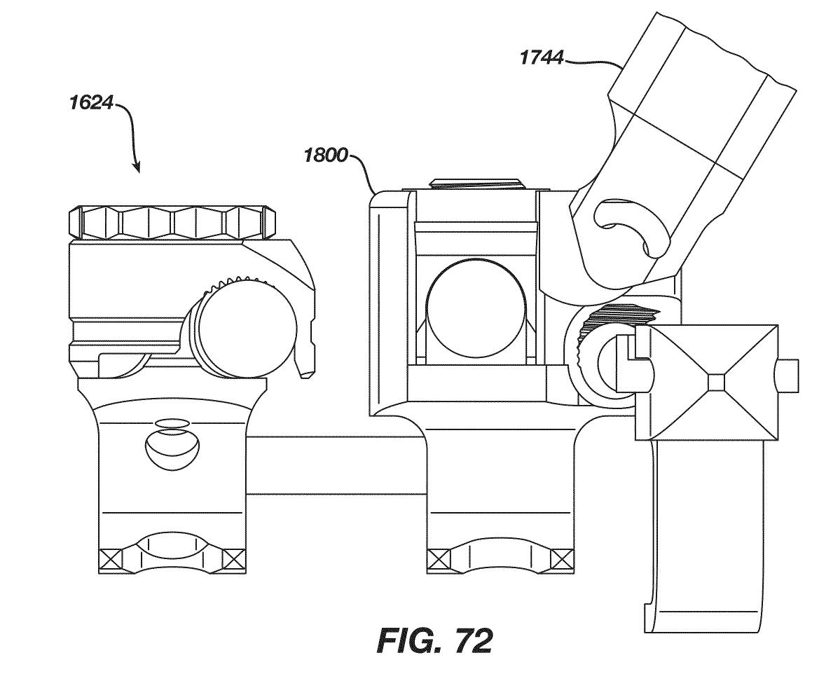

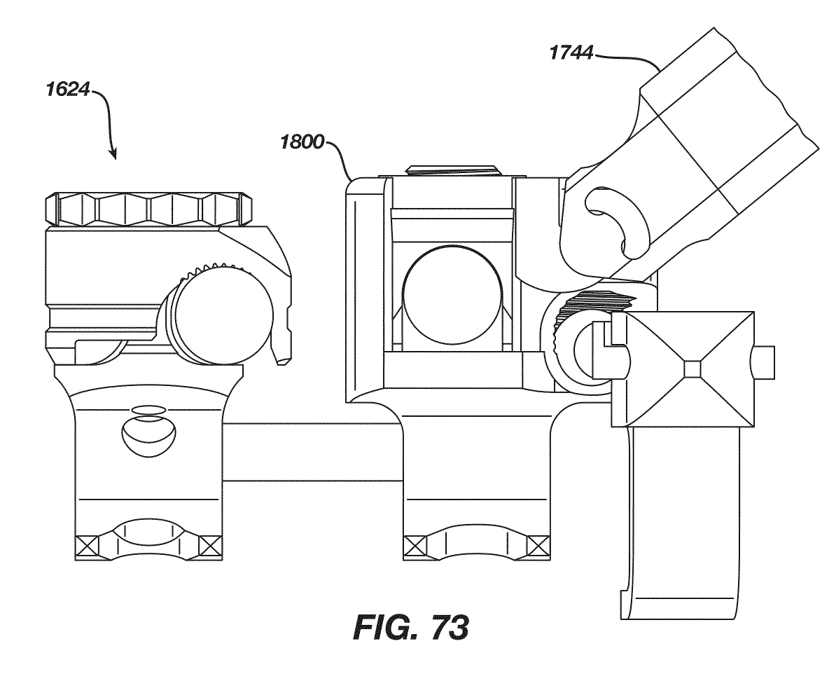

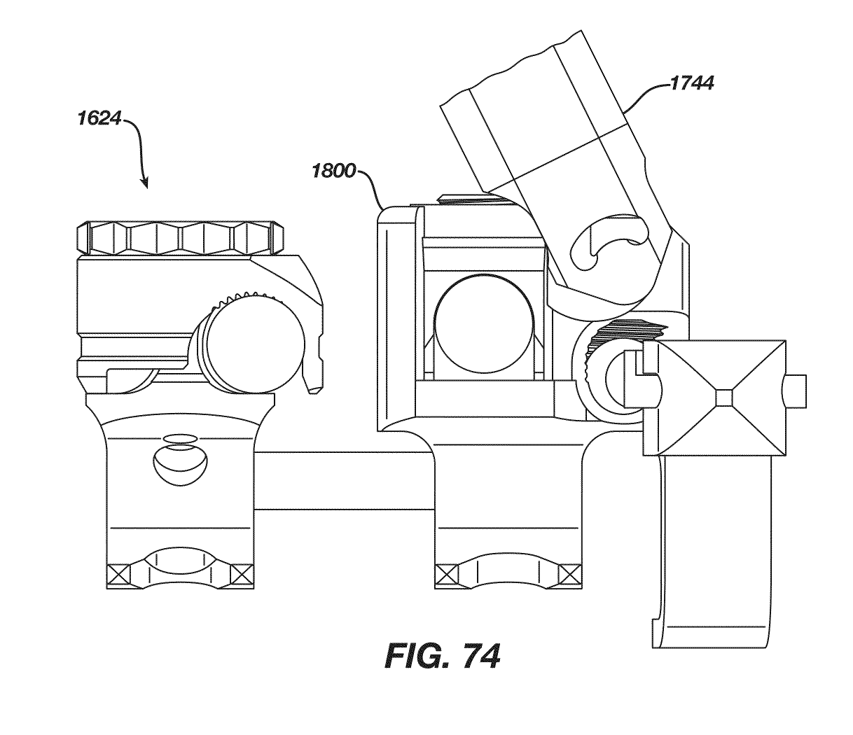

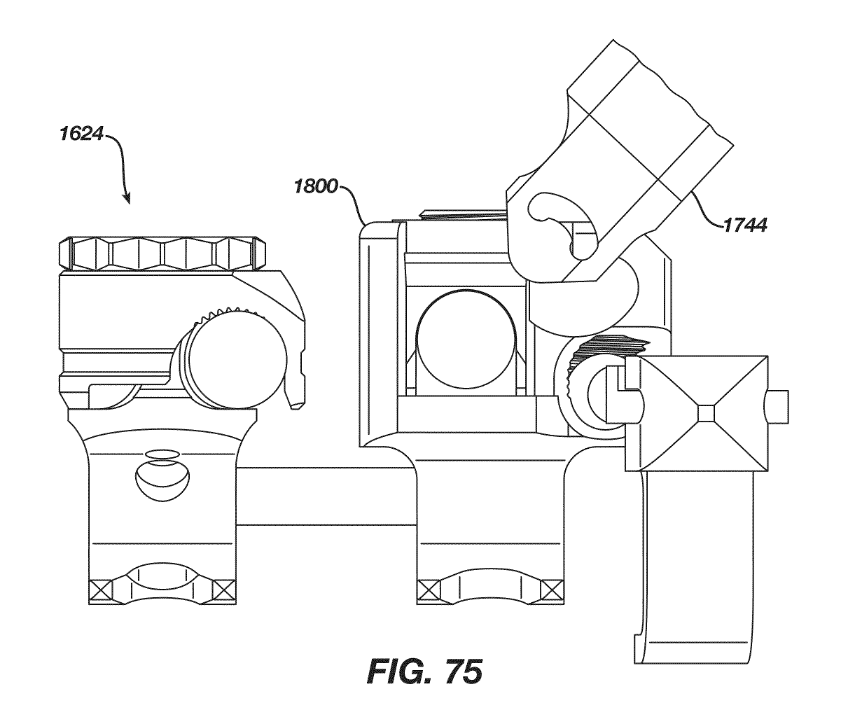

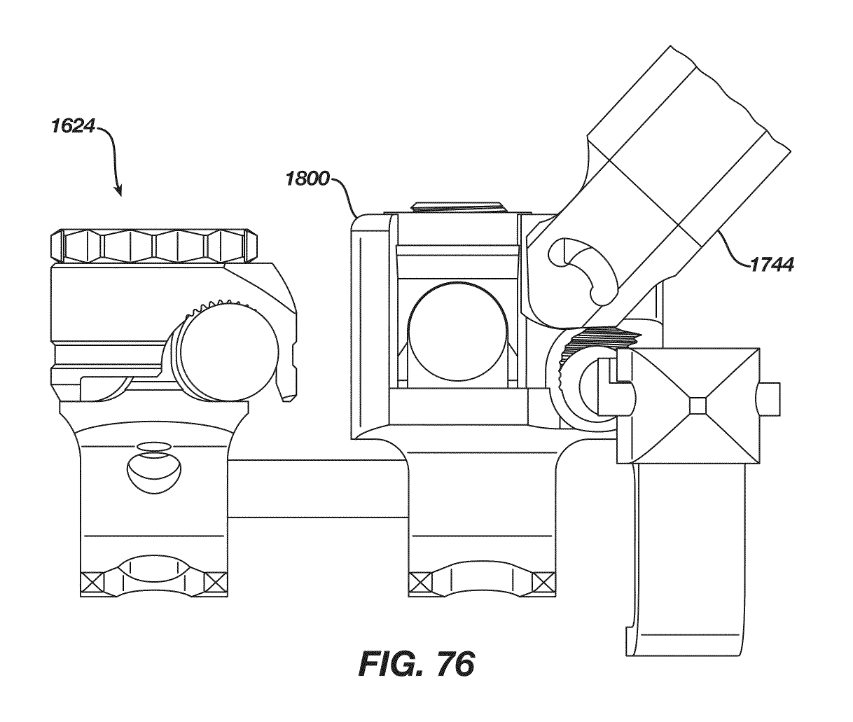

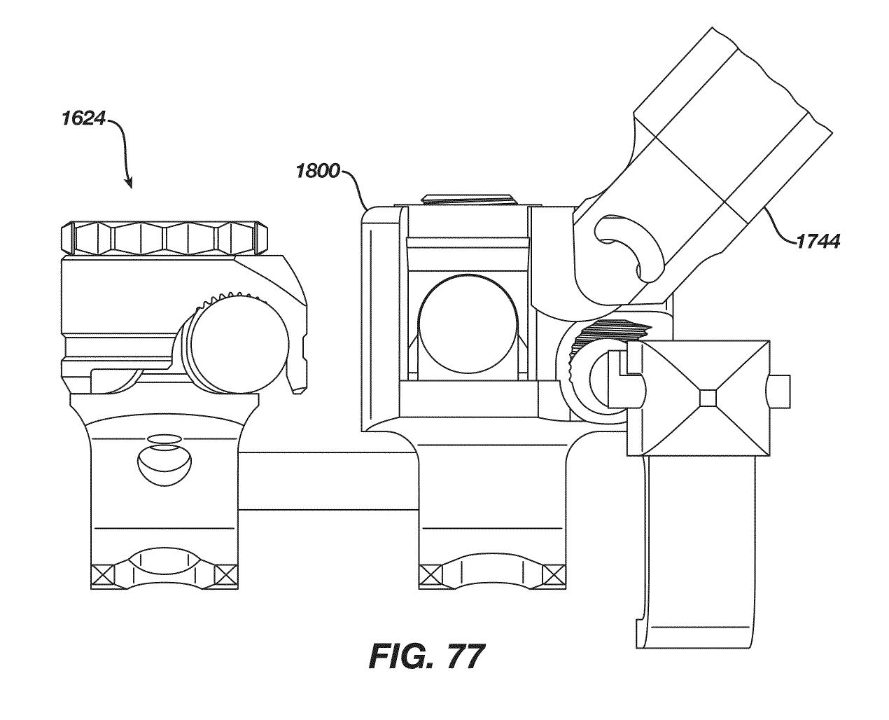

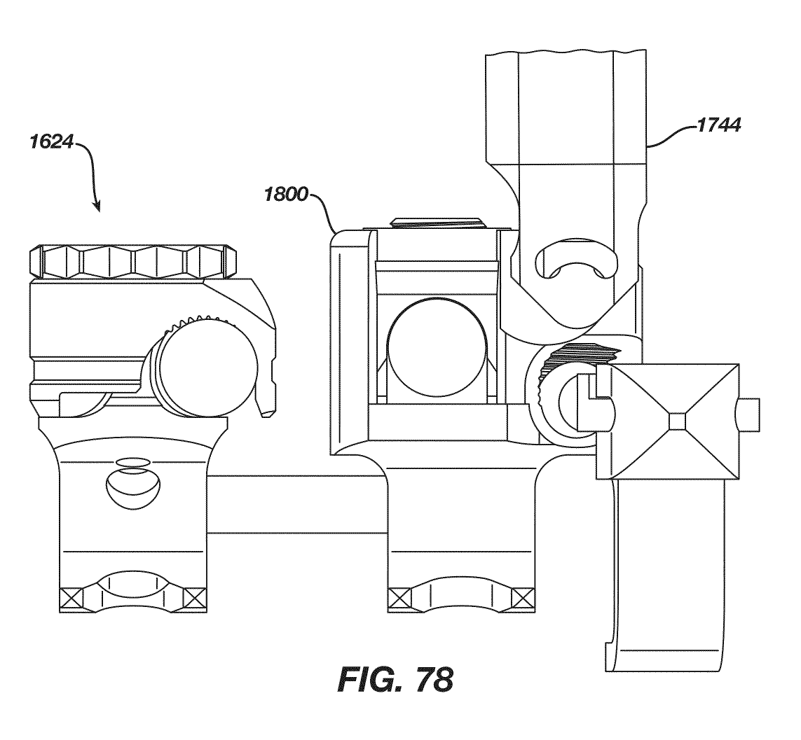

FIG. 68 illustrates the tool 1744 engaged with an upper region of an elongate portion of the cut-outs 1808. FIG. 69 illustrates the tool 1744 adjusted in position from FIG. 68 relative to the device 1800, with the tool 1744 engaging a bottom region of the elongate portion of the cut-outs 1808. FIG. 70 illustrates the tool 1744 adjusted in position from FIG. 69 relative to the device 1800, with the tool 1744 engaging a first side region of a curved, bulbous portion of the cut-outs 1808. FIG. 71 illustrates the tool 1744 adjusted in position from FIG. 70 relative to the device 1800, with the tool 1744 engaging a second, opposite side region of the curved, bulbous portion of the cut-outs 1808. FIG. 72 illustrates the tool 1744 adjusted in position from FIG. 71 relative to the device 1800, with the tool 1744 still engaging the second, opposite side region of the curved, bulbous portion of the cut-outs 1808 but being rotated, e.g., longitudinally angled, 8n a first direction relative to its position in FIG. 71. FIG. 73 illustrates the tool 1744 adjusted in position from FIG. 72 relative to the device 1800, with the tool 1744 still engaging the second, opposite side region of the curved, bulbous portion of the cut-outs 1808 but being rotated in the first direction relative to its position in FIG. 72. FIG. 74 illustrates the tool 1744 adjusted in position from FIG. 73 relative to the device 1800, with the tool 1744 still engaging the second, opposite side region of the curved, bulbous portion of the cut-outs 1808 but being rotated in a second direction, which is opposite to the first direction, relative to its position in FIG. 73. FIG. 75 illustrates the tool 1744 adjusted in position from FIG. 74 relative to the device 1800, with the tool 1744 engaging the upper region of the elongate portion of the cut-outs 1808 and being rotated in the first direction relative to its position in FIG. 74. FIG. 76 illustrates the tool 1744 adjusted in position from FIG. 75 relative to the device 1800, with the tool 1744 engaging the lower region of the elongate portion of the cut-outs 1808 relative to its position in FIG. 75. FIG. 77 illustrates the tool 1744 adjusted in position from FIG. 76 relative to the device 1800, with the tool 1744 engaging the second side region of the curved, bulbous portion of the cut-outs 1808 relative to its position in FIG. 76. FIG. 78 illustrates the tool 1744 adjusted in position from FIG. 77 relative to the device 1800, with the tool 1744 still engaging the second side region of the curved, bulbous portion of the cut-outs 1808 but being rotated in the second direction relative to its position in FIG. 77.

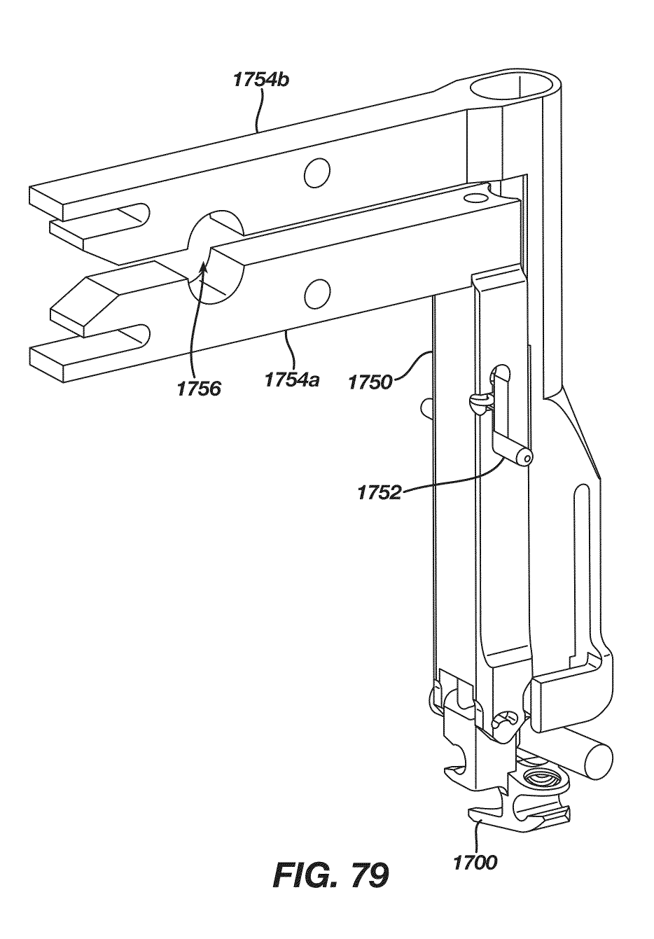

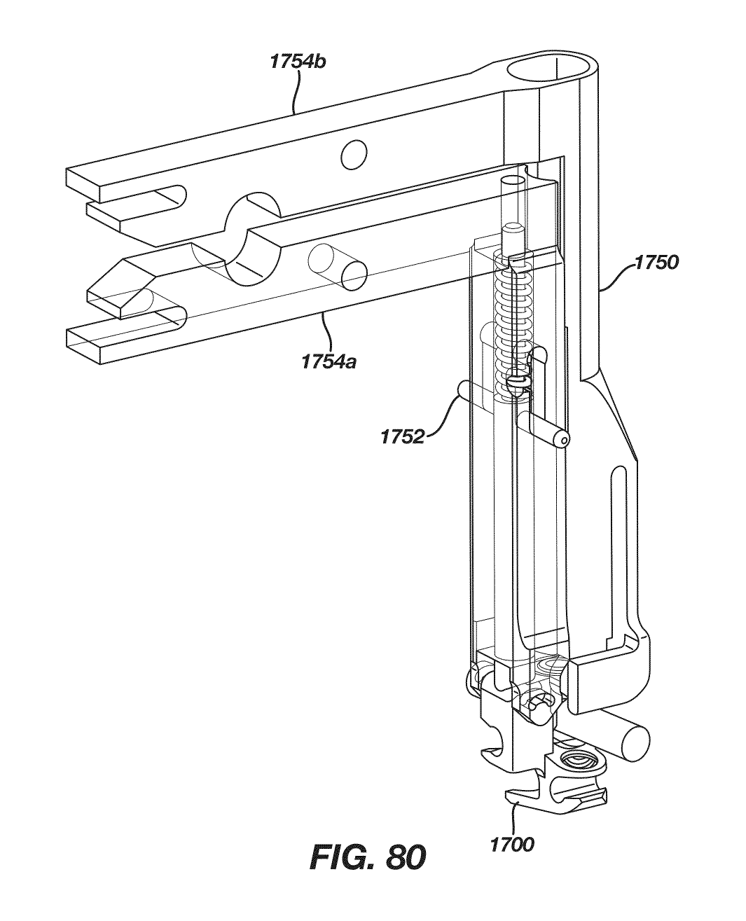

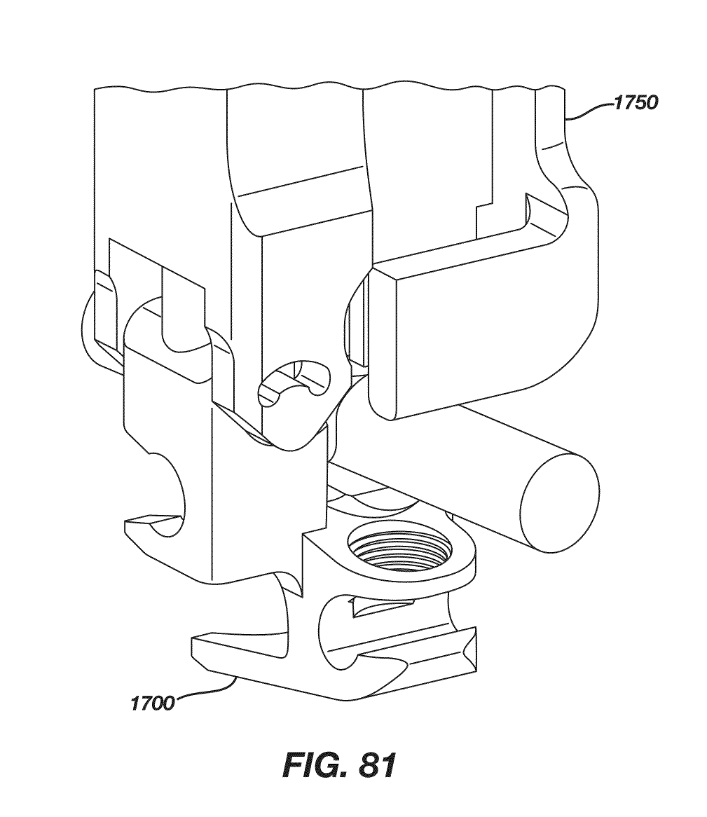

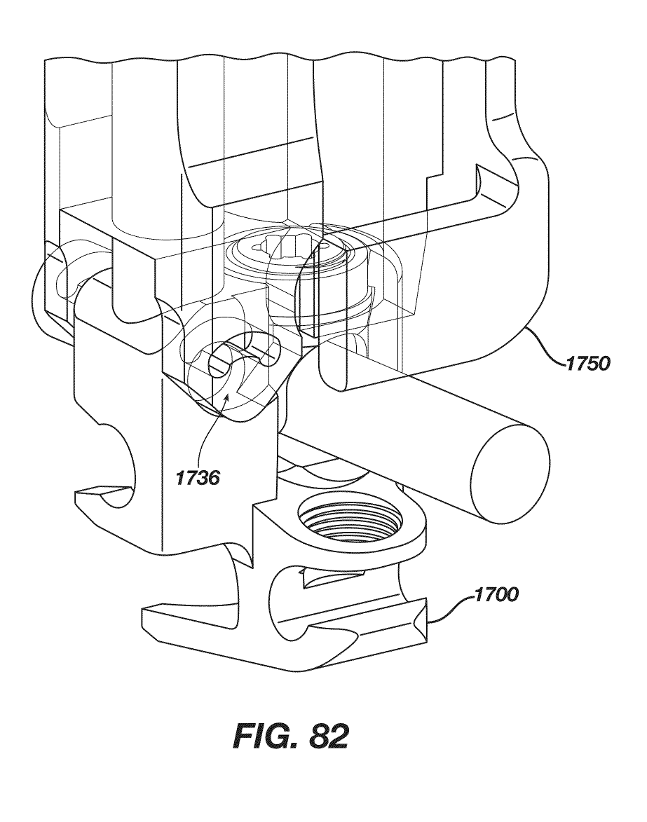

FIGS. 79-82 illustrate an embodiment of a range of movement of another embodiment of a surgical tool 1750, which can be generally configured and used similar to the surgical tool 1744 of FIG. 66, relative to the device 1700 of FIG. 55 with the tool 1750 mated to the opposed cut-outs 1736 formed in the device 1700. The tool 1750 can be configured to install an elongate rod 1752 relative to the device 1700. The tool 1750 can include a pair of levers 1754a, 1754b configured to move in parallel lever action by pivoting at a hinge joint (not shown) connecting the two levers 1754a, 1754b together at a region 1756. A handle (not shown) can be attached to the levers 1754a, 1754b, which can facilitate manual pivoting of the levers 1754a, 1754b. FIGS. 81 and 82 show a close-up view of a connection area between the tool 1750 and the device 1700.

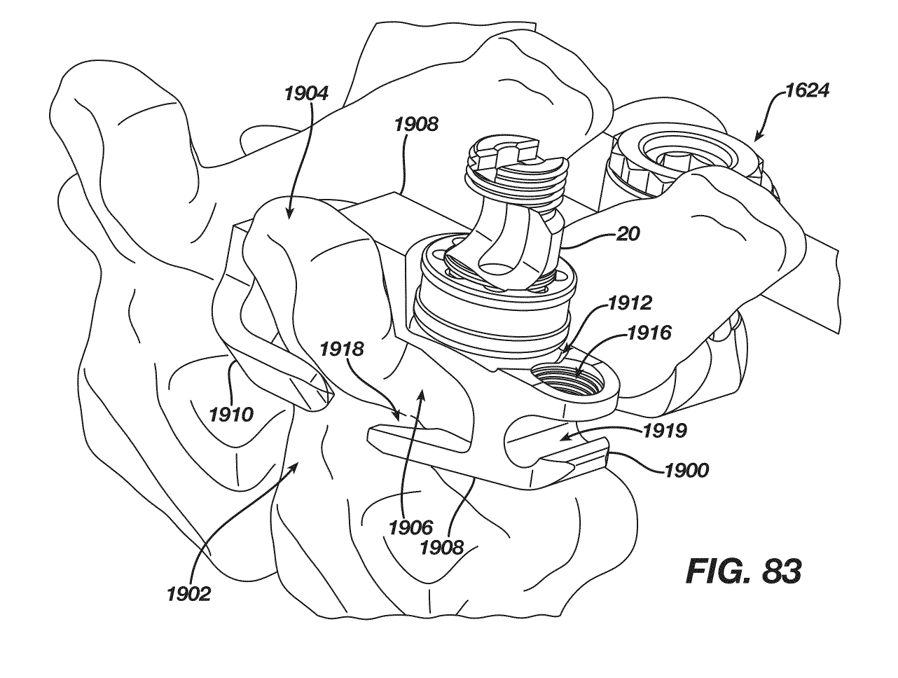

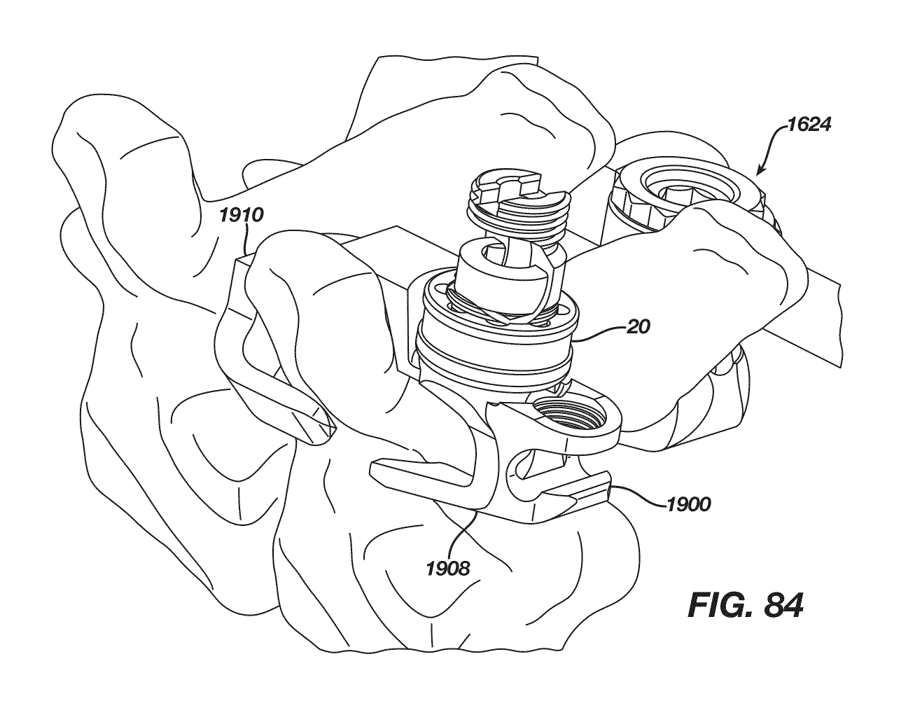

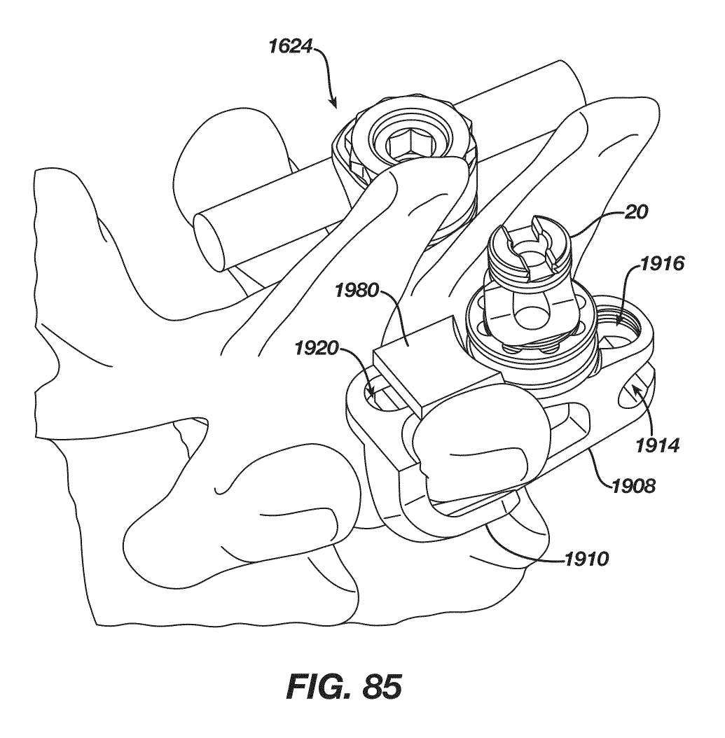

FIGS. 83-85 illustrate another embodiment of a surgical device 1900 including a bone connector configured to facilitate spinal rod anchoring, configured to facilitate cross connection, and configured to couple to the side-loading polyaxial receiver member 20 of FIG. 26. The device 1900 can be configured to allow cross connection to a thoracic vertebra 1902 of a patient, going around the patient's transverse process 1904. FIGS. 83-85 illustrate the device attached to a pedicle 1906 of the patient. For comparison purposes, FIGS. 83-85 also illustrate the traditional device 1624 of FIG. 54 attached to the patient. The device 1900 in this illustrated embodiment can have a profile about 4 mm higher than the traditional device 1624, e.g., because of the polyaxial first rod capability.

As in this illustrated embodiment, the device 1900 can include a first connector 1908 and a second connector 1910 configured to couple to the first connector 1908. The first connector 1908 can be configured to couple to a first elongate rod (not shown), e.g., a spinal rod, via the receiver member 20, and can be configured to couple to a second elongate rod (not shown), e.g., a cross connector rod, via seating in a cross connector seat 1914. The first connector 1908 can include a first bore 1912 configured to receive the receiver member 20 therein, a second bore 1916 configured to receive a locking member therein to engage the second rod to facilitate secure attachment of the second rod and the first connector 1908 in a fixed position relative to one another, a third bore (obscured in FIGS. 83-85) configured to facilitate secure attachment of the first and second connectors 1908, 1910 together in a fixed position relative to one another, and a groove 1918 formed therein that can be configured to positioned around the pedicle 1906. The second connector 1910 can include a bore 1920 formed therethrough and configured to align with the third bore to facilitate secure attachment of the first and second connectors 1908, 1910 together in a fixed position relative to one another. The second connector 1910 can be configured to help hold the first connector 1908 in position relative to the vertebra 1902 by extending around the transverse process 1904. Alternatively, the second connector 1910 can extend around the patient's lamina.

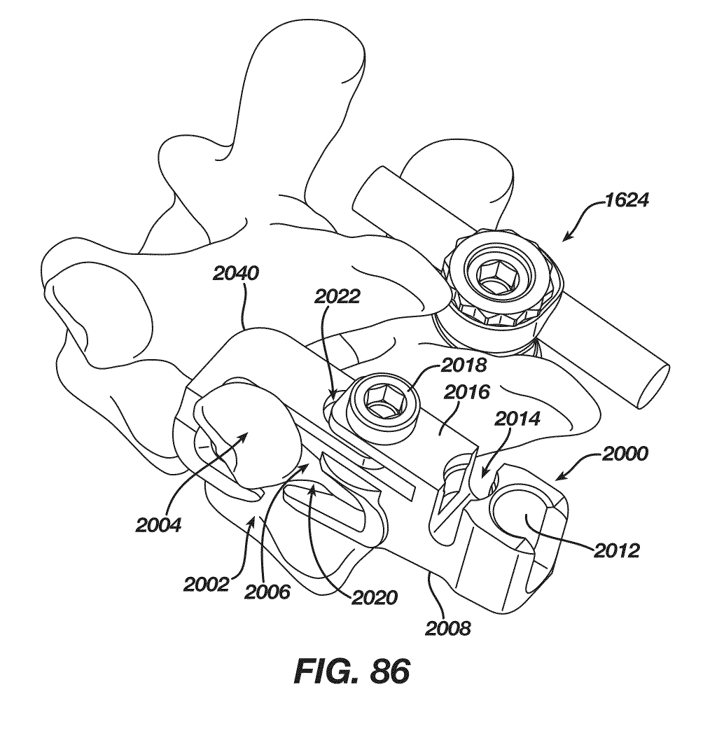

FIG. 86 illustrates another embodiment of a surgical device 2000 including a bone connector configured to facilitate spinal rod anchoring and configured to facilitate cross connection. The device 2000 can be configured to allow cross connection to a thoracic vertebra 2002 of a patient, going around the patient's transverse process 2004. The device 2000 can generally be configured and used similar to the device 1900 of FIG. 83. FIG. 86 illustrates the device attached to a pedicle 2006 of the patient. For comparison purposes, FIG. 86 also illustrates the traditional device 1624 of FIG. 54 attached to the patient. The device 1900 in this illustrated embodiment can have a profile less than the traditional device 1624.

As in this illustrated embodiment, the device 2000 can include a first connector 2008 and a second connector 2010 configured to couple to the first connector 2008. The first connector 2008 can include a rod seat 2012 configured to couple to a first elongate rod (not shown), e.g., a spinal rod, which can be polyaxially seated therein similar to the third connector 1812 being polyaxially seated in a cavity 1814 of the device 1800 of FIG. 68. The first connector 2008 can be configured to receive a first locking member (not shown) in the rod seat 2012 to facilitate secure attachment of the first rod and the first connector 2008 in a fixed position relative to one another. The first connector can include a cross connector seat 2014 that can be configured to seat a second elongate rod (not shown), e.g., a cross connector rod, therein. The first connector 2008 can be configured to receive a second locking member (not shown) in the cross connector seat 2014 to facilitate secure attachment of the second rod and the first connector 2008 in a fixed position relative to one another. The first cross connector can include a bore 2016 configured to receive a third locking member 2018 therein to facilitate secure attachment of the first and second connectors 2008, 2010 together in a fixed position relative to one another, and a groove 2020 formed therein that can be configured to positioned around the pedicle 2006. The second connector 1910 can include an opening 2022 formed therethrough and configured to align with the bore 2016 to facilitate secure attachment of the first and second connectors 2008, 2010 together in a fixed position relative to one another. The second connector 2010 can be configured to help hold the first connector 2008 in position relative to the vertebra 2002 by extending around the transverse process 2004. Alternatively, the second connector 2010 can extend around the patient's lamina.

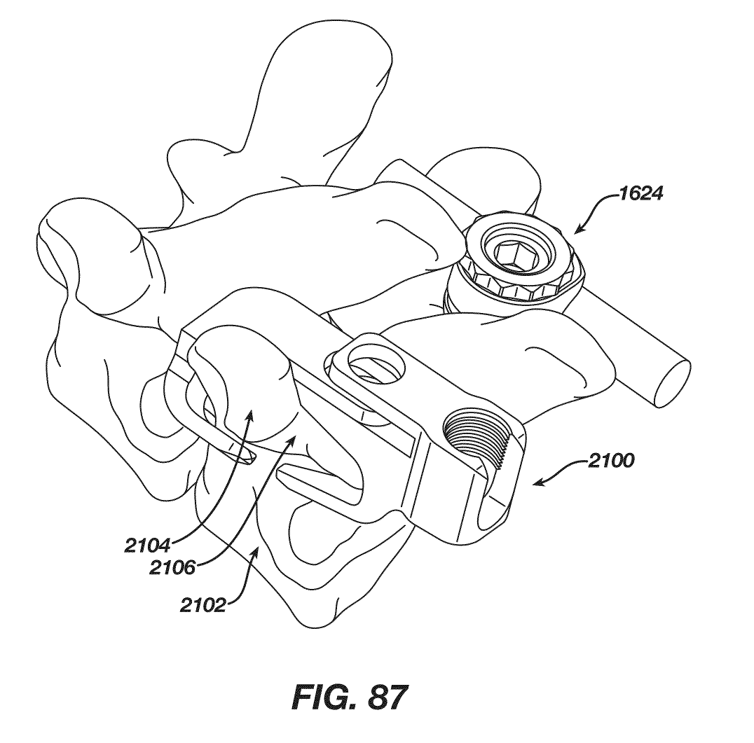

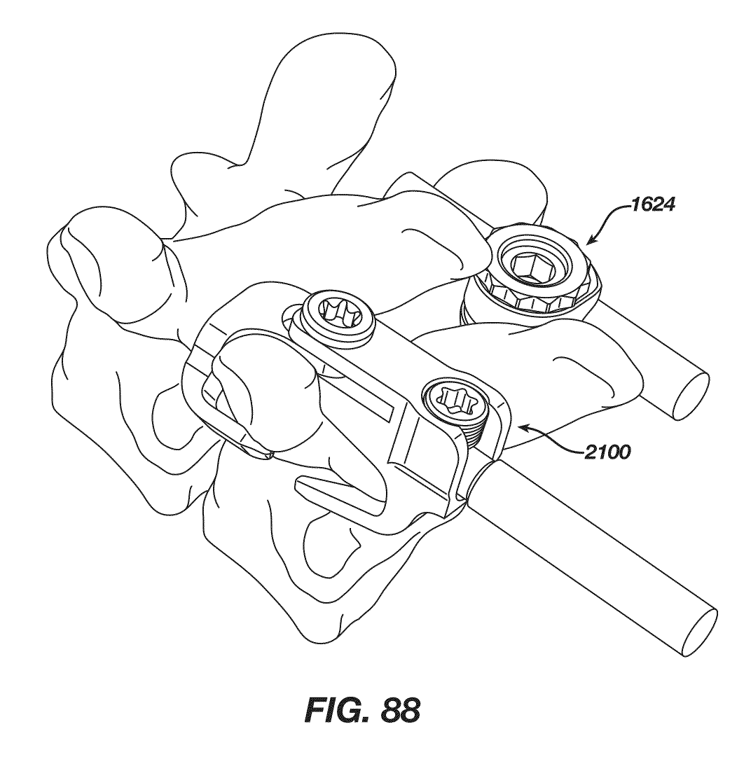

FIGS. 87 and 88 illustrate another embodiment of a surgical device 2100 including a bone connector configured to facilitate spinal rod anchoring. The device 2100 can be configured to go around a transverse process 2104 of the patient's vertebra 2102. The device 2100 can generally be configured and used similar to the device 2000 of FIG. 86. However, unlike the device 2000, in this illustrated embodiment, the device 2100 does not include a cross connector seat.

FIGS. 87 and 88 illustrate the device attached to a pedicle 2106 of the patient. For comparison purposes, FIGS. 87 and 88 also illustrate the traditional device 1624 of FIG. 54 attached to the patient. The device 2100 in this illustrated embodiment can have a profile less than the traditional device 1624.

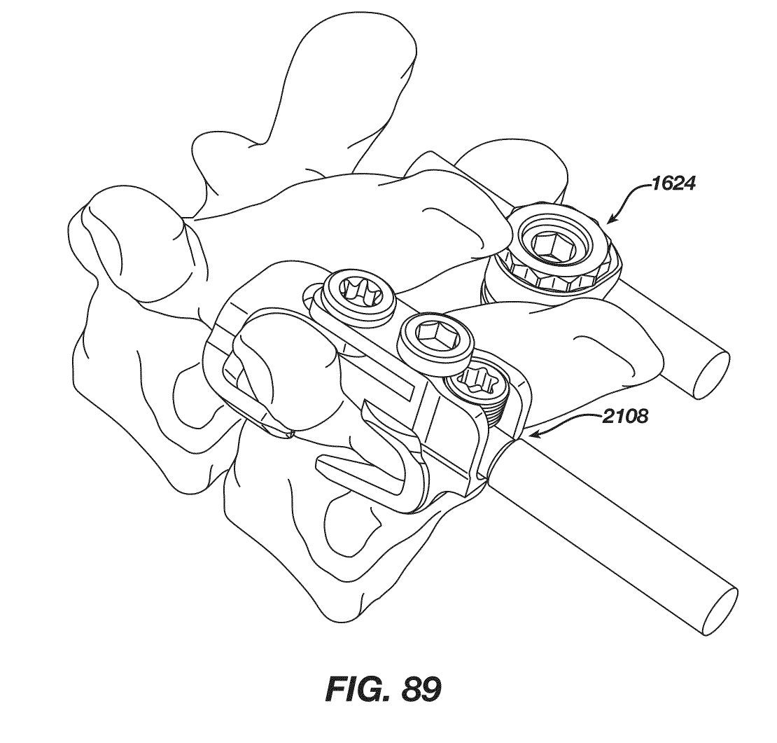

For comparison purposes with the embodiments of the devices 2000, 2100 of FIGS. 86 and 87, respectively, FIG. 89 illustrates another traditional device 2108 attached to a pedicle 2110. The devices 2000, 2100 of FIGS. 86 and 87 can each have a lower profile than this traditional device 2108. For further comparison purposes, FIG. 89 also illustrates the traditional device 1624 of FIG. 54.

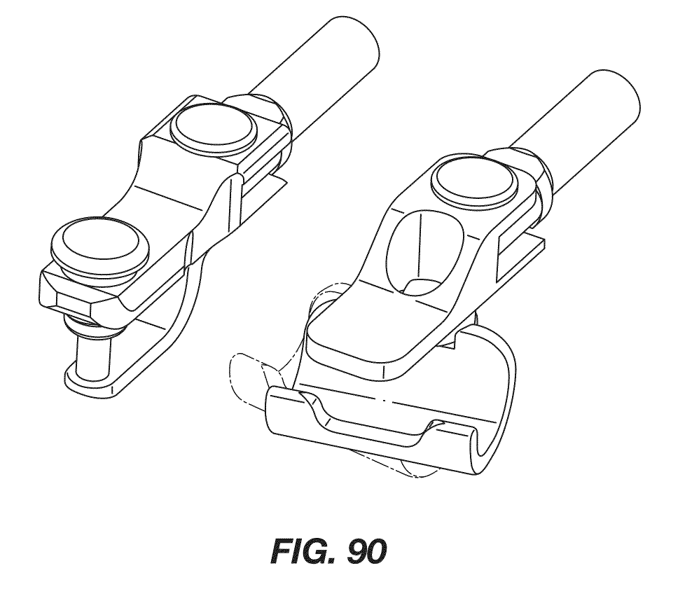

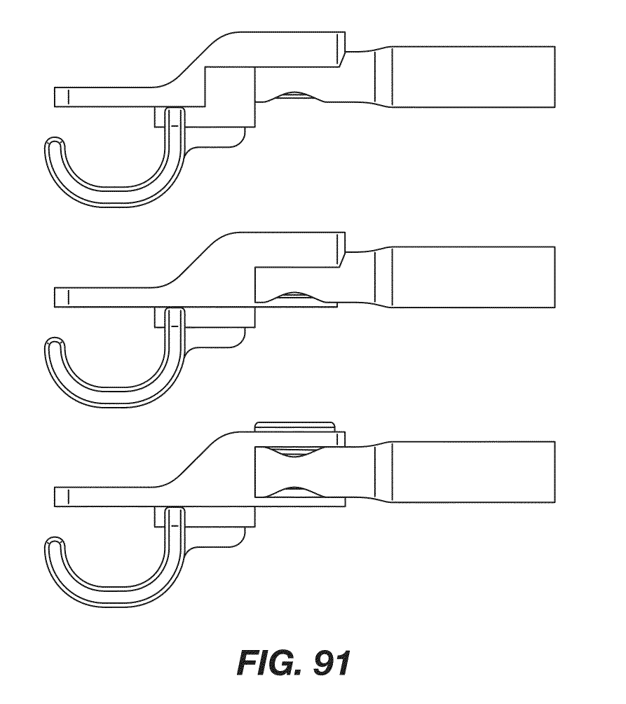

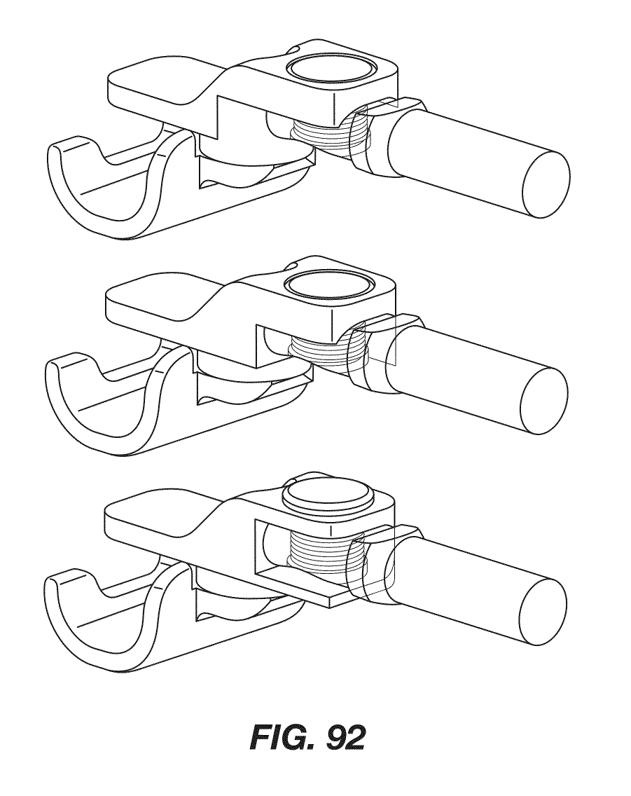

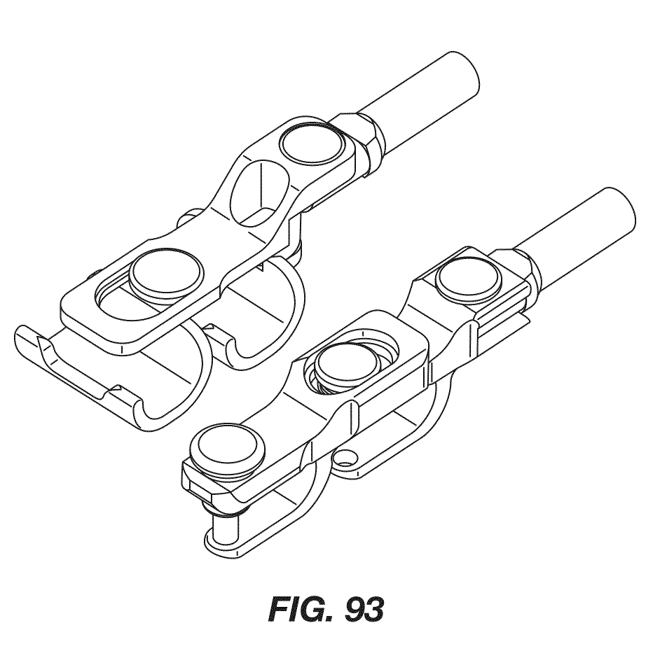

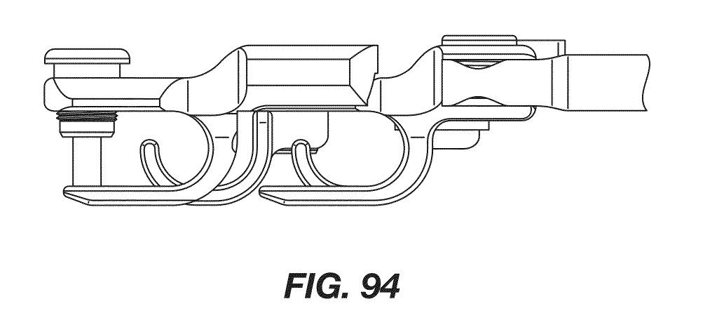

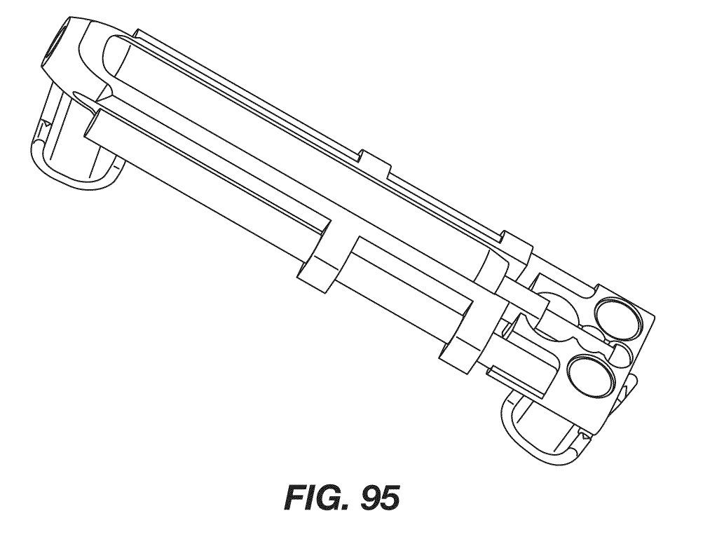

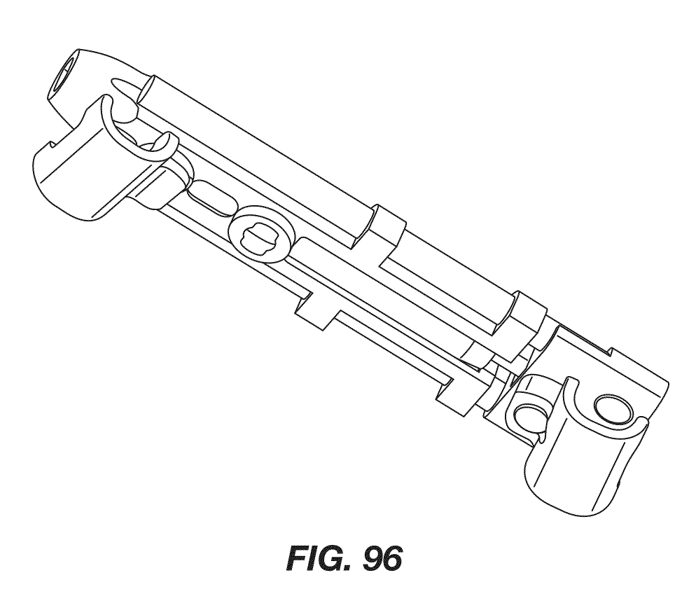

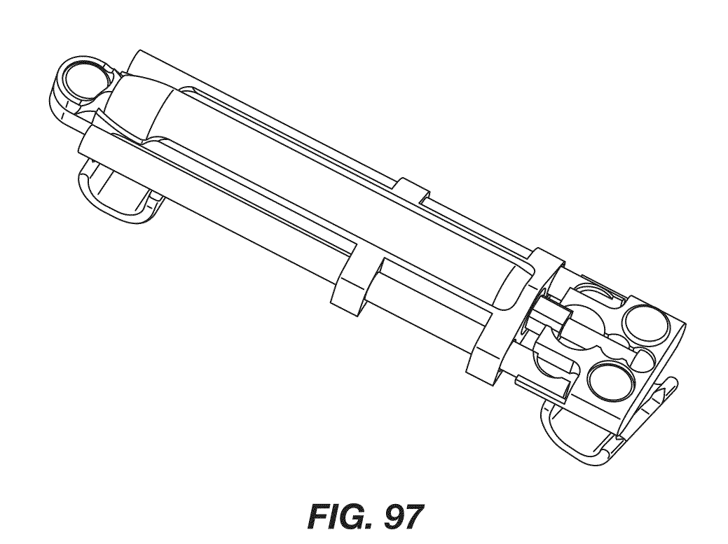

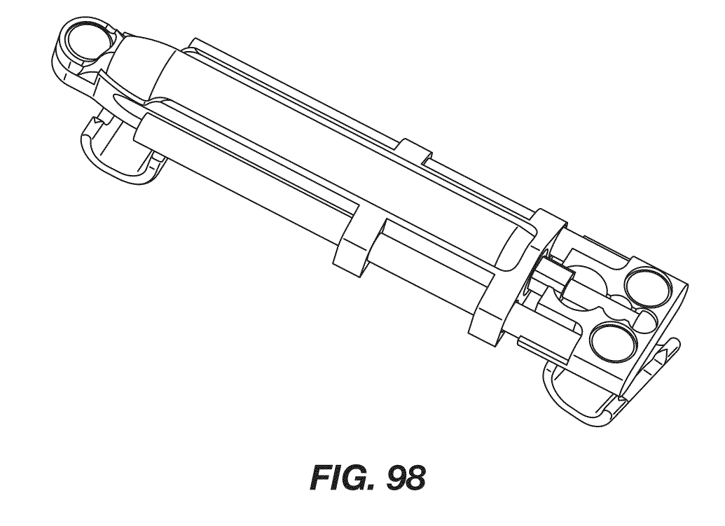

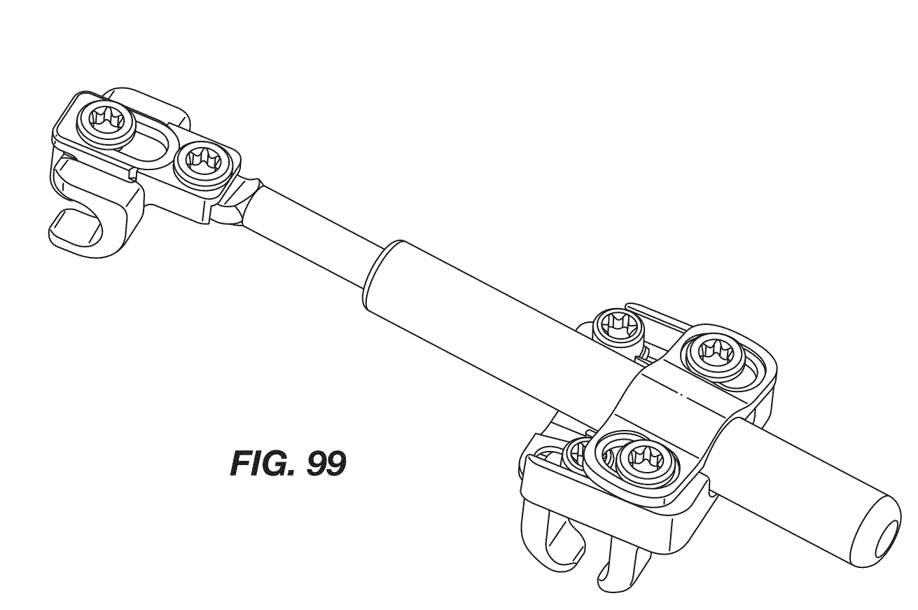

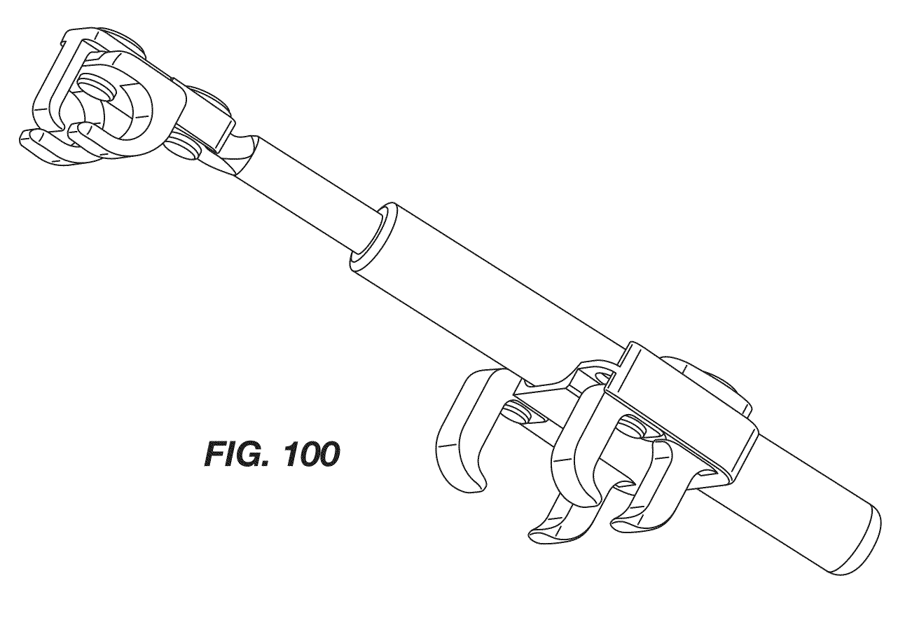

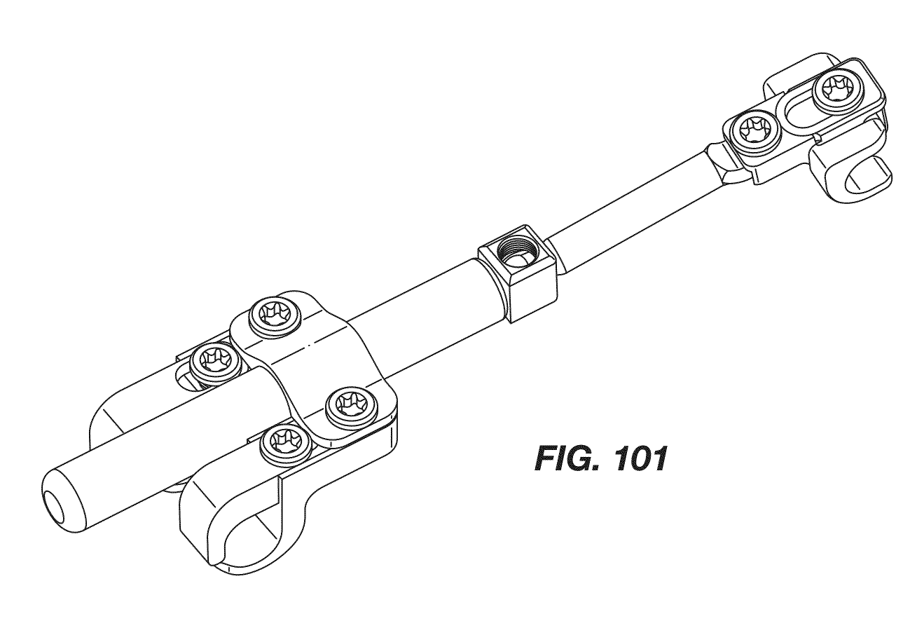

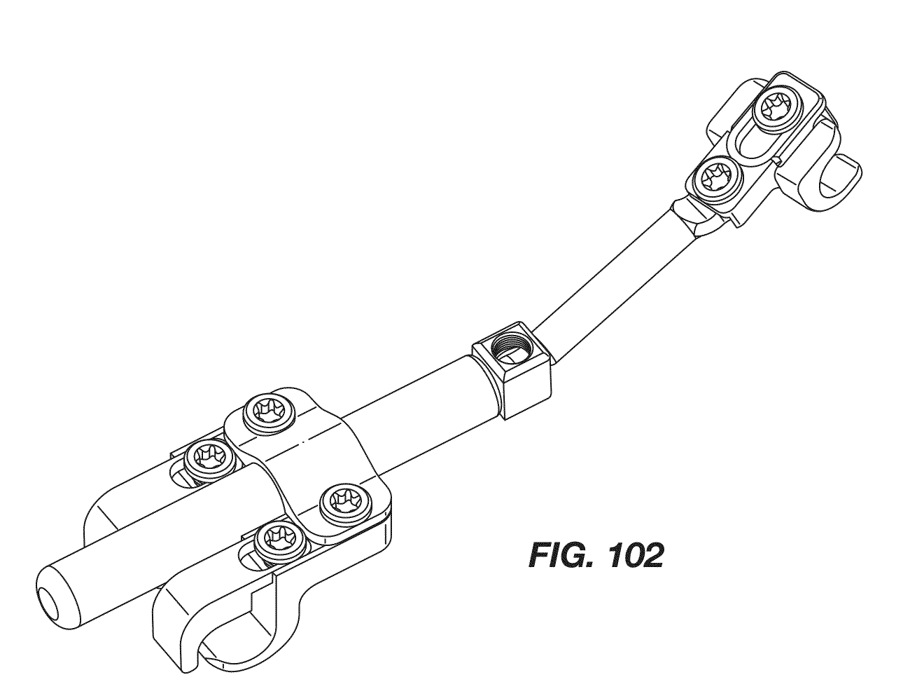

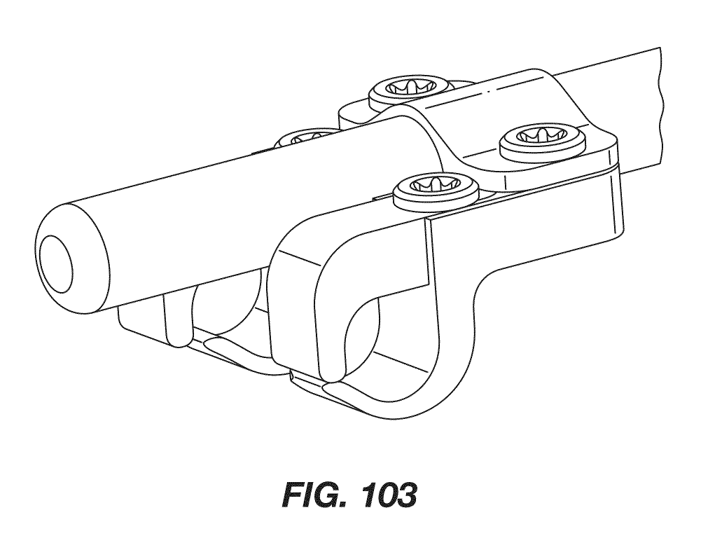

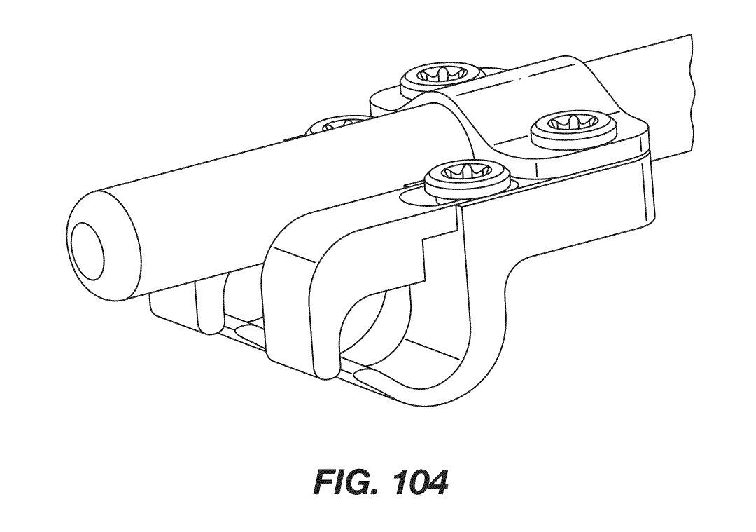

FIGS. 90-104 illustrate various other embodiments of surgical devices configured to facilitate spinal rod anchoring. FIG. 90 illustrates an embodiment of a narrow, fixed bone connector on the left and an embodiment of a wide, adaptable bone connector on the right. The bone connectors of FIG. 90 can each be configured to attach to a rib. FIGS. 91 and 92 illustrate three embodiments of wide, adaptable bone connectors. The bone connector on the bottom of FIGS. 91 and 92 is the same as the wide, adaptable bone connector of FIG. 90. FIGS. 93 and 94 illustrate two embodiments of bone connectors each including two rib seats each configured to seat one rib therein. The bone connectors of FIGS. 93 and 94 are thus each configured as tandem hooks. FIGS. 95-98 illustrate an embodiment of a bone connector having a separation of actuator and carrier to prevent moments acting on the actuator, as shown in FIG. 95. FIG. 96 illustrates that the bone connector is configured to adjust in longitudinal length and includes a body with multiple attachments via bayonet connection. FIGS. 97 and 98 illustrate that the bone connector includes an adaptable hook blade with screw fixation. FIGS. 99 and 100 illustrate an embodiment of a bone connector including a three-point connection connector (on the left side of FIGS. 99 and 100) configured to attach to a first bone and have three points of contact therewith, and including a four-point connection connector (on the right side of FIGS. 99 and 100) configured to attach to a second bone and have four points of contact therewith. FIGS. 101-104 illustrate another embodiment of a bone connector including a three-point connection connector (on the right side of FIGS. 101 and 102) configured to attach to a first bone and have three points of contact therewith, and including a four-point connection connector (in FIGS. 103 and 104 and on the left side of FIGS. 101 and 102) configured to attach to a second bone and have four points of contact therewith.

The devices discussed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before use. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam, and a liquid bath (e.g., cold soak).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A surgical device, comprising:
a bone connector that includes
a first connector having a first seat configured to seat a bone, and
a second connector having a second seat and a third seat each configured to seat the bone, the second connector being configured to move relative to the first connector when the first seat is seating the bone so as to cause the second and third seats to seat the bone with the first seat being positioned between the second and third seats such that the bone connector is configured to have three points of contact with the bone when the first and second connectors are connected to the bone;
wherein one of the first and second connectors is configured to seat an elongate rod such that the seated rod is movable relative to at least one of the first and second connectors in at least one direction.

2. The device of claim 1, wherein each of the first, second, and third seats includes an arcuate extension.

3. The device of claim 1, wherein the first seat includes an elongate post and each of the second and third seats includes an arcuate extension.

4. The device of claim 1, wherein the first and second connectors are configured to be secured in a fixed position relative to one another so as to secure the bone seated in the first, second, and third seats.

5. The device of claim 1, wherein the movement of the second connector relative to the first connector includes rotational movement of the second connector about a pivot point defined by the first connector.

6. The device of claim 1, wherein the movement of the second connector relative to the first connector includes longitudinal sliding movement of the second connector along a longitudinal axis defined by the first connector.

7. The device of claim 1, wherein the first and second connectors are movable along a longitudinal length of the seated rod.

8. The device of claim 1, wherein the seated rod is angularly movable relative to the first and second connectors.

9. The device of claim 1, wherein the first connector is configured to seat the elongate rod.

10. The device of claim 1, wherein the second connector is configured to seat the elongate rod.

11. The device of claim 1, further comprising a second bone connector that includes
a third connector having a fourth seat configured to seat a second bone, and
a fourth connector having a fifth seat and a sixth seat each configured to seat the second bone, the fourth connector being configured to move relative to the third connector when the fourth seat is seating the second bone so as to cause the fifth and sixth seats to seat the second bone with the fourth seat being positioned between the fifth and sixth seats such that the second bone connector is configured to have three points of contact with the second bone when the third and fourth connectors are connected to the second bone;
wherein one of the third and fourth connectors is configured to seat the elongate rod.

12. A surgical device, comprising:
a first connector including a first seat configured to seat a rib;
a second connector including a second seat configured to seat the rib, the first and second connectors being configured to be secured in a fixed position relative to one another with the rib seated by the first and second seats; and
an elongate rod configured to be seated by one of the first and second connectors and be movable relative thereto when the first and second connectors are in the fixed position relative to one another so as to allow adjustability of the elongate rod relative to the rib.

13. The device of claim 12, wherein the movement of the elongate rod relative to the first and second connectors includes sliding movement of the elongate rod along a longitudinal axis of the elongate rod.

14. The device of claim 12, wherein the movement of the elongate rod relative to the first and second connectors includes pivoting movement of the elongate rod about a pivot point defined by a connection point between one end of the elongate rod and the one of the first and second connectors that seats the elongate rod.

15. The device of claim 12, wherein the one of the first and second connectors is configured to have the elongate rod side loaded thereto.

16. The device of claim 12, wherein the one of the first and second connectors is configured to have the elongate rod end loaded thereto.

17. The device of claim 12, wherein the first connector includes a first opening therein, and the second connector includes a second opening therein; and
further comprising a set screw configured to be received in the first and second openings so as to fix the first and second connectors in the fixed position relative to one another with the rib seated by the first and second seats.

18. The device of claim 12, further comprising:
a third connector including a third curved seat configured to receive a second rib therein; and
a fourth connector including a fourth curved seat configured to receive the second rib therein;
wherein the elongate rod is configured to be seated by one of the third and fourth connectors and be movable relative thereto so as to allow adjustability of the elongate rod relative to the second rib.

19. The device of claim 12, wherein the first and second seats each include a curved seat.

20. The device of claim 12, wherein one of the first and second seats includes a curved seat and the other of the first and second seats includes an elongate post.

21-112. (canceled)
